(19) United States
(12) Patent Application Publication
(10) Pub. No.: US 2025/0331906 A1
(43) Pub. Date: Oct. 30, 2025

(54) DEVICES, SYSTEMS, AND METHODS FOR PULSED ELECTRIC FIELD TREATMENT OF TISSUE

(71) Applicant: Endogenex, Inc., Plymouth, MN (US)

(72) Inventors: Robert Anthony D'AGOSTINO, St. Louis Park, MN (US); Zackary John BECKER, Shoreview, MN (US); Matthew CAMBRONNE, Plymouth, MN (US); Michael Patrick BRENZEL, St. Paul, MN (US)

(21) Appl. No.: 19/187,226

(22) Filed: Apr. 23, 2025

Related U.S. Application Data
(60) Provisional application No. 63/638,851, filed on Apr. 25, 2024.
(60) Provisional application No. 63/707,018, filed on Oct. 14, 2024.

(51) Int. Cl. A61B 18/00; A61B 1/00; A61B 1/005; A61B 1/273

(52) U.S. Cl. CPC A61B 18/00; A61B 1/00135; A61B 1/005; A61B 1/273; A61B 2018/0016; A61B 2018/00184; A61B 2018/0022; A61B 2018/00321; A61B 2018/00339; A61B 2018/00404; A61B 2018/00488; A61B 2018/00494; A61B 2018/00875; A61B 2018/00904; A61B 2018/00982

(57) ABSTRACT

Described here are devices, systems, and methods for treating tissue. In some variations, a method of treating a target tissue may comprise advancing a tissue treatment system and a visualization device to the target tissue of a patient. The tissue treatment system may comprise a tissue treatment device comprising an elongate body, an expandable member including an electrode array, and a sheath. In a delivery configuration, the expandable member may be disposed in the sheath circumferentially about the visualization device in an unexpanded configuration. The expandable member may be advanced distal to the sheath. The expandable member may be transitioned into an expanded configuration. The target tissue may be treated using the tissue treatment device. The expandable member may be retracted to reposition the system to the delivery configuration.

### CROSS-REFERENCE TO RELATED APPLICATIONS

This application claims priority to U.S. Provisional Application No. 63/707,018, filed Oct. 14, 2024 and U.S. Provisional Application No. 63/638,851, filed Apr. 25, 2024, the entire contents of each of which are incorporated by reference in their entirety.

### TECHNICAL FIELD

Devices, systems, and methods herein relate to applying pulsed electric fields to tissue to treat a chronic disease, including but not limited to diabetes.

### BACKGROUND

Diabetes is a widespread condition, affecting millions worldwide. In the United States alone, over 20 million people are estimated to have the condition. Diabetes accounts for hundreds of billions of dollars annually in direct and indirect medical costs. Depending on the type (Type 1, Type 2, and the like), diabetes may be associated with one or more symptoms such as fatigue, blurred vision, and unexplained weight loss, and may further be associated with one or more complications such as hypoglycemia, hyperglycemia, ketoacidosis, neuropathy, and nephropathy.

The treatment of chronic diseases such as obesity and diabetes through duodenal resurfacing has been proposed. For example, removing the majority of the mucosal cells from the section of the large intestine nearest the stomach may allow a rejuvenated mucosal layer to be regenerated, thereby restoring healthy (non-diabetic) signaling. Conventional treatments that apply thermal energy to the duodenum risk excessively heating and thus damaging more layers of the duodenum (e.g., muscularis) than desired, and/or must compensate for this excessive thermal heating. Conversely, conventional solutions may generate incomplete and/or uneven treatment. As such, additional systems, devices, and methods for treatment of duodenal tissue may be desirable.

### SUMMARY

Described here are devices, systems, and methods for applying pulsed or modulated electric fields to tissue. These systems, devices, and methods may, for example, treat duodenal tissue of a patient to treat diabetes. In some variations, a system for treating tissue may comprise an elongate body, and an expandable member coupled to the elongate body. The expandable member may comprise an electrode array, a first portion, and a second portion. A sheath may at least partially receive a visualization device and the expandable member. The first portion may be positioned circumferentially about the visualization device in a first direction and the second portion may be positioned circumferentially about the visualization device in a second, opposite direction.

In some variations, the first lateral portion may be at least partially overlapped by the second lateral portion. In some variations, the sheath may be attached to the visualization device. In some variations, the elongate body may be configured to translate the expandable member relative to the sheath. In some variations, the elongate body may comprise one or more of an inflation lumen, a suction lumen, a pull wire, and a lead wire. In some variations, the electrode array may be coupled to the expandable member via a thermal seal. In some variations, the elongate body may comprise a suction lumen, the suction lumen at least partially disposed between the electrode array and expandable member such that suction is applied to the target tissue through the electrode array. In some variations, in an expanded configuration, the expandable member may comprise a first arc length and the electrode array may comprise a second arc length less than the first arc length. In some variations, the expandable member may comprise one or more of a thermoplastic urethane, thermoplastic elastomer, polyethylene terephthalate, polyimide, nylon, and biaxially-oriented polyethylene terephthalate. In some variations, the expandable member may be transparent.

In some variations, the system may further comprise a handle including an actuator configured to translate the elongate body relative to the sheath. In some variations, the system may further comprise a fastener configured to couple the elongate body to visualization device. In some variations, the expandable member may comprise a plurality of expandable members arranged serially. In some variations, the system may further comprise one more of a pressure sensor, temperature sensor, and proximity sensor. In some variations, the expandable member may further comprise a support member.

Also described herein is a system for treating tissue comprising an elongate body, an expandable member coupled to the elongate body, the expandable member comprising an electrode array and defining a longitudinal axis. The expandable member may be asymmetric relative to the longitudinal axis. A sheath may at least partially receive a visualization device and the expandable member. In a delivery configuration, the expandable member may be disposed between an inner surface of the sheath and an outer surface of the visualization device. In some variations, the expandable member may be disposed distal to the sheath in a treatment configuration.

In some variations, the electrode array may comprise a flexible circuit substrate, wherein the flexible circuit substrate comprises one or more of the group consisting of: all-Polyimide laminate, Polyester (PET), Polyethylene Naphthalate (PEN), Polyamide, Liquid Crystal Polymer (LCP), and PTFE.

In some variations, an actuator may be coupled to the elongate body. The actuator may be configured to transition the elongate body and expandable member between the delivery configuration and the treatment configuration by translating the elongate body and expandable member relative to the sheath.

In some variations, the expandable member may comprise a first asymmetric portion and a second asymmetric portion. When transitioning from the treatment configuration to the delivery configuration, the first asymmetric portion may be configured to be between the second asymmetric portion and the endoscope. In some variations, a combined diameter of the system in the delivery configuration may be less than about 17 mm. In some variations, the expandable member may further comprise a proximal tapered portion. In some variations, the proximal tapered portion and the longitudinal axis form an angle between about 10 degrees and about 80 degrees.

In some variations, the expandable member may be eccentrically coupled to the elongate body such that a longitudinal axis of the elongate body does not align with the longitudinal axis of the expandable member. In some variations, the elongate body may be coupled to a sidewall of the expandable member. In some variations, the proximal tapered portion may comprise a first lateral taper and a second lateral taper asymmetric to the first lateral taper.

Also described herein is a system for treating tissue comprising an elongate body, an inflatable balloon coupled to the elongate body, the inflatable balloon comprising an electrode array, a first lateral portion, and a second lateral portion. A sheath may at least partially house a visualization device and the inflatable balloon. When housed in the sheath, the first lateral portion may be rolled around the visualization device and the second lateral portion may be rolled around the visualization device and partially overlap the first lateral portion.

Also described herein is a system for treating tissue comprising an elongate body, an elongate body, an inflatable balloon coupled to the elongate body, the inflatable balloon comprising an electrode array and a pleat configured to facilitate flattening the inflatable balloon for placement into a delivery configuration. A sheath may at least partially house a visualization device and the inflatable balloon. In the delivery configuration, the inflatable balloon may at least be partially positioned circumferentially around the visualization device within the sheath.

In some variations, a distal portion of the inflatable balloon may comprise at least one pleat. In some variations, in an expanded configuration, the distal end of the inflatable balloon may comprise a rectangular shape. In some variations, a lateral portion of the inflatable balloon may comprise at least one pleat. In some variations, the pleat may be configured to stretch the target tissue. In some variations, the pleat may be configured to transition the inflatable balloon between a folded configuration having a first diameter and an unfolded configuration having a second diameter greater than the first diameter. In some variations at least part of the length of the inflatable balloon may comprise one or more pleats. In some variations, each one of two or more pleats may be radially spaced apart from adjacent pleats.

In some variations, the inflatable balloon may comprise a wall thickness of between about 0.02 mm and about 0.5 mm. In some variations, the inflatable balloon may comprise a seam formed via a thermal seal. In some variations, the inflatable balloon may comprise a length of between about 10 mm and about 300 mm. In some variations, the electrode array may be configured to generate a therapeutic electric field at a first tissue depth of about 1 mm and a non-therapeutic electric field at a second tissue depth of at least about 1.5 mm. In some variations, the electrode array may define one or more openings through the electrode array. In some variations, one or more of the elongate body and the visualization device may be configured to suction tissue through the one or more openings at a pressure between about 10 mmHg and about 200 mmHg.

In some variations, the electrode array may be configured to generate a therapeutic electric field that treats a predetermined set of cell types and not muscularis tissue. In some variations, the electrode array may be configured to generate a therapeutic electric field that treats cells but leaves intact tissue scaffolding. In some variations, the electrode array may comprise a plurality of elongate electrodes comprising a ratio of a center-to-center distance between proximate electrodes to a width of the electrodes between about 2.3:1 and about 3.3:1.

In some variations, the plurality of elongate electrodes may comprise a first electrode and a second electrode. The second electrode may be parallel to or interdigitated with the first electrode. In some variations, the center-to-center distance between proximate electrodes and the width of the plurality of elongate electrodes may be substantially equal. In some variations, at least one of the electrodes may comprise a semi-elliptical cross-sectional shape. In some variations, a ratio of a height of an electrode to a width of an electrode may be between about 1:4 and about 1:8.

In some variations, proximate electrodes may be spaced apart by a weighted average distance of between about 0.3 mm and about 6 mm. In some variations, electrodes of the electrode array may be spaced apart between about 0.5 mm and about 2 mm.

In some variations, a signal generator may be coupled to the electrode array. The signal generator may be configured to generate a pulse waveform comprising a frequency between about 250 kHz and about 950 kHz, a pulse width between about 0.5 μs and about 4 μs, a voltage applied by the electrode array of between about 100 V and about 2 kV, and a current density between about 0.6 A and about 100 A from the electrode array per square centimeter of tissue.

Also described herein is a method of treating tissue comprising advancing a pulsed electric field device to a target tissue of a patient. The pulsed electric field device may comprise advancing a tissue treatment system and a visualization device to a first target tissue of a patient. The tissue treatment system may comprise a tissue treatment device comprising an elongate body, an expandable member including an electrode array, and a sheath. In a delivery configuration, the expandable member may be disposed in the sheath circumferentially about the visualization device in an unexpanded configuration. The expandable member may be advanced distal to the sheath. The expandable member may be transitioned into an expanded configuration. The target tissue may be treated using the tissue treatment device. The expandable member may be retracted to reposition the system into the delivery configuration.

In some variations, the expandable member may be visualized using the visualization device positioned within the sheath. In some variations, the treatment device may be repositioned and treat a second target tissue. In some variations, the treatment device may be repositioned proximally to treat the second target tissue before retracting the expandable member to reposition the system to the delivery configuration. In some variations, the first lateral portion may at least partially overlap with the second lateral portion in the delivery configuration.

In some variations, overlapping the first lateral portion at least partially with the second lateral portion in the delivery configuration may comprise positioning the first portion circumferentially about the visualization device in a first direction and positioning the second portion circumferentially about the visualization device in a second, opposite direction.

In some variations, advancing the expandable member distal to the sheath may comprise translating the elongate member relative to the sheath. In some variations, transitioning the expanded member to an expanded configuration may comprise inflating the expandable member via an inflation lumen of the elongate body. In some variations, suction may be applied to a portion of the target tissue through the expandable member. In some variations, the target tissue of the patient may be sized by advancing a sizing device. In some variations, sizing the target tissue of the patient may be based on a pressure measurement.

In some variations, treating the target tissue may treat a metabolic disorder comprising one or more of obesity, Non-alcoholic fatty liver disease (NAFLD), Nonalcoholic steatohepatitis (NASH), proinflammatory processes, immunological processes, Alzheimer's disease, neurological disorders, Type I diabetes, and Type II diabetes. In some variations, the target tissue may comprise one or more of a duodenum, a pylorus, an esophagus, a stomach, a small intestine, a large intestine, a vasculature, a thoracic cavity, an abdomino-pelvic cavity, a pelvic cavity, a vertebral cavity, and a cranial cavity.

Also described herein is a system for treating tissue comprising a visualization device comprising a handle and a distal portion, and an inflation lumen coupled to the handle of the visualization device. A inflatable balloon may be coupled to a distal end of the inflation lumen. The inflatable balloon may comprise an electrode array, a distal pleat, and a proximal portion decreasing in diameter toward a proximal end of the inflatable balloon. A sheath may comprise a lumen coupled to the proximal portion of the visualization device, the sheath at least partially receiving the inflation lumen and the inflatable balloon within the lumen. In a delivery configuration, the inflatable balloon may be positioned within the sheath lumen and a first portion of the inflatable balloon may be rolled around the visualization device in a first direction and a second portion of the inflatable balloon may be rolled around the visualization device in a second, opposite direction. In some variations, more than one inflatable balloon may be provided, wherein adjacent inflatable balloons may be longitudinally spaced-apart from each other and/or provided in a serial configuration. In some variations, the adjacent inflatable balloons may touch while in other variations the adjacent inflatable balloons are in a non-touching configuration.

Also described herein is a tissue treatment device comprising an overtube or sheath including a lumen and a window positioned along a sidewall of the overtube, the lumen and the window each configured to receive a visualization device therethrough. A n expandable member may be coupled to the overtube or sheath. The expandable member may be configured to treat tissue.

In some variations, the window may be adjacent and proximal to the expandable member. In some variations, the window may comprises a width of at least an outer diameter of the visualization device and a length greater than the width.

In some variations, a distal end of the overtube may be coupled to an inner diameter of the expandable member. In some variations, the overtube may comprise a support disposed opposite the window, the support configured to increase a stiffness of the overtube. In some variations, the support may comprise one or more of coil reinforcement and braid reinforcement. In some variations, the overtube may comprise a stiffness of about 0.1 times to about 10 times a stiffness of a visualization device having a diameter configured to be disposed within the tissue treatment device. In some variations, the overtube may comprise one or more of an inflation lumen, a suction lumen, a pull wire, and a lead wire.

In some variations, the expandable member may be configured to generate a therapeutic electric field. In some variations, the expandable member may comprise a treatment member configured to treat the tissue. In some variations, the treatment member may comprise one or more of an electrode, an electrode array, a piezoelectric transducer, a laser, a blade, and a thermal element. In some variations, the treatment member may comprise an electrode array coupled to the expandable member via a thermal seal.

In some variations, the expandable member may comprise a balloon. In some variations, the balloon may comprise at least one pleat configured to transition the balloon between a folded configuration having a first diameter and an unfolded configuration having a second diameter greater than the first diameter. In some variations, a lateral portion of the expandable member may comprise at least one pleat. In some variations, the expandable member may comprise one or more of a thermoplastic urethane, thermoplastic elastomer, polyethylene terephthalate, polyimide, nylon, and biaxially-oriented polyethylene terephthalate.

In some variations, a system comprising the treatment device may further comprise the visualization device slidably positioned within the lumen of the overtube. In some variations, the visualization device may be configured to advance through the window to visualize the expandable member.

Also described herein is a method of treating a target tissue comprising advancing a tissue treatment system and a visualization device to the target tissue of a patient where the tissue treatment system may comprises a tissue treatment device comprising an overtube defining a lumen and a window, and an expandable member coupled to the overtube. The visualization device may be advanced through the window of the overtube. The expandable member may transition into an expanded configuration. The target tissue may be treated using the expandable member.

In some variations, the method may include the step of advancing the visualization device through the lumen of the overtube and distal to the expandable member before advancing the tissue treatment system and the visualization device to the target tissue. In some variations, the method may include the step of advancing the tissue treatment device relative to the visualization device such that the window is distal to the visualization device after advancing the tissue treatment system and the visualization device to the target tissue.

In some variations, the method may include the step of visualizing one or more of the expandable member and the target tissue after the visualization device is advanced through the window of the overtube. In some variations, the method may include the step of visualizing the target tissue comprises identifying an ampulla of the duodenum. In some variations, the method may include the step of applying suction to a portion of the target tissue through one or more of the visualization device and the lumen of the overtube.

In some variations, the method may include the step of re-treating the target tissue one or more times using the expandable member. In some variations, the method may include the step of visualizing the treated target tissue before re-treating the target tissue.

In some variations, the method may include the step of repositioning the tissue treatment device after treating a first target tissue, and treating a second target tissue. In some variations, the method may include the step of visualizing the treated target tissue before treating the second target tissue.

In some variations, the treatment device may be repositioned proximally or distally of the first target tissue to treat the second target tissue after transitioning the expandable member into the delivery configuration. In some variations, the treatment device may be rotatably repositioned to treat the second target tissue after transitioning the expandable member into the delivery configuration.

In some variations, transitioning the expandable member to the expanded configuration may comprise inflating the expandable member via an inflation lumen of the overtube or sheath. In some variations, the tissue treatment system and the visualization device may be advanced to the target tissue of a patient in a delivery configuration where the expandable member is in an unexpanded configuration. In some variations, the method may include the step of transitioning the expandable member back into the delivery configuration.

In some variations, treating the target tissue may treat a metabolic disorder comprising one or more of obesity, Non-alcoholic fatty liver disease (NAFLD), Nonalcoholic steatohepatitis (NASH), proinflammatory processes, immunological processes, Alzheimer's disease, neurological disorders, Type I diabetes, and Type II diabetes. In some variations, treating the target tissue may treat Barrett's esophagus. In some variations, the target tissue may comprise one or more of a duodenum, a pylorus, an esophagus, a stomach, a small intestine, a large intestine, a vasculature, a thoracic cavity, an abdomino-pelvic cavity, a pelvic cavity, a vertebral cavity, and a cranial cavity.

A Iso described herein is a treatment device comprising an elongate body and a balloon coupled to the elongate body. The balloon may comprise at least one pleat configured to facilitate flattening of the expandable member for placement into a delivery configuration. A width of the pleat in the delivery configuration is about 0.1 mm to about half of a difference between a width of the expandable member in the delivery configuration and a diameter of the elongate body. The balloon may be configured to treat tissue.

In some variations, at least one pleat may comprise a first pleat on a first side of the balloon and a second pleat on a second side of the expandable member opposite the first side of the balloon. In some variations, the treatment device may comprise an electrode array configured to treat the tissue. The electrode array may be spaced apart from one or more of a proximal end and a distal end of the expandable member by at least about 0.25 inches. In some variations, an electrode array may be coupled to the balloon. In some variations, at least one pleat may be configured to transition the balloon between a folded configuration having a first diameter and an unfolded configuration having a second diameter greater than the first diameter. In some variations, a lateral portion of the balloon may comprise at least one pleat. In some variations, the balloon may comprise one or more of a thermoplastic urethane, thermoplastic elastomer, polyethylene terephthalate, polyimide, nylon, and biaxially-oriented polyethylene terephthalate.

A Iso described herein is a treatment device comprising an elongate body and an expandable member coupled to the elongate body. The expandable member may comprise an electrode array and a plurality of pleats configured to facilitate flattening of the expandable member in an unexpanded configuration. When transitioning the expandable member to an expanded configuration, a portion comprising the electrode array may expand before the plurality of pleats unfold.

In some variations, each pleat of the plurality of pleats may comprise a first tapered portion coupled to a second tapered portion. In some variations, each of the plurality of pleats may fold inwards in the unexpanded configuration. In some variations, the portion of the expandable member comprising the electrode array may comprise a first rigidity and one or more other portions of the expandable member comprise a second rigidity different than the first rigidity. In some variations, a width of each pleat of the plurality of pleats may be up to half of a difference between a width of the electrode array and a diameter of the elongate body.

In some variations, the electrode array may comprise a plurality of electrodes. The device may be configured to maintain a predetermined spacing between the electrodes of the plurality of electrodes when the expandable member is expanded from an unexpanded configuration to a diameter of between about 15 mm and about 45 mm.

In some variations, the expandable member may comprise a balloon. In some variations, the plurality of pleats may be configured to transition the balloon between a folded configuration having a first diameter and an unfolded configuration having a second diameter greater than the first diameter. In some variations, a lateral portion of the expandable member may comprise at least one pleat of the plurality of pleats. In some variations, the expandable member may comprise one or more of a thermoplastic urethane, thermoplastic elastomer, polyethylene terephthalate, polyimide, nylon, and biaxially-oriented polyethylene terephthalate.

Also described herein is a method of manufacturing a tissue treatment device comprising the steps of disposing an electrode array on a surface of an expandable member where the electrode array may comprise a substrate comprising one or more apertures along a perimeter of the substrate. A bonding layer may be disposed over the electrode array. The electrode array may be bonded between the expandable member and the bonding layer using the apertures of the substrate.

In some variations, the method may include the step of attaching a first longitudinal edge of the expandable member to a second longitudinal edge of the expandable member to define a lumen of the expandable member. In some variations, the first end of the expandable member may comprise a first portion of a pleat, and the second end of the expandable member may comprise a second portion of the pleat.

In some variations, the method may include the step of forming at least one pleat in the expandable member. In some variations, the method may include the step of coupling an elongate body to an inner surface of the expandable member.

In some variations, the bonding layer covers a perimeter of the electrode array. In some variations, the electrode array may be bonded to the expandable member and the bonding layer using one or more of heat, pressure, an adhesive, and a chemical. In some variations, the expandable member may comprise one or more of a thermoplastic urethane, thermoplastic elastomer, polyethylene terephthalate, polyimide, nylon, and biaxially-oriented polyethylene terephthalate.

A Iso described herein is a treatment device comprising an elongate body, an expandable member coupled to the elongate body, and an electrode array coupled to the expandable member where the electrode array may comprise a substrate defining a plurality of apertures. A bonding layer may be bonded to the electrode array and the expandable member using the apertures of the substrate.

In some variations, the bonding layer covers a perimeter of the electrode array. In some variations, the substrate may be sandwiched between the bonding layer and the expandable member. In some variations, the elongate body may be an overtube or sheath. In some variations, the expandable member may comprise a balloon. In some variations, the bonding layer may be bonded to the expandable member through the electrode array using one or more of heat, pressure, an adhesive, and a chemical. In some variations, the expandable member may comprise one or more of a thermoplastic urethane, thermoplastic elastomer, polyethylene terephthalate, polyimide, nylon, and biaxially-oriented polyethylene terephthalate.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a cross-sectional representation of a gastrointestinal tract showing various anatomical structures.

FIG. 1B is a cross-sectional representation of a duodenum.

FIG. 2A is a cross-sectional schematic view of a portion of the small intestine.

FIG. 2B is a cross-sectional schematic view of a portion of the small intestine.

FIG. 2C is a cross-sectional schematic view of a portion of the small intestine.

FIG. 3A is a cross-sectional image of a duodenum.

FIG. 3B illustrates a cross-sectional view of duodenal tissue.

FIG. 3C illustrates a cross-sectional view of duodenal tissue.

FIG. 3D illustrates a cross-sectional view of duodenal tissue.

FIG. 3E illustrates a cross-sectional view of duodenal tissue.

FIG. 3F illustrates a cross-sectional view of duodenal tissue.

FIG. 4 is a block diagram of an illustrative variation of a tissue treatment system such as a pulsed electric field system.

FIG. 5A is a perspective view of an illustrative variation of a tissue treatment system in a delivery configuration.

FIG. 5B is a perspective view of an illustrative variation of a tissue treatment system in a treatment configuration.

FIG. 6A illustrates a side view of an illustrative variation of a tissue treatment system in a treatment configuration.

FIG. 6B illustrates a side view of an illustrative variation of a tissue treatment system in a treatment configuration.

FIG. 6C illustrates a side view of an illustrative variation of a tissue treatment system in a delivery configuration.

FIG. 7A illustrates a side cutaway view of an illustrative variation of a tissue treatment system in a treatment configuration.

FIG. 7B illustrates a side cutaway view of an illustrative variation of a tissue treatment system in a treatment configuration.

FIG. 7C illustrates a perspective cutaway view of an illustrative variation of a tissue treatment system in a treatment configuration.

FIG. 7D illustrates a side cutaway view of an illustrative variation of a tissue treatment system in a treatment configuration. illustrates a side cutaway view of an illustrative variation of a tissue treatment system in a treatment configuration.

FIG. 7E illustrates a side cutaway view of an illustrative variation of a tissue treatment system in a treatment configuration.

FIG. 7F illustrates a side cutaway view of an illustrative variation of a tissue treatment system in a treatment configuration.

FIG. 7G illustrates a side cutaway view of an illustrative variation of a tissue treatment system in a treatment configuration.

FIG. 7H illustrates a perspective cutaway view of an illustrative variation of a tissue treatment system in a treatment configuration.

FIG. 7I illustrates an end view of an illustrative variation of a tissue treatment system in a treatment configuration.

FIG. 7J illustrates a side cutaway view of an illustrative variation of a tissue treatment system in a treatment configuration.

FIG. 7K illustrates a side cutaway view of an illustrative variation of a tissue treatment system in a treatment configuration.

FIG. 7L illustrates a side cutaway view of an illustrative variation of a tissue treatment system in a treatment configuration.

FIG. 7M illustrates a side cutaway view of an illustrative variation of a tissue treatment system in a treatment configuration.

FIG. 7N illustrates a side cutaway view of an illustrative variation of a tissue treatment system in a treatment configuration.

FIG. 7O illustrates a side cutaway view of an illustrative variation of a tissue treatment system in a treatment configuration.

FIG. 7P illustrates a side cutaway view of an illustrative variation of a tissue treatment system in a treatment configuration.

FIG. 7Q illustrates a side cutaway view of an illustrative variation of a tissue treatment system in a treatment configuration.

FIG. 7R illustrates a side cutaway view of an illustrative variation of a tissue treatment system in a treatment configuration.

FIG. 7S illustrates a side cutaway view of an illustrative variation of a tissue treatment system in a treatment configuration.

FIG. 8A illustrates a side view of an illustrative variation of an expandable member.

FIG. 8B illustrates a side view of an illustrative variation of an expandable member.

FIG. 8C illustrates a side view of an illustrative variation of an expandable member.

FIG. 8D illustrates a side view of illustrative variations of an expandable member.

FIG. 9A illustrates a side view of illustrative variation of an expandable member in an illustrative configuration.

FIG. 9B illustrates a side view of illustrative variation of an expandable member in an illustrative configuration.

FIG. 9C illustrates a side view of illustrative variation of an expandable member in an illustrative configuration.

FIG. 9D illustrates a side view of illustrative variation of an expandable member in an illustrative configuration.

FIG. 10A illustrates a side view of an illustrative variation of an expandable member in an illustrative expanded configuration.

FIG. 10B illustrates an end view of an illustrative variation of an expandable member in an illustrative expanded configuration.

FIG. 10C illustrates a perspective view of an illustrative variation of an expandable member in an illustrative expanded configuration.

FIG. 10D illustrates a side view of an illustrative variation of an expandable member in an illustrative expanded configuration.

FIG. 11A illustrates a side view of an illustrative variation of an expandable member in an illustrative expanded configuration.

FIG. 11B illustrates a side view of an illustrative variation of an expandable member in an illustrative expanded configuration.

FIG. 11C illustrates a side view of an illustrative variation of an expandable member in an illustrative expanded configuration.

FIG. 11D illustrates a side view of an illustrative variation of an expandable member in an illustrative expanded configuration.

FIG. 11E illustrates a side view of an illustrative variation of an expandable member in an illustrative unexpanded configuration.

FIG. 11F illustrates a side view of an illustrative variation of an expandable member in an illustrative unexpanded configuration.

FIG. 12A illustrates an end perspective view of an illustrative variation of an expandable member transitioning between an illustrative unexpanded configuration and an illustrative expanded configuration.

FIG. 12B illustrates an end perspective view of an illustrative variation of an expandable member transitioning between an illustrative unexpanded configuration and an illustrative expanded configuration.

FIG. 12C illustrates an end perspective view of an illustrative variation of an expandable member transitioning between an illustrative unexpanded configuration and an illustrative expanded configuration.

FIG. 12D illustrates an end perspective view of an illustrative variation of an expandable member transitioning between an illustrative unexpanded configuration and an illustrative expanded configuration.

FIG. 13A illustrates a side view of an illustrative variation of an expandable member transitioning between an unexpanded configuration and an expanded configuration.

FIG. 13B illustrates a side view of an illustrative variation of an expandable member transitioning between an unexpanded configuration and an expanded configuration.

FIG. 13C illustrates a side view of an illustrative variation of an expandable member transitioning between an unexpanded configuration and an expanded configuration.

FIG. 13D illustrates a side view of an illustrative variation of an expandable member transitioning between an unexpanded configuration and an expanded configuration.

FIG. 13E illustrates a side view of an illustrative variation of an expandable member transitioning between an unexpanded configuration and an expanded configuration.

FIG. 13F illustrates a side view of an illustrative variation of an expandable member transitioning between an unexpanded configuration and an expanded configuration.

FIG. 14 illustrates a block diagram of an illustrative variation of a signal generator.

FIG. 15 illustrates a flowchart describing an illustrative variation of a method of treating tissue.

FIG. 16 illustrates a flowchart describing another illustrative variation of a method of treating tissue.

FIG. 17A illustrates a schematic diagram of an illustrative variation of a pulse waveform for treating tissue.

FIG. 17B illustrates a schematic diagram of an illustrative variation of a pulse waveform for treating tissue.

FIG. 18A illustrates a schematic diagram of illustrative variations of a method of treating tissue.

FIG. 18B illustrates a schematic diagram of illustrative variations of a method of treating tissue.

FIG. 18C illustrates a schematic diagram of illustrative variations of a method of treating tissue.

FIG. 19A illustrates a schematic diagram of illustrative variations of a method of treating tissue.

FIG. 19B illustrates a schematic diagram of illustrative variations of a method of treating tissue.

FIG. 19C illustrates a schematic diagram of illustrative variations of a method of treating tissue.

FIG. 19D illustrates a schematic diagram of illustrative variations of a method of treating tissue.

FIG. 19E illustrates a schematic diagram of illustrative variations of a method of treating tissue.

FIG. 19F illustrates a schematic diagram of illustrative variations of a method of treating tissue.

FIG. 20A illustrates a side cutaway view of an illustrative variation of the present disclosure.

FIG. 20B illustrates a side cutaway view of an illustrative variation of the present disclosure.

FIG. 20C illustrates a side cutaway view of an illustrative variation of the present disclosure.

FIG. 20D illustrates a side cutaway view of an illustrative variation of the present disclosure.

FIG. 20E illustrates a side cutaway view of an illustrative variation of the present disclosure.

FIG. 21A illustrates an end view of an illustrative variation of the present disclosure.

FIG. 21B illustrates a side cutaway view of an illustrative variation of the present disclosure.

FIG. 21C illustrates a side view of a deflated illustrative variation of the present disclosure.

FIG. 21D illustrates a side view of the illustrative variation of FIG. 21C in an inflated configuration.

FIG. 22A illustrates a top view of an illustrative variation of the present disclosure.

FIG. 22B illustrates a top cutaway view of an illustrative variation of the present disclosure.

FIG. 23A illustrates a top cutaway view of an illustrative variation of the present disclosure.

FIG. 23B illustrates a side cutaway view of an illustrative variation of the present disclosure.

FIG. 24A illustrates a side cutaway view of an illustrative variation of the present disclosure.

FIG. 24B illustrates a side cutaway view of an illustrative variation of the present disclosure.

FIG. 25 illustrates an illustrative variation of a method of the present disclosure.

FIG. 26 illustrates an illustrative variation of a method of the present disclosure.

### DETAILED DESCRIPTION

Described herein are devices, systems, and methods for treating tissue to address a chronic disease. For example, a pulsed electric field (PEF) system may be configured to generate a therapeutic pulsed electric field having predetermined bipolar, high current, short duration, electric pulses and applied to any body cavity or lumen (e.g., organ, vasculature, vessel) of a patient. The PEF treatment described herein may increase cell permeability and induce a targeted, non-thermal cellular necrosis while preserving Extracellular Matrix (ECM) tissue scaffold, promoting rapid epithelial layer replacement that reestablishes a neuroendocrine cell population with minimal inflammation. In this manner, a depth of penetration may be controlled and smooth muscle cells may be preserved, thereby leaving surrounding tissue undamaged.

In some variations, devices, systems, and methods may include those for treating diabetes by treating tissue within the gastrointestinal tract (e.g., duodenal tissue) of a patient. In some variations, treatment of the duodenum may comprise treating at least about 30% of the mucosal lining of the duodenum with minimal trauma, damage or scarring to the submucosa, vasculature, and muscles. For example, a mucosal and submucosal cells of the duodenum may be treated using a pulsed electric field (PEF) system configured to generate a therapeutic pulsed electric field. The application of a pulsed electric field to duodenal tissue may affect individual parts or mechanisms within a cell (e.g., depth of tissue treated), that can be specifically targeted based on electrode geometry and the frequency, intensity, and duration of the pulses.

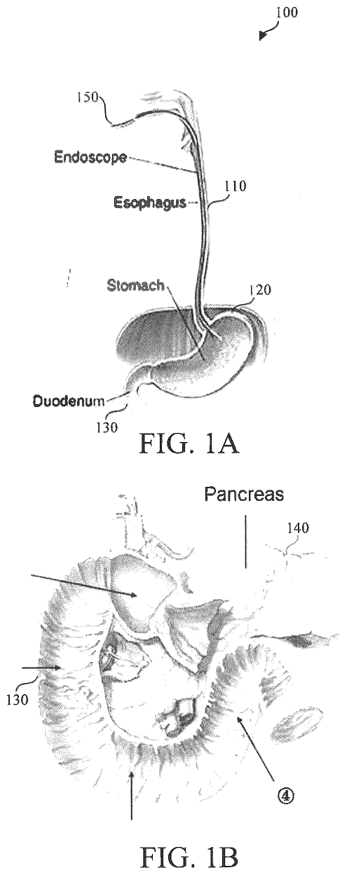

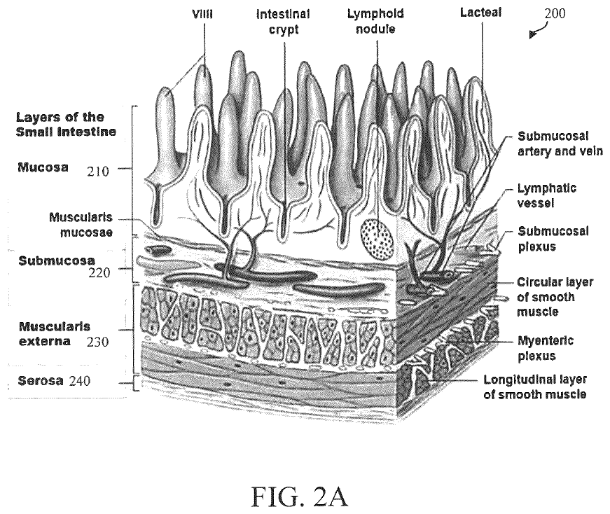

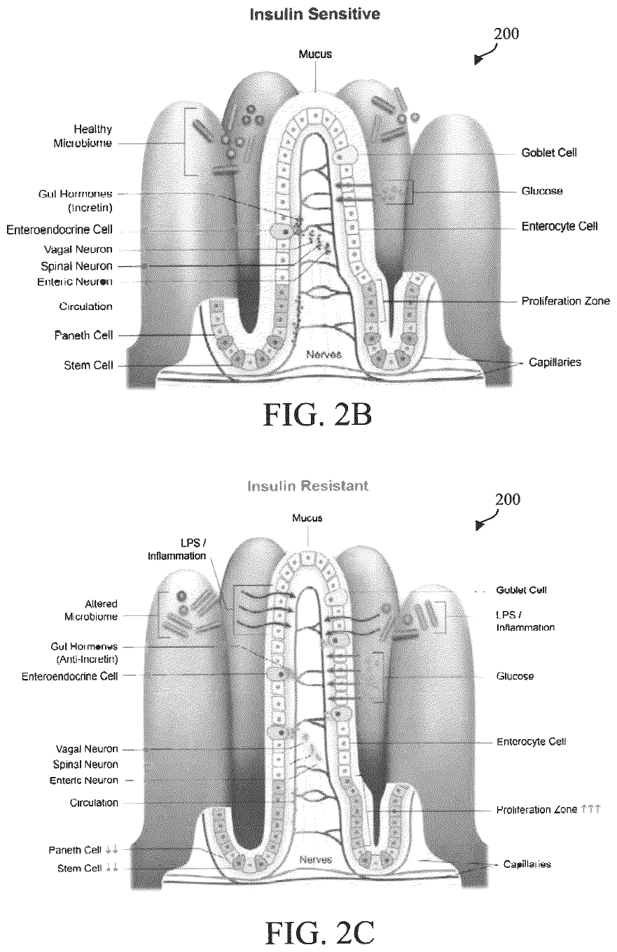

It may be helpful to briefly identify and describe the relevant small intestine anatomy. FIG. 1A is a cross-sectional view of the gastrointestinal tract of a patient (100). Shown there is a visualization device (150) (e.g., endoscope) advanced into the stomach (120) through the esophagus (110). The stomach (120) is connected to the duodenum (130). FIG. 1B is a detailed cross-sectional view of the duodenum (130), which surrounds the head of the pancreas (140). The duodenum is a “C” shaped hollow jointed tube structure that is typically between about 20 cm and about 35 cm in length and between about 20 mm and about 45 mm in diameter. FIGS. 2A-2C are cross-sectional schematic views of the layers of the small intestine (200) including the mucosa (210), submucosa (220), muscularis externa (230), and serosa (240). Treatment of the duodenum may comprise resurfacing the mucosa (210) as described herein. Access to the gastrointestinal tract (e.g., duodenum, stomach, large intestine) may be performed by advancing the systems and devices described herein through one or more of the esophagus, stomach, pylorus, lower esophageal junction, crackle pharyngeal junction, and several acute small radius bends throughout the length of the digestive tract.

It may further be helpful to briefly discuss electroporation and the role of ohmic heating. Electroporation is the application of an electric field to living cells to cause ions of opposite charge to accumulate on opposite sides of cell membranes. Generally, electroporation requires a potential difference across the cell membrane on the order of about 0.5 to about 1 volt and for a cumulative duration on the order of about 1 to about 2 milliseconds. Electroporation necessarily generates ohmic heating but there is considerable confusion in the literature about this, including a significant number of references that incorrectly assert the existence of non-thermal electroporation. For example, an external uniform electric field of magnitude E applied to an intracellular fluid with ionic conductivity σ_{ic} will generate a current density Eσ_{ic} and dissipate a thermal power density E²σ_{ic}. If the medium has a heat capacity Cₚ and density ρ, the resulting rate of temperature rise is given by equation (1):

$$\frac{dT}{dt} = \frac{{E}^{2} {σ}_{ic}}{{C}_{p} ρ} \qquad eqn . ( 1 )$$

For example, a 1 KV/cm electric field acting on tissue with a conductivity of about 0.3 S/m, a heat capacity of about 3.7 joule/(gm° C.), and a density of about 1 gm/cc will heat the tissue at a rate of about 800° C./second. Note that, without current passing through the tissue, there is no electric field in the tissue since the tissue is an ionic conductor. The initial time after an external field is abruptly applied to the membrane to accumulate charge may be on the order of about 30 nanoseconds, which suggests that, during an initial membrane-charging phase, the average temperature rise may be in the tens of microdegrees. When an external electric field is applied, and ionic currents have charged the membrane surfaces to collapse the field into the lipid bilayers, leakage current may still flow, though the heating may be confined to the membranes for sub-microsecond timescales. For example, using a lipid layer conductivity of σₗᵢ=0.002 S/m, a 1 volt potential across an 8 nm layer may locally heat at an instantaneous rate of about 8° C./microsecond. This heating rate drops with time from the application of the external electric field, as the heat may diffuse further from the membrane.

If the ionic currents are confined to pores in the cell membranes, current crowding will cause the heating rate in the pores to be correspondingly higher. Since the pore area might be 1% or less of the membrane area, the current density in the pores may be one hundred times higher than in the bulk tissue. This gives a ten thousand times increase in heating rate, leading to local heating rates on the order of 10° C./microsecond.

Local temperature rise is a contributing mechanism to the transition from electroporation to irreversible electroporation. Thermal diffusion lowers the local temperature excursions. For example, assuming a tissue thermal diffusivity κ of 0.13 mm²/s, the thermal diffusion length at 10 μsec is \sqrt{(10 μs)(0.13 mm²/s)} or 1.1 micron, which is much larger than a typical pore. At 1 millisecond, the thermal diffusion length is on the order of the cell size, so the localized heating effects may be ignored.

The bulk tissue remains a good ionic conductor during the electroporation treatment, heating at a rate on an order of magnitude of about 800° C./s while the external field is being applied. If the external field is removed, the cell membranes may discharge on the order of about 30 nanoseconds, obliging the continued application of external voltage and current to induce pore formation and growth. As the maximum tolerable temperature rise of the bulk tissue may be on the order of about 13° C., the maximum duration that the external field may be applied, even in a bipolar configuration, may be within an order of magnitude of about 10 milliseconds. As this heat is generated to a treatment depth in the tissue of about several millimeters, the required time to cool the tissue by conduction may be about 70 seconds (e.g., (3 mm²)/(0.13 mm²/sec)). Blood convection likely dominates the observed cooling times that are on the order of about 10 seconds. Electroporation may also increase with the temperature of the bulk tissue due to the phase transition of the lipid cell membrane, which for some cells on the duodenum is 41° C. The phase transition temperature may be the temperature required to induce a change in the lipid physical state from the ordered gel phase to the liquid crystalline phase.

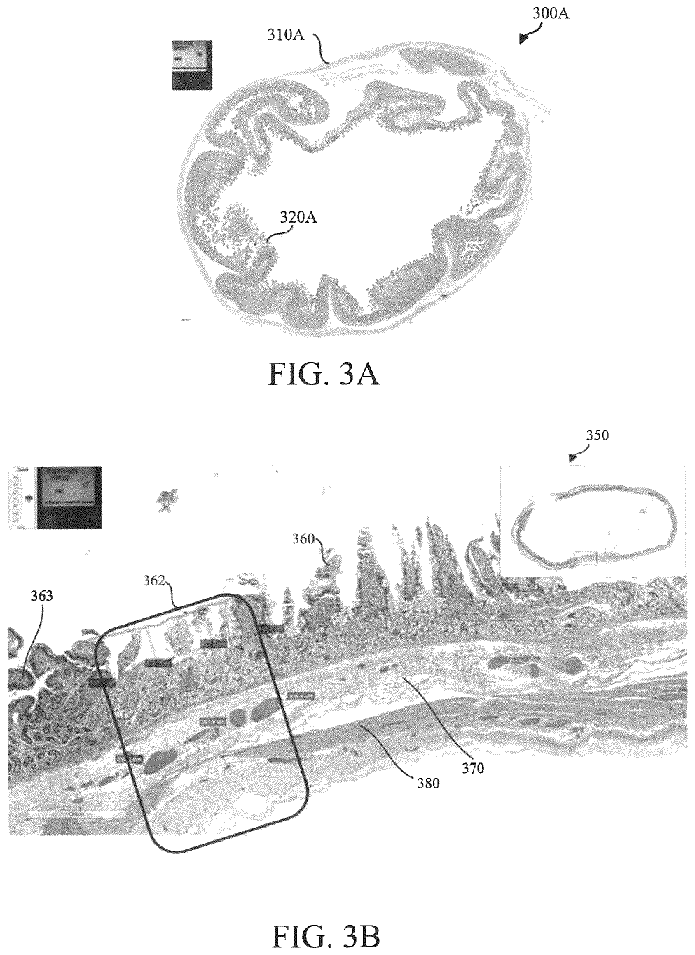

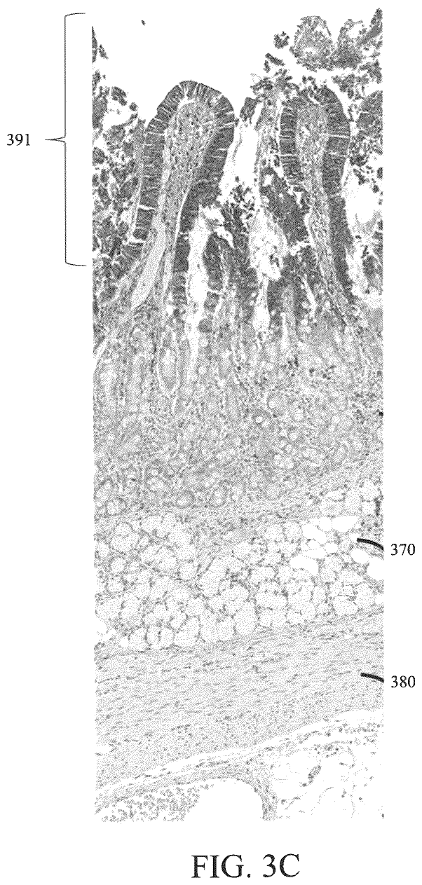

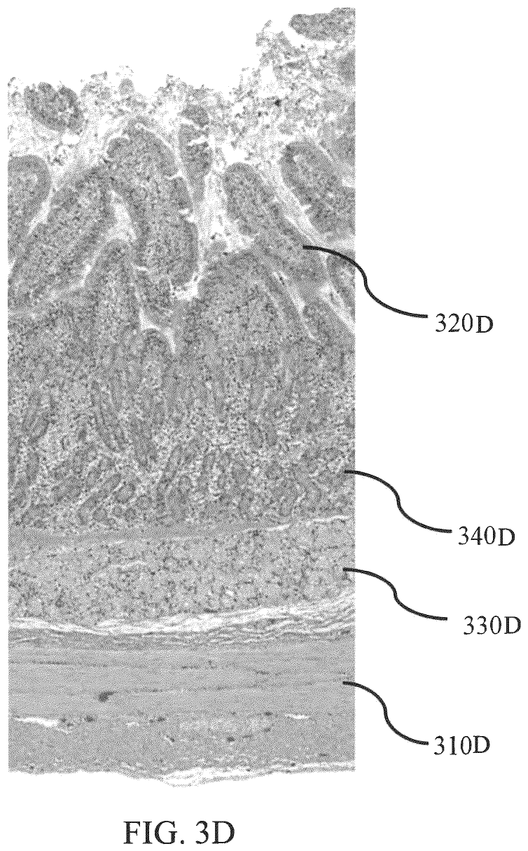

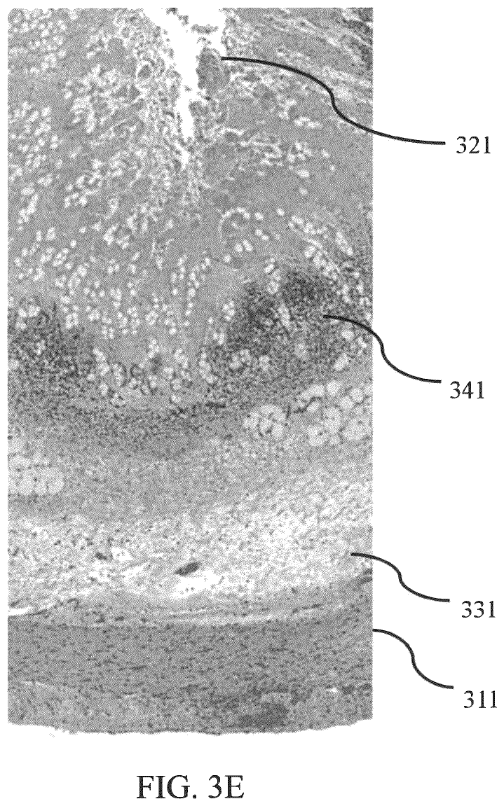

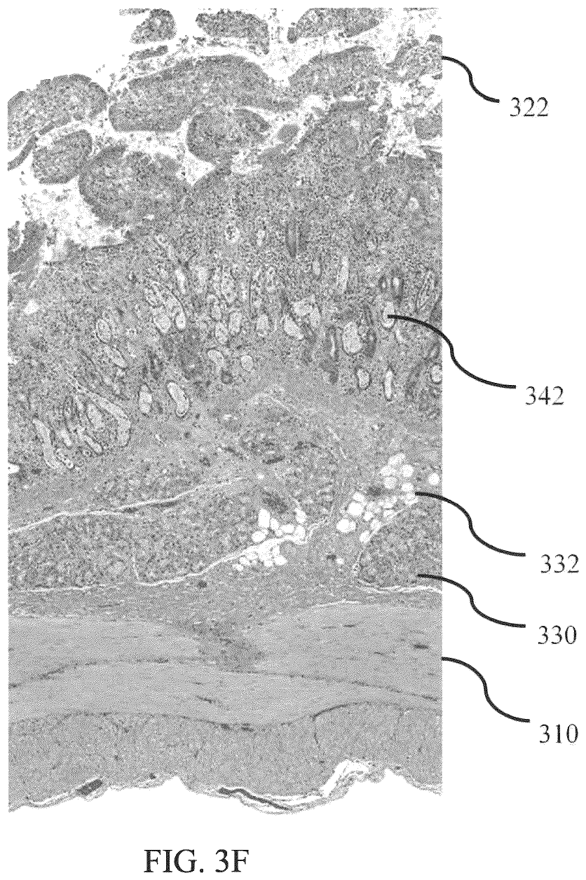

Electroporation parameters may be varied to produce different effects on tissue. FIG. 3A is a cross-sectional image of an untreated duodenum (300A) including a muscular layer (310A) and villi (320A). FIG. 3D is an image of an illustrative variation of duodenal tissue in its native untreated state including a muscularis layer (310D), submucosa (330D), villus crypts (340D) and villi (320D). As described in more detail herein, FIG. 3E depicts duodenal tissue that has undergone majority thermal heat treatment and FIG. 3F depicts duodenal tissue that has undergone majority pulsed or modulated electric field treatment. The treatments described herein (e.g., FIG. 3F), which primarily treat the mucosa layer with preserved tissue architecture appearing similar to the native tissue, reduces trauma to tissue relative to the thermal treatment shown in FIG. 3E.

The application of a pulsed electric field to tissue results in non-thermal tissue changes. For example, FIG. 3D is an image of normal untreated (e.g., native tissue) porcine duodenal mucosa. FIG. 3F is an image of the initial mucosal histologic appearance with evolving epithelial loss and lamina propria structural/architectural preservation. For example, FIG. 3F depicts the histologic evolution with complete native epithelial loss and early crypt regeneration within the preserved lamina propria. The glandular layer across FIGS. 3A-3D and 3F demonstrates the structural preservation of the lamina propria following treatment. For example, histopathology confirms that the PEF treatment as described herein applied at a depth of about 1 mm in duodenal tissue will treat the mucosal layer without the pulsed electric field energy affecting the muscularous propria at a therapeutic level.

In some variations, a pulsed electric field (PEF) treatment may be combined with localized thermal treatment. For example, thermal treatment may be applied to surface tissue or near-surface tissue while PEF treatment may be applied to relatively deeper tissue. As described in more detail herein, the depth of tissue treatment received by one or more layers may be adjusted based on one or more of electrode design, applied voltage, time or duration of energy delivery, frequency of applied energy, and tissue configuration. An example of such control is thermal treatment applied up to a tissue depth of about 0.1 mm and a PEF treatment applied to a tissue depth of up to about 1 mm. The ratio and depth of thermal treatment to PEF treatment may be based on a desired clinical outcome (e.g., effect). In some variations, thermal treatment may be applied up to a tissue depth of about 3 mm, and PEF treatment may be applied up to a tissue depth of about 5 mm. Therefore, in some variations, more thermal treatment than PEF treatment may be applied to tissue. Based on a depth or type of tissue, different healing cascades maybe optimal. In some variations, the villas mucosa at up to about 1 mm may be thermally treated to allow substantially the entire tissue architecture to be replaced, while the submucosa may be PEF treated to preserve the tissue architecture and promote rapid healing of that layer. Furthermore, neither the thermal treatment nor PEF treatment may affect the deeper muscularis propria layer.

FIG. 3B is an image of an illustrative variation of duodenal tissue that has undergone different treatments. In particular, the tissue (360) was treated with pulsed or modulated electric field energy and first mucosa region (362) was further subjected to radiofrequency (RF) ablation energy. The ablated villi of the first mucosa region (362) have broken cellular membranes and destroyed cell structures such that those cells are no longer viable or functioning. By contrast, a second mucosa region (360) has cells that have undergone cell lysis where the cellular membranes remain intact but the cells are no longer viable and functioning. That is, cell lysis corresponds to functional cell death with intact cellular structures while ablation refers to loss of both cell structure and function. The submucosa (370) and muscularis (380) remain healthy (e.g., viable and fully functioning with cell integrity). In FIG. 3B, villi in the first mucosa region (362) are thermally ablated while the cell lysis in the second mucosa region (360) is generated by a pulsed or modulated electric field. A third mucosa region (363) adjacent to the thermal lesion of the first mucosa region (362) is not treated at all and comprises viable tissue.

FIG. 3C illustrates a histological slide of the duodenum from tissue about 24 hours after treatment with heat and pulsed electric field, showing a partial treatment of the mucosa down to the crypt layer, with injured cells. A fourth mucosa region (391) corresponds to thermal/heat fixed tissue of the villi, including the villi-associated enteroendocrine cells. The fourth mucosa region (391) demonstrates architectural and cytological preservation with cellular detail with hyperchromatic nuclear and hypereosinophilic cytoplasmic staining. Overall, interstitial hemorrhage and infiltrating post-treatment-associated inflammatory cells are not identified. The heat fixed tissue may be expected to slough off, followed by surface re-epithelialization and villous structural healing with crypt cell repopulation. The crypt tissues are partially affected by a combination of heat and pulsed electric field effects. The tissue healing timeline is expected to be longer than that of a pulsed electric field treatment without thermal effect. The submucosa (370) and muscularis (380) are histologically unaffected. FIG. 3E is an image of an illustrative variation of 24 hour porcine duodenal histology following an isolated hyperthermic tissue treatment (i.e., no concomitant pulsed electrical field exposure) which destroys the lamina propria in that tissue scaffolding is burned and destroyed, and will be sloughed off and removed during healing. This demonstrates the histologic features of a thermal tissue dose, consistent with thermal/heat-induced coagulative necrosis without thermal/heat fixation. In this region, the glandular epithelium and neuroendocrine cells (321) show a loss of cytologic detail, consistent with cellular “ghost images.” Interstitial hemorrhage and reactive inflammatory cells of the mucosal layer (341) are present at the region's edge. The submucosa (331) and muscularis (311) also show injury related changes. This region may be anticipated to heal similar to an ischemic-type coagulative necrosis with resorption and remodeling with mucosal regeneration. The thermal lesion destroyed the lamina propria. Scaffolding is burned and destroyed and will be sloughed off and removed during healing. The tissue healing time frame for this region should be longer than that expected for a pulsed electric field treatment.

FIG. 3F is an image of an illustrative variation of duodenal tissue that has undergone treatment with pulsed or modulated electric field energy to a controlled depth not including the muscularis, untreated muscularis propria layer (310), submucosa (330), treated submucosa (332), treated villus crypts, with partial cell lysis and maintained tissue scaffolding (342), and treated villi with villas sloughing (322). The treated submucosa (332) also maintains tissue scaffolding. These treated tissues illustrate cells that have undergone a cell death where the cellular membranes remain intact but the cells are no longer viable and functioning. The healing cascade will replace these cells without infiltration of large number of inflammatory cells, and the surface will re-epithelialize and with villous structural healing and crypt cell repopulation. The muscularis (310) remains healthy (e.g., viable and fully functioning with cell integrity) without therapeutic effect from the pulsed electric field energy. That is, with pulsed or modulated electric field energy cell death corresponds to functional cell death with intact cellular structures while ablation refers to loss of both cell structure and function and an aggressive necrotic inflammatory response healing cascade.

In some variations, a target depth of treatment includes the mucosal layer but excludes treatment of the muscularous propria. Human tissue data assessed through histopathology supports about a 1 mm target depth for PEF tissue treatment where the pulsed electric field does not penetrate through to the muscularous propria at a therapeutic level. Based on the methods described herein, the healing response may be essentially completed in about thirty days. Moreover, the systems, devices, and methods described herein may provide uniform treatment coverage throughout a circumference and length of the duodenum.

Some methods for treating diabetes may include treating the submucosa layer of the duodenum without treating the muscularis. Conventional solutions do not consistently treat the submucosa layer without negatively impacting the muscularis. Instead, conventional solutions may add complicated mitigating steps such as lifts with saline injection in an attempt to protect the muscularis. For reference, the mucosal layer typically has a thickness between about 0.5 mm to about 1 mm, the submucosa layer typically has a thickness of about 0.5 mm and about 1 mm, and the muscularis typically has a thickness of about 0.5 mm. Inducing injury to the muscularis may result in adverse clinical outcomes. Furthermore, the anatomical structure along a circumference of the duodenum is not uniform, thus complicating efforts to treat just the submucosa and not the muscularis.

The methods described herein may selectively change tissue viability without losing the integrity of the majority of the treated tissue by applying a predetermined pulsed or modulated electric field and, optionally, without other treatment of the tissue to mitigate the pulsed or modulated electric field to a portion of tissue. By contrast, RF based energy treatment may predominantly generate heat-induced cell lysis (e.g., cell death) or ablation that may indiscriminately damage tissue and destroy cellular structure, and which may be difficult to modulate, thus negatively impacting treatment outcomes. In some variations, the methods described here may comprise applying a pulsed or modulated electric field to thermally-induce local necrotic cell death (e.g., local ablation) for tissue immediately adjacent to an electrode array and to induce cell lysis (e.g., functional cell death) within a predetermined range of tissue depths of (e.g., up to about 1 mm, between about 0.5 mm and 0.9 mm) while minimizing the physiological impact to tissue greater than the selected depth.

FIG. 3F is an image of an illustrative variation of duodenal tissue that has undergone treatment with pulsed or modulated electric field energy to a controlled depth. In FIG. 3F, the muscularis layer (310) and a portion of the submucosa (330) are untreated (i.e., energy delivered to tissue does not affect the tissue) and the villus crypts (342), villi (322) and a different portion of the submucosa (332) have been treated. Thus, the treatment applied to the duodenal tissue shown in FIG. 3F results in a more superficial (e.g., closer to the tissue surface) treated submucosa (332) and a deeper, untreated muscularis layer (310). The treated tissues contain cells that have undergone cell lysis where the tissue scaffolding remain intact but the cells are no longer viable and functioning. A mild healing cascade will replace these cells. The muscularis (310) adjacent to the treated submucosa (332) remains healthy (e.g., viable and fully functioning with cell integrity).

The pulsed or modulated electric fields near an electrode array may generate some thermal heating of tissue leading to tissue ablation that destroys both cell structure and function. However, cell lysis in tissue resulting from the pulsed or modulated electric fields applied herein are at least 50% pore-induced and less than 50% heat-induced such that a majority of cell death comprises functional cell death with intact cellular structures. For example, the thermal heating generated by a pulsed or modulated electric field is generally localized to a relatively small radius from each electrode of an electrode array and does not affect deeper layers of tissue such as the muscularis.

The systems, devices, and methods described herein may deliver energy to provide treatment characteristics optimized for each tissue layer to improve treatment outcomes. Near the surface of the tissue (e.g., less than about 0.5 mm, between about 0.1 mm and about 0.5 mm), thermal heating may generate local necrotic cell death of tissue that may slough off after treatment. At a tissue depth of between about 0.5 mm and about 1.3 mm (e.g., mucosa of duodenum), cell lysis may be generated by the pulsed or modulated electric field while thermal heating is limited (e.g., to less than about a 13° C. increase or 6° C. increase). For example, an electric field strength at about 1.0 mm may be about 2.5 kV/cm. At tissue depths beyond 1.0 mm, the energy delivered to tissue generates reversible electroporation with even less thermal heating such that deeper tissue may be substantially untreated. Thus, thermal heating may be limited to a surface tissue layer (e.g., less than about 0.5 mm, between about 0.1 mm and about 0.5 mm) while still delivering pulsed or modulated electric field energy for cell lysis of the mucosa.

For example, FIG. 3C is an image of an illustrative variation of duodenal tissue that has undergone a method of treating duodenal tissue described herein where villi (391) has been treated by a combination of thermal heating (e.g., more than 50%) and pore-induced cell death (e.g., less than 50%). The pulsed or modulated electric field applied to the villus crypts and submucosa (370) has treated the tissue to a majority (e.g., more than 50%) of pore-induced cell death with a lesser contribution (e.g., less than 50%) of cell death due to thermal heating. The muscularis (380) is substantially untreated by the pulsed or modulated electric field or other methods. For example, the submucosa in FIG. 3C is not subject to saline injection. The depth of treatment may be controlled such that a predetermined portion of the mucosal layer such as the villus crypts may remain untreated if desired. The configuration and geometry of the electrode arrays as described herein may enable the tissue treatment characteristics described herein.

By contrast, conventional solutions that apply other forms of thermal energy (e.g., steam, radiofrequency, laser, heated liquid) to the duodenum thermally ablate through multiple layers of the tissue (e.g., inducing more than 50% heat-induced necrotic cell death and less than 50% pore-induced cell death), thereby destroying the cellular structure of the mucosa at similar depths and which may detrimentally thermally damage the muscularis. In an attempt to mitigate the risk of unintentional thermal damage during application of thermal energy to deeper layers (e.g., muscularis) of the duodenum, saline may be injected into portions of duodenal tissue (e.g., the submucosa (330)). This additional step further complicates the procedure and is not always sufficient to prevent unwanted thermal tissue damage. The pulsed or modulated electric field based methods described here eliminate this additional step and provide greater protection against unwanted tissue damage by improving the energy delivery characteristics generated by a pulsed electric field device.

In some variations, pulsed electric field treatment may be applied while monitoring and/or minimizing tissue temperature increases. For example, a predetermined rise in tissue temperature (e.g., about 1° C., about 2° C., about 3° C.) may be followed by a pause (e.g., of a predetermined time interval) in energy delivery to allow the tissue to cool. In this manner, the total energy delivered may increase the tissue temperature below a predetermined threshold (e.g., below a safety limit). In some variations, the predetermined threshold may be up to about 3° C., about 6° C., about 10° C., about 13° C., including all ranges and sub-values in-between. In further variations, the predetermined threshold may be between about 1° C. to about 20° C., such as about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., or about 20° C. In some variations, the pulsed electric field treatment applied to tissue increases a tissue temperature by no more than about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., or about 20° C.

Moreover, the difficulty faced by conventional solutions in controlling unwanted thermal tissue damage would lead one of ordinary skill away from using the pulsed or modulated electric field energy levels and methods described herein. In some variations, the tissue power densities generated by a pulsed or modulated electric field may be several orders of magnitude higher than the tissue power densities generated by radiofrequency ablation. For example, a power density ratio of an analogous design for radio frequency ablation may be about 576 where a radiofrequency device is driven at about 25 Vᵣₘₛ and a pulsed electric field device is driven at about 600 Vᵣₘₛ. Thus, it would be unexpected for the pulsed or modulated electric field methods described here to not only treat tissue, but to do so without excess thermal tissue damage requiring mitigation procedures. Furthermore, the increased power densities may require additional insulation and protection of the pulsed electric field device, as well as a signal generator capable of generating such peak power levels. Generally, a duty cycle for PEF treatment may be several orders of magnitude lower than radio frequency ablation in order to keep a bulk tissue temperature rise below the predetermined threshold. For example, radio frequency ablation energy may generally be delivered continuously for several seconds. Accordingly, the duty cycle for PEF treatment may be between about 0.0000001 to about 0.001, about 0.000001 to about 0.001, about 0.00001 to about 0.001, about 0.00002 to about 0.001, about 0.00003 to about 0.001, about 0.00003 to about 0.0005, about 0.00003 to about 0.0004, or about 0.000035 to about 0.0004, including about 0.0000001, about 0.000001, about 0.00001, and about 0.00002. For example, in some variations, PEF treatment may collectively accumulate about 5 milliseconds of ON time over about 10 seconds, for a net duty cycle of about 0.0005.

Some conventional tissue treatment systems used in a patient body utilize a separate endoscope for visualization where each of the tissue treatment device (e.g., elongate body and expandable member) and endoscope are independently advanced during a procedure. Conventional systems and methods of treating tissue require precise coordination between the tissue treatment device and endoscope that may make procedures more challenging. For example, an esophagus may allow translation of a system having a diameter of about 17 mm such that a diameter (e.g., about 13 mm) of a conventional endoscope allows only about 4 mm of margin. Therefore, many conventional tissue treatment devices that utilize expandable members such as, for example, inflatable balloons, are advanced through an esophagus independently of an endoscope, and typically utilize fluoroscopic imaging to ensure proper advancement and positioning of the tissue treatment device and endoscope within the patient body. Reliance on fluoroscopic imaging may increase the procedure time and complexity of a procedure.

Moreover, some conventional tissue treatment systems are configured to transition between different configurations (e.g., size, shape, geometries) and may inadvertently couple (e.g., pinch, trap) tissue to one or more portions of the tissue treatment system, thereby damaging tissue, hindering a procedure, generating incomplete and/or uneven treatment, and/or requiring additional manipulation and repositioning of the tissue treatment system and/or endoscope. For example, a tissue treatment system may have a first configuration (e.g., smaller size) when initially advanced into the patient and a second configuration (e.g., larger size) when located at a tissue treatment site. However, transitioning between different configurations may conventionally alter the electrode geometry (e.g., electrode spacing) such that the electric field applied to tissue may be different between the first and second configurations. These differences in electrode geometry may have significant impacts on field strength, field depth, heat generation, and the like. Furthermore, conventional methods of manufacturing conventional tissue treatment systems do not minimize changes in electrode geometry due to configuration changes.

The systems and devices described herein may provide for reduced procedural and/or device complexity while reducing the need for additional imaging (e.g., fluoroscopic imaging). For example, a tissue treatment device (e.g., pulsed electric field device) may be coupled to a visualization device (e.g., endoscope), thereby facilitating coordination of the procedure such that additional imaging (e.g., fluoroscopic imaging) is unnecessary for determining the position of the tissue treatment device relative to the visualization device. In some variations, the visualization device may be configured to be disposed (e.g., slidable) within a lumen of the tissue treatment device to provide imaging for the visualization device and the tissue treatment device as they are advanced together (e.g., 1:1 translation) through tissue, thus reducing or eliminating the need for fluoroscopic imaging. For example, the tissue treatment device may comprise an overtube configured to receive the visualization device. The visualization device may be configured to translate through the elongate body (e.g., overtube) such that a distal end of the visualization device may be advanced distal to a distal end of the tissue treatment device such that the tissue treatment device “rides over” the visualization device. Additionally, the visualization device may be configured to advance through a window (e.g., aperture, slot, opening, hole) defined within a distal portion (e.g., sidewall) of the elongate body to provide visualization of an expandable member and/or target tissue. When a distal end of the visualization device is advanced through the window and disposed external to the tissue treatment device, as well as proximal to an expandable member of the tissue treatment device, the visualization device may be positioned to provide visualization (and optionally additional functionality such as, for example, suction, fluid delivery, tool access, and like) of the expandable member and/or target tissue. Accordingly, the visualization device may allow for visualization of tissue and/or a desired anatomical or other landmark (e.g., ampulla, proximal edge of treatment site, distal edge of treatment region, fiducial marking, etc.) both distal to the tissue treatment device and proximal to an expandable member of the tissue treatment device by translating the visualization device through the elongate body (e.g., overtube). Furthermore, a same operator handling the tissue treatment device may also handle the visualization device such that an additional operator is not needed.

In some variations, the tissue treatment device may be configured to transition between different configurations, thereby limiting a size of the treatment system when in a delivery configuration, allowing for simpler delivery. In addition, the structure of the expandable member from any of the delivery configuration, treatment configuration, and configuration in-between reduce tissue trapping and entanglement compared to conventional systems, and may also maintain electrode geometry to provide consistent energy delivery across different configurations (e.g., diameters). For example, an expandable member of a tissue treatment device may comprise an electrode array and a plurality of pleats configured to facilitate flattening of the expandable member in an unexpanded configuration. The pleats may be configured such that when they unfold, the expandable member increases in diameter while a predetermined electrode spacing of an electrode array is maintained, thereby ensuring that a shape and strength of a pulsed electric field generated by the electrode array maintains predetermined characteristics. Furthermore, the pleats may have a different rigidity than a portion of the expandable member having the electrodes in order to promote the transition of the expandable member to an expanded configuration while maintaining the electrode geometry.

In some variations, a tissue treatment device of the present invention may be disposed circumferentially about a visualization device within a sheath (e.g., delivery catheter) such that a distal portion of the tissue treatment device is disposed between an inner surface of the sheath and an outer surface of the visualization device. The tissue treatment device may, for example, include an expandable member and an electrode array configured to transition from a delivery configuration to a treatment configuration, configured to generate a pulsed electric field for treating tissue. For example, without limiting the patient anatomy to be treated, in variations in which the devices and systems described herein are utilized to treat the duodenum, the energy delivered by the tissue treatment device may regenerate mucosal and submucosal cells of the duodenum. The expandable member may be configured to transition between the delivery configuration and the treatment configuration without capturing or damaging tissue.

Generally, the devices described herein may comprise an elongate body and an expandable member coupled to the elongate body. The expandable member may comprise an electrode array. A sheath may at least partially receive a visualization device and the expandable member. In some variation, the sheath may also at least partially receive the elongate body. The expandable member may be positioned circumferentially about (e.g., rolled, folded, wrapped, disposed around) the visualization device such that the expandable member may be disposed between an inner surface of the sheath and an outer surface of the visualization device. In some variations, portions of the expandable member may overlap. Additionally or alternatively, the expandable member may be asymmetric relative to the longitudinal axis of the expandable member. In some variations, the expandable member may further comprise one or more pleats configured to facilitate appropriate expandable member sizing and tissue apposition, in addition to flattening the expandable member for placement into the delivery configuration.

Also described herein are methods for treating a target tissue. In some variations, a method of treating tissue may comprise advancing a tissue treatment system to a target tissue. The tissue treatment system may comprise a tissue treatment device and a visualization device, and the visualization device may be advanced to the target tissue of a patient when the visualization device while disposed within a lumen of the tissue treatment device. The tissue treatment device may be advanced to the target tissue in a compressed or unexpanded configuration where an expandable member of the tissue treatment device is in an unexpanded configuration. At the target tissue, the visualization device may be advanced through a window of an elongate body (e.g., overtube) of the tissue treatment device for visualization of the expandable member and/or the target tissue. The expandable member may transition to an expanded configuration and suction may optionally be applied to the target tissue using a lumen of the visualization device, a lumen of the elongate body (e.g., via the lumen of the elongate body housing the visualization device, via a separate lumen of the elongate body) or a separate suction device. The target tissue may be treated using the tissue treatment device. In some variations, the same target tissue may undergo a plurality (two, three, four, five or more) of treatments (e.g., re-treatment). Additionally or alternatively, the tissue treatment device may be repositioned (e.g., translationally, rotationally) to treat another portion of the target tissue. The target tissue may include one or more of a duodenum, a pylorus, an esophagus, a stomach, a small intestine, a large intestine, a vasculature, a thoracic cavity, an abdomino-pelvic cavity, a pelvic cavity, a vertebral cavity, and a cranial cavity. The methods described herein may treat or otherwise reduce a symptom of a metabolic disorder including one or more of obesity, Non-alcoholic fatty liver disease (NAFLD), Nonalcoholic steatohepatitis (NASH), proinflammatory processes, immunological processes, Alzheimer's disease, neurological disorders, Type I diabetes, and Type II diabetes, or another condition such as, for example, Barrett's esophagus and cancer.

In some variations, a method of treating tissue may comprise advancing a tissue treatment device, such as, for example, a pulsed electric field device, to a target tissue of a patient. For example, the method may comprise advancing a tissue treatment system and a visualization device to the target tissue of a patient. The tissue treatment system may optionally comprise a sheath and a tissue treatment device comprising an elongate body and an expandable member configured to treat tissue. For example, in some variations, the expandable member may include a treatment member such as, for example, an electrode array. In a delivery configuration, the expandable member may be disposed in the sheath circumferentially about the visualization device in an unexpanded configuration. The delivery configuration may be compact and facilitate cooperative (e.g., concurrent) advancement of the tissue treatment device and visualization device through a body lumen (e.g., esophagus, stomach, intestine) toward a target tissue site (e.g., duodenum). The expandable member may be advanced distal to the sheath while maintaining a position of the visualization device relative to the sheath. The expandable member may transition from the unexpanded configuration into an expanded configuration. For example, the expandable member in the expanded configuration may circumferentially contact tissue of a body cavity (e.g., duodenum). The target tissue may be treated using the tissue treatment device. For example, a pulsed electric field waveform may be delivered to the electrode array to generate a pulsed electric field to treat the tissue. Optionally, suction or negative pressure may be applied to increase apposition between the expandable member and/or the tissue treatment member and the tissue. After applying treatment with the expandable member (e.g., delivering energy, heat, cold, mechanical cutting), the expandable member may transition from the expanded configuration back into the unexpanded configuration. The expandable member may then be retracted into the sheath to reposition the system into the delivery configuration. In this manner, the tissue treatment device and visualization device may be translated together from a first tissue treatment site to a second tissue treatment site proximal or distal to the first tissue treatment site.

In variations in which a pulse electric field waveform is utilized, a first pulsed electric field waveform may be delivered to the electrode array to generate a first pulsed or modulated electric field, which may treat a first portion of target tissue. In some variations, the electrode array may have a plurality of sections. A first pulsed electric field waveform may be delivered to two or more non-proximate (e.g., non-adjacent, not immediately next to each other) sections of the plurality of sections in a predetermined sequence, which may increase safety and/or reduce unintended damage to the tissue by reducing a temperature increase in tissue. In some variations, the pulsed electric field device may be moved (e.g., advanced or retracted) toward a second portion of the target tissue (which may be distal or proximal to the first portion of the target tissue), and a second pulsed electric field waveform may be delivered to the electrode array to generate a second pulsed or modulated electric field thereby treating the tissue in the second portion. For example, in some variations, a signal generator may generate a drive voltage (e.g., voltage measured at an electrode array) of between about 400 V and about 1500 V that may correspond to an electric field strength of about 400 V/cm and about 7000 V/cm at the treatment portions of the duodenum. The expandable member may be in a compressed configuration, semi-expanded configuration, or an expanded configuration during movement of the pulsed electric field device. In some variations, sensor measurements (e.g., temperature, impedance) may be used to monitor and/or control pulse waveform delivery. In some variations, current and voltage measurements may be used to monitor and/or control pulse waveform delivery.

As described herein, an electrode array of an expandable member may be configured to maintain a predetermined electrode spacing as the expandable member changes dimensions, which may be facilitated by the manner in which the expandable member is manufactured. Also described herein is a method of manufacturing a tissue treatment device comprising the steps of disposing an electrode array on a surface of an expandable member where the electrode array may comprise a substrate comprising one or more apertures along a perimeter of the substrate. A bonding layer may be disposed over the electrode array. The electrode array may be bonded between the expandable member and the bonding layer using the apertures of the substrate.

### I. System

### Overview

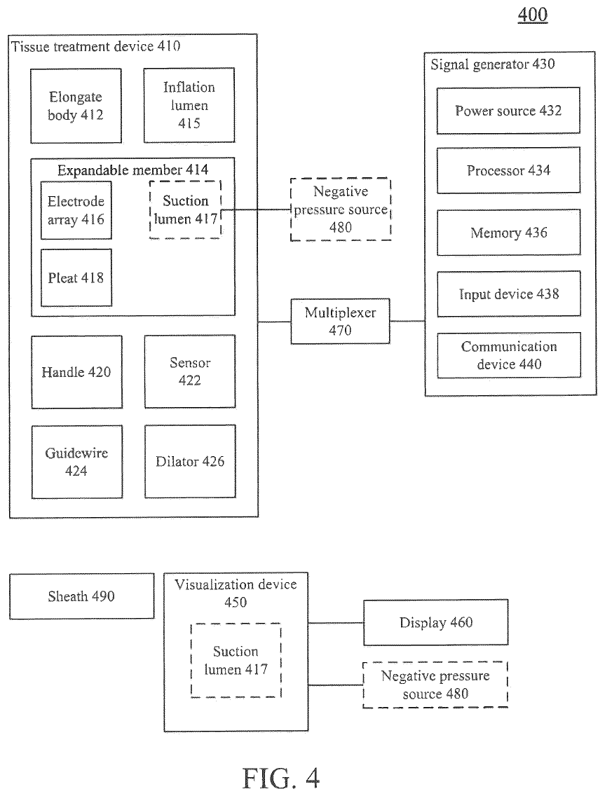

Systems described here may include one or more of the components used to treat tissue, such as, for example, a pulsed electric field device and a visualization device. Suitable examples of such systems and devices are described in International Application Serial No. PCT/US2022/025630, filed on Apr. 20, 2022, and U.S. Patent Application Ser. No. 63/563,149, filed on Mar. 8, 2024, the disclosure of each of which is hereby incorporated by reference in its entirety. FIG. 4 is a block diagram of a variation of a tissue treatment system (400) comprising one or more of a tissue treatment device (e.g., pulsed electric field device) (410), a signal generator (430), a visualization device (450), a display (460), a multiplexer (470), and a sheath (490) (e.g., delivery catheter). For example, the tissue treatment device (410) may be electrically coupled to the multiplexer (470) and signal generator (430). The sheath (490) may be configured to receive at least a portion of the tissue treatment device (410) and the visualization device (450). The visualization device may be coupled to the display (460).

In some variations, the tissue treatment device (410) may comprise one or more (e.g., a first and a second) elongate bodies (412) sized and shaped to be placed in one or more body cavities or lumens of the patient such as, for example, an esophagus, a stomach, a large intestine (e.g., cecum, colon, rectum, anal canal), a small intestine, any portion of the gastrointestinal tract, vasculature (e.g., blood vessels), a thoracic cavity (e.g., lungs), an abdomino-pelvic cavity, a pelvic cavity (e.g., bladder), a vertebral cavity, a cranial cavity (e.g., nasal passageway), and the like. In some variations, the tissue treatment device (410) may comprise one or more inflation lumens (415). In some variations, the elongate body (412) may be an overtube that defines a lumen configured to receive the visualization device (450). That is, the visualization device (450) may be disposed within the lumen of the elongate body (412) such that the tissue treatment device (410) and the visualization device (450) may be advanced together through one or more body cavities or lumens of the patient. In some variations, the elongate body (412) of the tissue treatment device (410) may be sized and shaped to be placed in a lumen of a sheath (490) concurrently with a visualization device (450) (e.g., alongside, visualization device disposed within elongate body).

In some variations, the tissue treatment device (410) may further comprise one or more expandable members (414) sized and shaped to at least partially engage tissue. Furthermore, the expandable member (414) may be sized and shaped to be placed in a sheath (490) concurrently with a visualization device (450). In some variations, the expandable member (414) may comprise one or more electrode arrays (416). In some variations, the expandable member (414) may further comprise one or more pleats (418) and/or one or more suction lumens (417). In some variations, the treatment device (410) may further comprise a handle (420), one or more sensors (422), a guidewire (424), and a dilator (426). A distal end of the tissue treatment device (410) may comprise the dilator (426), and the guidewire (424) may extend from a lumen of the elongate body (412), the dilator (426), and/or the sheath (490).

In some variations, the tissue treatment device may comprise a delivery configuration and a treatment configuration. The tissue treatment device may be placed in the delivery configuration when advanced to a predetermined tissue, when repositioned within a body cavity or lumen, and/or when removed from the body. Furthermore, the visualization device (450) may be disposed within a lumen of the elongate body of the treatment device (e.g., an overtube) and/or advanced through a window of the elongate body when the treatment device is placed in the delivery configuration. For example, in the delivery configuration, the expandable member (414) may be positioned circumferentially about the visualization device (450) within the sheath (490). The tissue treatment device may transition between the delivery and treatment configurations when, for example, the device is positioned at or near the predetermined tissue. For example, in the treatment configuration, at least a portion of the expandable member (414) may be extended distal to the sheath (490). In some variations, in the treatment configuration, the expandable member (414) may be placed in (e.g., transition to) one or more of an unexpanded and flattened configuration, an expanded configuration, and a partially expanded configuration.

The tissue treatment device may transition between the delivery and treatment configurations when, for example, the device is positioned at or near the target tissue (e.g., at a predetermined position relative to the target tissue). Furthermore, the visualization device may be advanced (e.g., passed, protruded) through a window of the elongate body (e.g., overtube) to visualize the configuration transitions of the expandable member. For variations including a sheath (490), at least a portion of the expandable member (414) may be extended distal to the sheath (490) in the treatment configuration. For variations including a window in the elongate body (412), at least a portion of the visualization device (450) may be advanced through the window and disposed outside the elongate body (412) in the treatment configuration. In some variations, in the treatment configuration, the expandable member (414) may be placed in (e.g., transitioned into) one or more of an unexpanded and flattened configuration, an expanded configuration, and a partially expanded configuration.

In some variations, the expandable member (414) in a treatment configuration may receive (e.g., be inflated with) any inert fluid, such as saline, contrast fluid, air, combinations thereof and the like. In some variations, the expandable member (414) may comprise a treatment member (416), such as, for example, an electrode array, a piezoelectric transducer, a laser, a blade, and a thermal element. For example, as will be described in more detail herein, in some variations the treatment member (416) may be coupled to a surface (e.g., outer surface, inner surface) of the expandable member (414), while in other variations, the treatment member (416) itself may form the expandable member (414) or the expandable member (414) may form the treatment member (416). For example, in some variations in which the treatment member (416) is an electrode array, the electrode array may form the expandable member (414). As another example, in variations in which the expandable member (414) may comprise a balloon containing heated or cooled fluid, the expandable member (414) may form the treatment member (416). In some variations, portions of the treatment member (416) and/or expandable member (414) may be activated or otherwise used to treat tissue individually. For example, in variations in which the treatment member (416) comprises an electrode array, the electrode array (416) may have a plurality of sections that may be energized individually (e.g., concurrently, consecutively) to treat tissue in a predetermined sequence as described in more detail herein. As another example, the treatment member (416) may comprise a fluid disposed within the expandable member (414) configured to provide a therapeutic effect (e.g., heated fluid-filled balloon, cryogenic fluid-filled balloon). In some variations, the expandable member (414) may be configured to translate relative to a distal end of the sheath (490), when employed, to transition between a delivery configuration and a treatment configuration, as described in more detail herein. Additionally or alternatively, the tissue treatment device (410) may comprise one or more sensors (422) configured to measure one or more predetermined characteristics of or near the target tissue, such as, for example, temperature, pressure, impedance and the like.

As mentioned above, the tissue treatment system (400) may comprise a visualization device (450). In some variations, the visualization device (450) may be configured to visualize one or more steps of a treatment procedure. The visualization device (450) may aid one or more of advancement of the tissue treatment device (410), positioning of the tissue treatment device (410), positioning of a suction lumen (417a), positioning of components of the tissue treatment device (e.g., the treatment member (416)), and confirmation of the treatment procedure. For example, the visualization device (450) may be configured to generate an image signal that is transmitted to a display (460), for example, on an output device. In some variations, the visualization device may be advanced together with and within a lumen of an elongate body (e.g., an overtube) (412) of the tissue treatment device (410). A distal portion of the elongate body (412) may define a window within a sidewall of the elongate body (412). The window may be configured to receive the visualization device (450) therethrough such that the visualization device (450) may advance out of the lumen, through the window, and into a space outside of the elongate body (412) and proximal to the expandable member (414). Alternatively, the visualization device (450) may be advanced separately from, such as, for example, alongside the tissue treatment device (410), and relative to the sheath (490). For example, the sheath (490) may be configured to concurrently receive the expandable member (414) and the visualization device (450) such that the tissue treatment device (410) translates together with the visualization device (450) within a lumen of the sheath (490). In some variations, the sheath (490) may be configured to receive the expandable member (414) of the tissue treatment device (410) and a distal portion of the visualization device (450). The expandable member (414) may advance from a distal end of the sheath (490) while a position of the visualization device (450) may be maintained relative to the sheath (490), thereby enabling freedom of movement for the expandable member (414). In some variations, the expandable member (414) may be translatable and/or rotatable with respect to the sheath (490) and/or the visualization device (450). In some variations, the visualization device (450) may be translatable with respect to the sheath (490). Alternatively, in some variations, the visualization device (450) may be integrated with the sheath (490). For example, a distal end of the visualization device (450) may comprise the sheath (490).

The visualization device (450) may be any device (internal or external to the body) that assists a user in visualizing a treatment procedure. In some variations, the visualization device (450) may comprise one or more of an endoscope (e.g., chip-on-the-tip camera endoscope, three camera endoscope), image sensor (e.g., CM OS or CCD array with or without a color filter array and associated processing circuitry), camera, fiberscope, external light source, and ultrasonic catheter. In some variations, an external light source (e.g., laser, LED, lamp, or the like) may generate light that may be carried by fiber optic cables. Additionally or alternatively, the visualization device (450) may comprise one or more LEDs to provide illumination. For example, the visualization device (450) may comprise a bundle of flexible optical fibers (e.g., a fiberscope). The bundle of fiber optic cables or fiberscope may be configured to receive and propagate light from an external light source. The fiberscope may comprise an image sensor configured to receive reflected light from the tissue and the pulsed electric field device. It should be appreciated that the visualization device (450) may comprise any device or devices that allows for or facilitates visualization of any portion of the pulsed electric field device and/or of the internal structures of the body. For example, the visualization device may comprise a capacitive sensor array and/or a fluoroscopic technique for real-time X-ray imaging.

In some variations, the tissue treatment device (410) may comprise a suction lumen (417) configured to apply suction to the expandable member (414) and tissue. For example, the suction lumen (417) may be slidably positioning within, and advanced from, a lumen of the visualization device (450). In some variations, the suction lumen (417) may correspond to a lumen of the visualization device (450). The suction lumen (417) may fluidically couple to the expandable member (414) in an expanded configuration while the visualization device (450) is positioned proximally of the expandable member (414). Alternatively, the suction lumen (417) may correspond to a suction catheter separate from the visualization device (450) and tissue treatment device (410). In some variations, the suction lumen (417) may be fluidically coupled to a negative pressure source (480).

Generally, treatment source (430) may be configured to facilitate treatment of tissue using the treatment member (416). For example, the treatment source (430) may comprise a signal generator configured to provide energy (e.g., energy waveforms, pulse waveforms) to the tissue treatment device (410) to treat predetermined portions of tissue, such as, for example, duodenal tissue. As another example, the treatment source (430) may comprise a fluid source configured to provide fluid (e.g., heated fluid, cryogenic fluid) to the tissue treatment device (410) to treat predetermined portions of tissue. In some variations, a PEF system as described herein may include a signal generator that comprises an energy source and a processor. The signal generator may be configured to deliver a bipolar waveform to an electrode array, which may deliver energy to the tissue (e.g., duodenal tissue). The delivered energy may aid in resurfacing or otherwise treating the desired tissue while minimizing damage to surrounding tissue. In variations in which the desired tissue is duodenal tissue, the delivered energy may aid in resurfacing the mucosa of the duodenum while minimizing damage to surrounding tissue (e.g., muscularis tissue). In some variations, the signal generator may generate one or more bipolar waveforms.

In some variations, in order to limit nerve stimulation, a pulse waveform may, on average, comprise a net current of about zero (e.g., generally balanced positive and negative current), and have a non-zero time of less than about 2 μsec or less than about 5 μsec. In some variations, the pulse waveform may comprise a square or rectangular waveform. For example, the pulse waveform may comprise a square or rectangular shape in voltage drive and in current drive, or the pulse waveform may comprise a square or rectangular shape in voltage drive and a sawtooth shape in current drive. In some variations, one or more pulses may comprise a half sine-wave for both current and voltage. In some variations, one or more pulses may comprise two exponentials with different rise and fall times. In some variations, one or more pulses may comprise bipolar pulse at a first potential followed by pulse pairs at a second potential less than the first potential.

In some variations, a multiplexer (470) may be coupled to the tissue treatment device (410). For example, the multiplexer (470) may be coupled between the signal generator (430) and the tissue treatment device (410), or the signal generator (430) may comprise the multiplexer (470). The multiplexer (470) may be configured to select a subset of electrodes of an electrode array (416) receiving a pulse waveform generated by the signal generator (430) according to a predetermined sequence. For example, in some variations, the electrode array (416) may comprise one or more sections that correspond to a subset of electrodes. The electrode array (416) may comprise between 1 and 10 sections, including 1, 2, 3, 4, 5, 6, 7, 8, 9, or 10 sections. Each section may comprise the same number of electrodes and/or the same surface area as every other section, but need not. The predetermined sequence may be optimized to treat tissue at a given treatment site. Additionally or alternatively, the multiplexer (470) may be coupled to a plurality of signal generators and may be configured to select between a waveform generated by one of the plurality of signal generators (430) for a selected subset of electrodes.

In some variations, the multiplexer (470) and the signal generator (430) may be configured to deliver a pulsed electric field waveform to two or more non-proximate sections (e.g., first section, second section) of the plurality of sections in a predetermined sequence. For example, the predetermined sequence may comprise activating a first section followed by a second section after an inter-section delay where activation of each of the first and second sections generates a therapeutic electric field, and where the first and second sections are not adjacent (i.e., directly next to) one another. Put differently, the predetermined sequence may comprise activating a first section followed by a second section, where at least a third section is positioned between the first and second sections. As another example, the signal generator may be configured to deliver a series of bipolar pulses to two or more non-proximate sections of the plurality of sections in a predetermined sequence for a cumulative activation time of between about 0.1 ms and about 10 ms over a treatment period between about 30 seconds and about 35 seconds. Each bipolar pulse may comprise a pulse width between about 1 μs and about 10 μs, and the electrode array may be configured to deliver between about 0.05 J per bipolar pulse and about 0.5 J per bipolar pulse and an instantaneous power between about 26,000 W per bipolar pulse and about 70,000 W per bipolar pulse. In some variations, the predetermined sequence may comprise a duty cycle between about 0.003% and about 0.004%, including all ranges and sub-values in-between. In some variations, the signal generator may be configured to control waveform generation and delivery in response to received sensor data. For example, energy delivery may be modulated (e.g., inhibited) based on one or more of a measured temperature and impedance.

### Tissue Treatment System

Generally, the tissue treatment systems described herein may comprise a tissue treatment device coupled to a treatment source and a separate visualization device configured to provide visualization of the tissue treatment device and tissue. The tissue treatment devices described herein may comprise an elongate body (e.g., overtube) and an expandable member comprising or otherwise forming a treatment member (e.g., an electrode array). The tissue treatment devices may be configured to facilitate deployment to, and treatment of, target tissue such as tissue within a body cavity or lumen such as the duodenum. For example, in some variations, the tissue treatment device may be a pulsed electric field device configured to apply pulsed or modulated electric field energy to an inner surface or circumference of the body cavity or lumen. The devices described herein may be used to treat only a particular, pre-specified portion (e.g., 5%, 10%, 15%, 20%, 25%, 30% of a body cavity or lumen, duodenum, esophagus), and/or an entirety of the body cavity or lumen (e.g., the entire length of the duodenum, the entire length of the esophagus). Additionally or alternatively, the devices described herein may be used to treat one or more of a duodenum, a pylorus, an esophagus, a stomach, a small intestine, and a large intestine (e.g., cecum, colon, rectum, anal canal), as well as any body cavity or lumen of the patient such as vasculature (e.g., blood vessels), a thoracic cavity (e.g., lungs), an abdomino-pelvic cavity, a pelvic cavity (e.g., bladder), a vertebral cavity, a cranial cavity (e.g., nasal passageway), and the like. The treated tissue may treat and/or reduce one or more symptoms of one or more of a metabolic disorder, pre-cancer, cancer, proinflammatory processes, immunological processes, Alzheimer's disease, and neurological disorders. For example, the metabolic disorder may comprise one or more of obesity, non-alcoholic fatty liver disease (NAFLD), nonalcoholic steatohepatitis (NASH), Type I diabetes, and Type II diabetes. The treated tissue may treat and/or reduce one or more symptoms of Barrett's esophagus. For example, the metabolic disorder may comprise one or more of obesity, non-alcoholic fatty liver disease (NAFLD), nonalcoholic steatohepatitis (NASH), Type I diabetes, and Type II diabetes.

In some variations, an electrode array of a pulsed electric field device may generate an electric field strength of from about 400 V/cm to about 1500 V/cm, from about 1500 V/cm to about 4500 V/cm, including all values and sub-ranges in-between, at a treatment depth of from about 0.5 mm to about 1.5 mm from an inner surface of the duodenum, for example, at about 1 mm. For example, in some variations, the pulsed electric field may comprise an electric field strength (i.e., magnitude) between about 2000 V/cm to about 4500 V/cm, about 3000 V/cm to about 4500 V/cm, about 3500 V/cm to about 4500 V/cm, about 3750 V/cm to about 4250 V/cm, or about 3900 V/cm to about 4100 V/cm, including about 2000 V/cm, about 3000 V/cm, about 3500 V/cm, about 3750 V/cm, about 3900 V/cm, about 4000 V/cm, about 4100 V/cm, about 4250 V/cm, and about 4500 V/cm.

In some variations, the electric field may decay such that the electric field strength is less than about 400 V/cm at about 3 mm from the inner surface of the duodenum. In some variations, a predetermined bipolar current and voltage sequence may be applied to an electrode array of the pulsed electric field device to generate the pulsed or modulated electric field. The generated pulsed or modulated electric field may be substantially uniform to robustly induce cell lysis in a predetermined portion of duodenal tissue. For example, a generated pulsed or modulated electric field may spatially vary up to about 20% at a predetermined depth of tissue, between about 5% and about 20%, between about 10% and 20%, and between about 5% and about 15%, including all ranges and sub-values in-between. Furthermore, the pulsed electric field device may be biocompatible and resistant to stomach acids and intestinal fluids.

In some variations, a tissue treatment system may comprise a pulsed electric field device configured to treat tissue (e.g., regenerate mucosal and submucosal cells of the duodenum). The pulsed electric field device in a delivery configuration (e.g., an expandable member of the pulsed electric field device) may be disposed within a sheath and releasably coupled to a visualization device (e.g., endoscope), such as, for example, to an outer surface of the visualization device. The pulsed electric field device may comprise an expandable member such as an inflatable balloon having an electrode array coupled thereto. The expandable member and the electrode array may be advanced distal to the visualization device and transitioned to a treatment configuration when the system is located at a tissue treatment site. The expandable member may be positioned and inflated to create apposition between the electrode array and to the tissue. The electrode array may generate a pulsed electric field to treat the tissue.

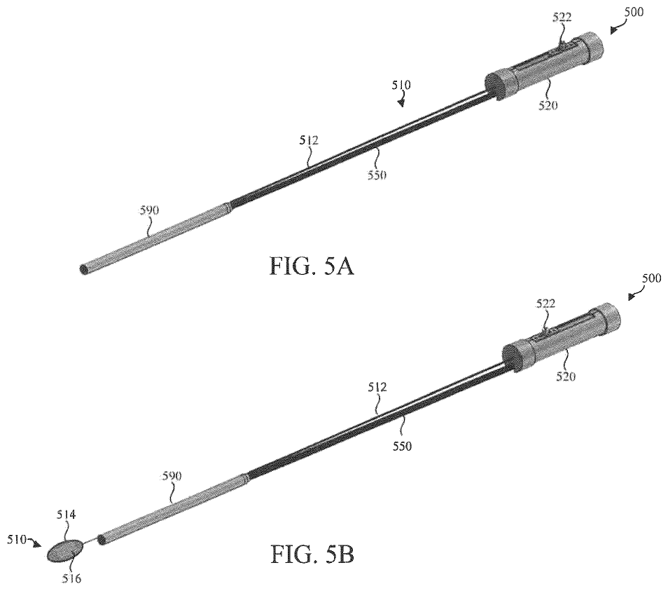

FIGS. 5A and 5B are perspective views of an illustrative variation of a tissue treatment system (500) in respective delivery and treatment configurations. FIG. 5A shows the system (500), and in particular the expandable member (514), in a delivery configuration where a distal end of the tissue treatment device (510) and the visualization device (550) are disposed (e.g., housed, constrained) within a lumen of the sheath (590). In the delivery configuration, the expandable member (514) may have a shape and size that fits with the visualization device (550) within a lumen of the sheath (590). Put differently, a lumen of the sheath (590) may have an inner diameter sized and shaped to receive at least a portion of the visualization device (550) and the expandable member (514) concurrently. For example, in a delivery configuration, the expandable member (514) may be disposed between an inner surface of the sheath (590) and an outer surface of the visualization device (550). For example, the expandable member (514) may be rolled, wrapped, folded, overlapped, wound about, spooled, housed, and/or constrained between an inner surface of the sheath (590) and an outer surface of the visualization device (550). That is, the expandable member (514) may be in mechanical contact with at least a portion of the visualization device (550). While the tissue treatment device in FIGS. 5A and 5B is shown and described as a pulsed electric field device, it should be appreciated that the tissue treatment device may utilize a different treatment member (instead of or in addition to an electrode array) to treat tissue utilizing a different treatment modality as described herein, and/or may utilize an electrode array to apply energy in a different therapeutic manner, such as, for example, using heat-based treatment modalities (e.g., ablation).

In some variations, each of the tissue treatment device (510) and the visualization device (550) may be moveable (e.g., translatable and/or rotatable) with respect to the sheath (590). Alternatively, in some variations, the visualization device (550) may be fixedly coupled to the sheath (590) such that it cannot translate and/or rotate relative to the visualization device (550). For example, a proximal portion of the sheath (590) may be fixedly coupled to the visualization device (550) and/or a distal portion of the visualization device (550) may comprise (e.g., be integrated with) the sheath (590). In some variations, the sheath (590) may have a length at least about equal (e.g., equal) to a length of the expandable member (514) in the delivery configuration. Additionally or alternatively, the sheath (590) may extend up to a proximal end of the elongate body (510) and/or a proximal end of the visualization device (550). As described in more detail herein, the expandable member (514) may be positioned circumferentially about the visualization device in the delivery configuration. In some variations, a diameter of the system in the delivery configuration may not exceed about 15 mm, about 17 mm, about 20 mm, about 25 mm, and about 30 mm, including all ranges and sub-values in-between. For example, a distal portion of the system (500) including the expandable member (514) and the visualization device (550) disposed within the sheath (590) may not exceed a combined diameter of about 15 mm, about 17 mm, about 20 mm, about 25 mm, and about 30 mm, including all ranges and sub-values in-between. In some variations, a rigidity of the sheath (590) may be greater than a rigidity of either of, including both of, the elongate body (512) and the visualization device (550).

FIG. 5B shows the system (500) in a treatment configuration where the expandable member (514) is advanced distal to the sheath (590) and the visualization device (550). The expandable member (514) in FIG. 5B is further shown in a flattened configuration having a width larger than an inner diameter of the sheath (590). The expandable member (514) shown in FIGS. 5A and 5B is in an unexpanded (e.g., uninflated) configuration and may be expanded (e.g., inflated) in the treatment configuration. The expandable member (514) may comprise an electrode array (516) configured to generate a pulsed electric field. The expandable member (514) may be coupled to a distal portion of the elongate body (512).

In some variations, a handle (520) may be coupled to a proximal portion of the elongate body (510) and the visualization device (550). In some variations, the handle (520) may comprise an actuator (522) configured to control one or more of the expandable member (514) and the visualization device (550). For example, the actuator (522) may include a slider configured to translate (e.g., advance, retract) the expandable member (514) relative to the sheath (590). In some variations, the handle (520) may be electrically connected to a signal generator (430). While depicted with a single handle (520), it should be appreciated that in some variations, one or more additional handles may be utilized to control components of the system, such as, for example, the visualization device.

Although not depicted in FIGS. 5A and 5B, the tissue treatment system (500) may comprise additional elongate bodies configured to provide one or more of inflation, suction, electrical power, and the like. For example, one or more of a second elongate body comprising an inflation lumen, a third elongate body comprising a suction lumen, and a fourth elongate body comprising a lead wire may be provided separately from the first elongate body (512) coupled to the expandable member (514). Any of, including all of, the second, third, and fourth elongate bodies may be independently disposed within the sheath (590) or may be positioned over an outer surface of the sheath (590). In some variations, a single elongate body (512) may comprise one or more of an inflation lumen, a suction lumen, a lead wire, a pull wire, and the like. In some variations, a pull wire may be configured to deflect one or more of the elongate body (512) and the expandable member (514). In some variations, a series of expandable members (514) may be coupled to a distal portion of the elongate body (512). For example, the expandable members (514) may be arranged in a serial manner (e.g., in a daisy chain). In some variations, the elongate body (512) and the visualization device (550) may be mechanically coupled (e.g., wrapped, clipped) to each other at one or more locations between the sheath (590) and the handle (520).

In some variations, one or more sensors may be coupled to one or more of the tissue treatment device (510), the visualization device (550), and the sheath (590). For example, one or more of the tissue treatment device (510), the visualization device (550), and the sheath (590) may comprise one or more of a temperature sensor and a pressure sensor, and a sheath (590) may comprise one or more of a pressure sensor and an impedance sensor.

In some variations, the tissue treatment device (510) may be a pulsed electric field device configured to treat tissue (e.g., regenerate mucosal and submucosal cells of the duodenum). The pulsed electric field device in a delivery configuration (e.g., an expandable member of the pulsed electric field device) may be disposed within the sheath and releasably coupled to a visualization device (e.g., endoscope), such as, for example, to an outer surface of the visualization device. The pulsed electric field device may comprise an expandable member such as a balloon having an electrode array coupled thereto. The expandable member and the electrode array may be advanced distal to the visualization device and transitioned to a treatment configuration when the system is located at a tissue treatment site. The expandable member may be positioned and inflated to create apposition between the electrode array and to the tissue. The electrode array may generate a pulsed electric field to treat the tissue.

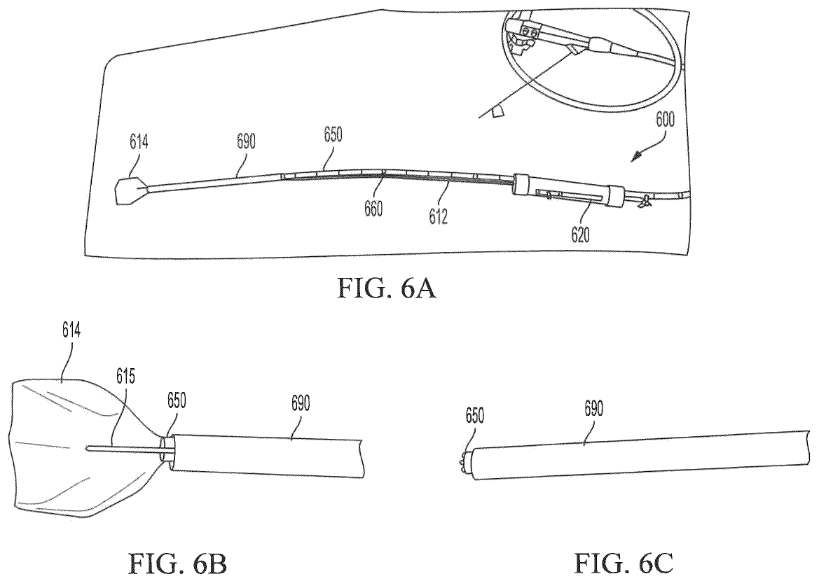

FIG. 6A is an image of a side view of an illustrative variation of a tissue treatment system (600) in a treatment configuration. The tissue treatment system (600) may comprise an elongate body (612) coupled to an expandable member (614), a visualization device (650) (e.g., endoscope), a sheath (690), and a handle (620). The sheath (690) may comprise a lumen configured to receive the elongate body (612), expandable member (614), and visualization device (650). The handle (620) may be coupled to a proximal portion of each of the elongate body (612) and the visualization device (650). A proximal portion of the sheath (690) may be mechanically coupled (e.g., fixedly attached) to the visualization device (650). FIG. 6A depicts the expandable member (614) in a flattened, unexpanded configuration. In some variations, one or more fasteners (660) (e.g., clip, loop), may be configured to couple the elongate body (612) to the visualization device (650). FIG. 6B is a detailed side view of the tissue treatment system (600) in the treatment configuration where the expandable member (614) is distal to a distal end of the sheath (690). For example, the elongate body (612) may be configured to translate the expandable member (614) relative to the sheath (650). In some variations, an inflation lumen (615) may be fluidically coupled to the expandable member (614) to facilitate expansion/flattening (e.g., inflation/deflation) of the expandable member (614). The inflation lumen (615) may be configured to receive a fluid (e.g., saline) to inflate the expandable member (614). In some variations, the elongate body (612) may be coupled to a sidewall of the expandable member (614). The expandable member (614) in FIGS. 6A and 6B does not include an electrode array to better illustrate the expandable member and the inflation lumen (615). In some variations, the expandable member (614) may be substantially transparent. FIG. 6C is a detailed side view of the tissue treatment system (600) in a delivery configuration where the expandable member (614) is substantially withdrawn into a lumen of the sheath (690) such that a diameter of the system (600) corresponds to an outer diameter of the sheath (690). In some variations, a distal end of the visualization device (650) may be generally aligned with a distal end of the sheath (690) in one or both of the delivery and treatment configurations. For example, the distal end of the visualization device (650) may be aligned with, or disposed slightly distal (as shown in FIGS. 6B and 6C, within about 10% length of the expandable member) or proximal to a distal end of the sheath (690). In some variations, the expandable member (614) may comprise a proximal taper. The configuration of the expandable member is further discussed herein.

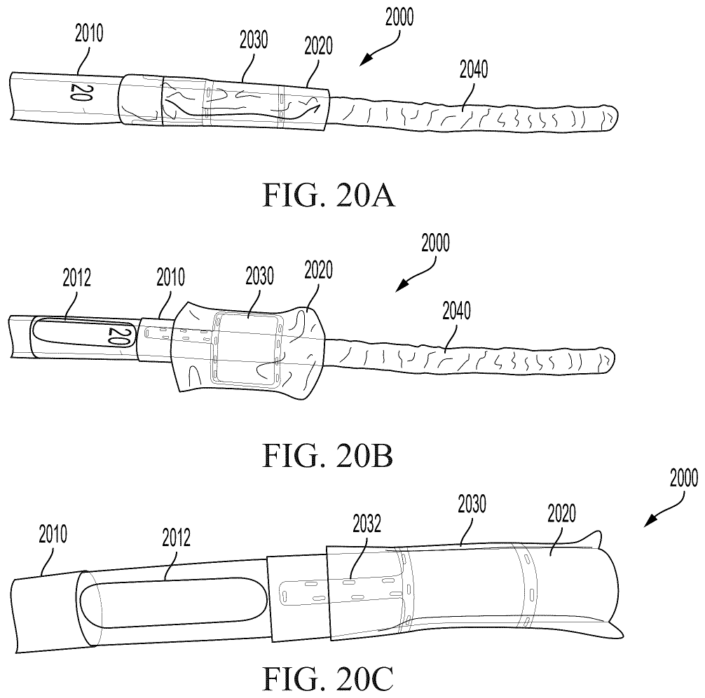

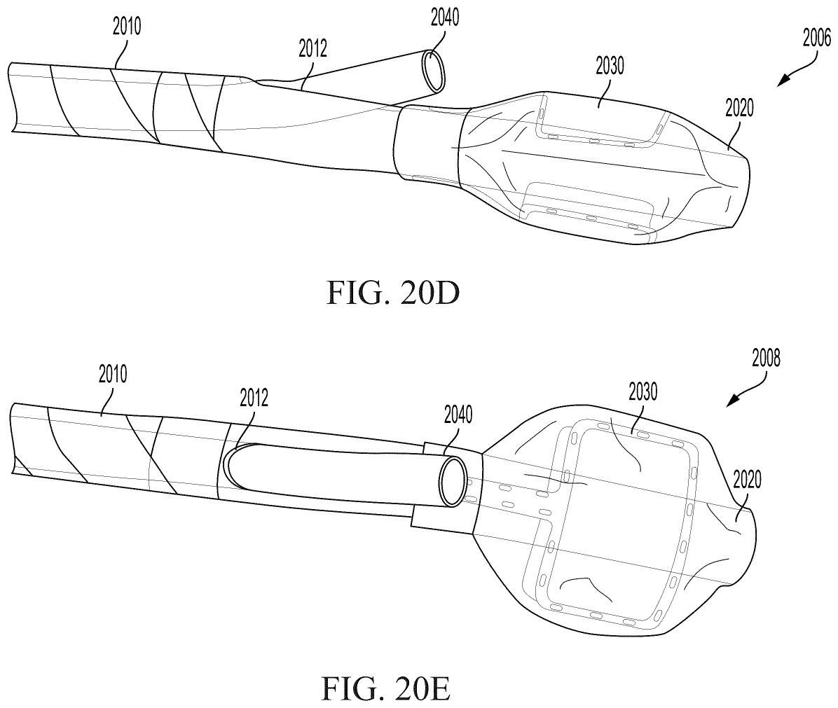

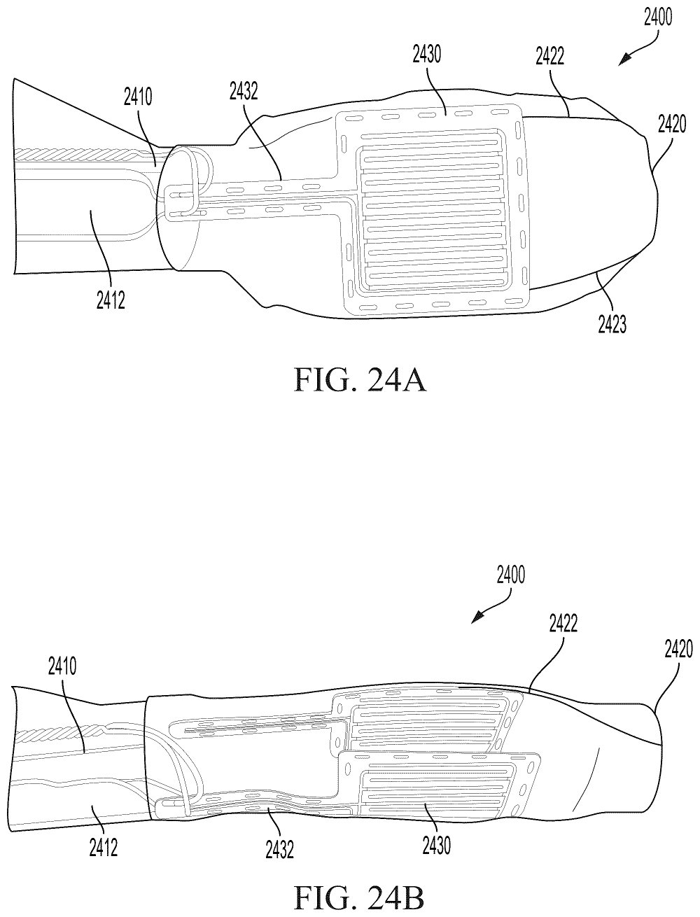

Another variation of a tissue treatment system including a visualization device disposed within a lumen of a tissue treatment device as described in detail below with respect to FIGS. 20A-20E and 24A-24B. FIGS. 20A and 20B are images of a side view and top view of an illustrative variation of a visualization device (2040) advanced through an elongate body, in the form of an overtube (2010), and an expandable member (2020) (e.g., balloon) of a tissue treatment device (2000). FIGS. 20A and 20B show the device (2000), and in particular the expandable member (2020), in a delivery configuration (e.g., flat configuration) where a distal end of the visualization device (2040) is advanced distal to the overtube (2010) and expandable member (2020) and through a lumen of the overtube (2020) and expandable member (2020). In this configuration, the tissue treatment device (2000) and visualization device (2040) may be advanced together (e.g., simultaneously) through the patient, thereby reducing or eliminating the need for fluoroscopic imaging. A lumen of the overtube (2010) and a lumen of the expandable member (2020) may have a diameter sized and shaped to receive at least a portion of the visualization device (2040) such that the visualization device (2040) may translate (e.g., advance, withdraw) through one or more of the overtube (2010) and the expandable member (2020). A proximal portion of the expandable member (2020) may be coupled to a distal portion of the overtube (2010). In some variations, a rigidity of the overtube (2010) may be less than a rigidity of the visualization device (2040), which may enhance steerability of the tissue treatment device (2000) as the system is delivered to a target treatment site (e.g., target tissue). One or more handles (not shown) may be coupled to one or more of a proximal portion of each of the overtube (2010) and the visualization device (2040).

In order for the tissue treatment system to advance through one or more body cavities such as, for example, the esophagus, the tissue treatment system must be relatively compact in diameter. For example, in some variations, an outermost diameter of the tissue treatment system in the delivery configuration may not exceed about 15 mm, about 17 mm, about 20 mm, about 25 mm, or about 30 mm, between about 15 mm and about 30 mm, about 17 mm and about 25 mm, about 20 mm and about 25 mm, about 15 mm and about 20 mm, about 15 mm and about 17, including all ranges and sub-values in-between. For example, a distal portion of the device (2000) including the expandable member (2020) and the visualization device (20400) disposed within the overtube (2010) and/or expandable member (2020) may not exceed a combined diameter of about 15 mm, about 17 mm, about 20 mm, about 25 mm, and about 30 mm, including all ranges and sub-values in-between. FIG. 20C shows the device (2000) in a delivery configuration (e.g., without a visualization device (2040)) where the expandable member (2030) is in a flattened, unexpanded, and uninflated configuration. Furthermore, the expandable member (2030) may be wound around its own circumference and temporarily held in place (e.g., using one or more of an adhesive, tape, suture, heat formed/set shape, and the like) until the expandable member (2030) is transitioned to an expanded, treatment configuration.

In some variations, the expandable member (2020) may comprise a treatment member configured for treating tissue. For example, the treatment member may comprise an electrode array (2030) configured to generate a therapeutic electric field (e.g., pulsed electric field). As described in more detail herein, the expandable member (2030) may include one or more pleats (e.g., two, three, four, or more) configured to transition the balloon between a folded configuration having a first diameter and an unfolded configuration having a second diameter greater than the first diameter. The one or more pleats may facilitate radial expansion of the expandable member while allowing the treatment member (e.g., electrode array) to maintain its structural integrity (e.g., electrode spacing).

In some variations, one or more sensors may be coupled to one or more of the overtube (2010), the visualization device (2040), and the expandable member (2020). For example, one or more of the overtube (2010), the visualization device (2040), and the expandable member (2020) may comprise one or more of a temperature sensor, a pressure sensor, and an impedance sensor.

FIGS. 20D and 20E are images (2006, 2008) of an illustrative variation of a tissue treatment system in a treatment configuration where the expandable member (2020) is in an expanded configuration. For example, after advancing the tissue treatment device (2000) and the visualization device (2040) to a treatment site, the tissue treatment device (2000) may be advanced over the visualization device (204) (e.g., the visualization device being held in place) such that the window (2012) of the overtube (2010) is distal to the visualization device (2040). Then, the visualization device (2040) may be advanced through the window (2012) of the overtube (2010) as shown in FIG. 20D such that a distal end of the visualization device (2040) is external to the tissue treatment device (2000). As shown in FIGS. 20D and 20E, the visualization device (2040) advanced through the window (2012) may be proximal to the expandable member (2020) such that one or more of the expandable member (2040) and target tissue (e.g., ampulla, duodenum, esophagus) may be visualized. For example, the visualization device (2040) may visualize the expandable member (2020) when: transitioning between an unexpanded configuration and an expanded configuration; treating (and re-treating) one or more portions of tissue; and repositioning the tissue treatment device. In some variations, an inflation lumen (not shown) may be fluidically coupled to the expandable member (2020) to facilitate expansion/flattening (e.g., inflation/deflation) of the expandable member (2020). For example, the inflation lumen may be configured to receive a fluid (e.g., saline or air) to inflate the expandable member (2020). In some variations, one or more of the overtube (2010) and the expandable member (2020) may be substantially transparent to facilitate visualization. Furthermore, the visualization device (2040) may be configured to apply suction to a portion of the target tissue through a lumen (e.g., working channel) of the visualization device (2040). Additionally or alternatively, a suction lumen (not shown) of the overtube (2010) may be configured to apply suction to the tissue.

FIGS. 24A and 24B are images of a top view and side view of an illustrative variation of a tissue treatment device (2400) including an overtube (2410) coupled to an expandable member (2420), and an electrode array (2430) coupled to the expandable member (2420). The overtube (2410) may comprise a window (2412) configured to receive a visualization device (not shown) therethrough. In some variations, the overtube may comprise a plurality (e.g., two, three, four or more) of windows that may be disposed axially or radially about the overtube. The electrode array (2430) may comprise one or more lead wires (2432) coupled to a signal generator (not shown). The expandable member (2420) may comprise one or more pleats (2422, 2423). FIG. 24A shows the expandable member (2420) in a flattened (e.g., unexpanded) configuration where the pleats (2422, 2423) are folded inward and form a proximal taper of the expandable member (2020). The pleats (2422, 2423) may be disposed along one or more lateral portions of the expandable member (2420) (e.g., parallel to a longitudinal axis of the expandable member (2420)). FIG. 24A shows a first pleat (2422) on a first side of the expandable member (2420) and a second pleat (2433) on a second side of the expandable member (2420) opposite (e.g., 180 degrees from) the first side. In the folded configuration (FIG. 24A), the expandable member (2420) may have a first diameter, and in an unfolded configuration (FIG. 24B), the expandable member (2420) may have a second diameter greater than the first diameter. For example, FIG. 24B shows coil

folded pleat (2422) extending laterally outward from a longitudinal axis of the expandable member (2420). In some variations, the expandable member may comprise a plurality (e.g., two, three, four or more) of pleats where the pleats are spaced apart circumferentially by about 30 degrees, about 60 degrees, and 90 degrees, about 120 degrees, about 180 degrees, about 270 degrees, about 90 degrees to about 120 degrees, about 30 degrees to about 60 degrees, about 60 degrees to about 90 degrees, about 90 degrees to about 120 degrees, or about 30 degrees to about 270 degrees, including all ranges and sub-values therebetween. The pleats 2422 may, or may not, comprise one or more sections of the electrode array.

### Expandable Member

Generally, the expandable members described herein may be configured to transition between delivery and treatment configurations to facilitate delivery with a visualization device through a patient body and aid in positioning the electrode array relative to target tissue during a treatment procedure. For example, the expandable member (e.g., inflatable member such as an inflatable balloon) may expand to contact tissue. In some variations, contact between the expandable member and the tissue may hold, or assist in holding, the pulsed electric field device (e.g., elongate body, electrode array, sensor) in place relative to the tissue without catching or trapping tissue. As described above, the expandable member may comprise an electrode array. The electrode array may be or otherwise comprise any of the electrode arrays described herein. In some variations, such as, for example, when the expandable member comprises an exemplary inflatable balloon, the electrode array may be disposed on an outer surface of the expandable member. In other variations, the electrode array may be disposed on an inner surface of the expandable member. In other variations, the electrode array may be disposed within a wall of the expandable member. In some variations, the expandable member may comprise a lumen therethrough configured to receive the visualization device. For example, the visualization device may be advanced through the lumen of the expandable member and disposed distal to the expandable member to facilitate movement of the system through a body lumen and visualization of tissue and/or other components of the system (e.g., the expandable member) without the need for fluoroscopic guidance.

In some variations, the expandable member may comprise one or more of an inflatable balloon, a stent, a scaffold, a support, a basket, a frame, and/or a cage. In each of these variations, the electrode array is supported by and/or disposed on, or along, the expandable member. In some variations, the stent, scaffold, support, basket, frame and/or cage may comprise a shape memory material such as nitinol or the like. In these variations, the stent, scaffold, support, basket, frame and/or cage may be collapsed in a deformed, collapsed configuration within a sheath, or delivery catheter. When released by, e.g, distal translation out of the sheath, or delivery catheter, the stent, scaffold, support, basket, frame and/or cage may expand to achieve an expanded configuration via the shape memory material to engage the target tissue for subsequent treatment with the electrode array that is disposed on or along the stent, scaffold, support, basket, frame and/or cage. Repositioning to treat a second target tissue may be achieved by retracting the expanded stent, scaffold, support, basket, frame and/or cage in to the sheath, or delivery catheter, translating the device or system to the second target tissue, where the stent, scaffold, support, basket, frame and/or cage may be translated distally out of the sheath, or delivery catheter, to expand and engage the second target tissue for treatment.

Expandable stents, scaffolds, supports, baskets, frames and/or cages are well known in the art. See, for example, U.S. Pat. No. 5,133,732 for “Intravascular Stent” and U.S. Pat. No. 5,263,963 for “Expandable cage catheter for repairing a damaged blood vessel”, the entire contents of each of which are hereby incorporated by reference.

The expandable member may comprise a delivery configuration and a treatment configuration. In the delivery configuration, the expandable member is in an unexpanded configuration (e.g., uninflated configuration) to facilitate placement of the expandable member between an inner surface of a sheath and an outer surface of a visualization device. In the treatment configuration, the expandable member may transition between the unexpanded configuration and an expanded configuration (e.g., inflated configuration), as well as a state in-between the unexpanded and expanded configurations (e.g., partially inflated). Placing the expandable member in the unexpanded delivery configuration may allow the pulsed electric field device to be compact in size, which may facilitate placement together with a visualization device within a lumen of a sheath having a constrained diameter. Once the system has been appropriately positioned near a target tissue treatment site, the expandable member may be advanced to be unconstrained by a sheath and transitioned to a treatment configuration. For example, the expandable member may be advanced distally, at least partially beyond a distal end of a sheath and then transitioned to a treatment configuration. An unconstrained expandable member may be biased to unwrap (e.g., unfold, unroll) into a flattened configuration as the expandable member is advanced. The expandable member in the flattened configuration may be unexpanded (e.g., uninflated) and may transition to an expanded configuration (e.g., inflated configuration) where a size and volume of the expandable member increases to allow an electrode array of the expandable member to facilitate contact or better contact a tissue surface, such as, for example, all or a portion of an inner circumference of a body lumen such as the duodenum. Conversely, from the treatment configuration, the expandable member may be repositioned in the sheath and transitioned to the delivery configuration. The expandable member may be transitioned to the delivery configuration from the expanded configuration or the unexpanded (e.g., flattened) configuration. For example, the expandable member in the expanded configuration may be unexpanded (e.g., deflated) and flattened (e.g., via natural bias), and then transitioned into the delivery configuration when retracted into the sheath. As discussed above, the expandable member may comprise one, or more than one, inflatable balloon.

The geometry, dimensions, material, and properties of the expandable member may provide strength to inflate and provide apposition to tissue while also having sufficient flexibility to reproducibly be positioned circumferentially between a sheath and visualization device roll and unroll without becoming entangled with itself or tissue. In some variations, the expandable member may be composed of a material biased to form to a predetermined shape. For example, the expandable member may comprise one or more of a flexible polymeric material (e.g., polyamide, PET), a thermoplastic urethane, thermoplastic elastomer, polyethylene terephthalate, polyimide, nylon, biaxially-oriented polyethylene terephthalate, nitinol, combinations thereof, and the like. In some variations, the expandable member may comprise a support member configured to add stiffness and/or strength in a treatment configuration. For example, the support member may allow an electrode array of the expandable member to improve apposition (e.g., engagement, contact) with a tissue surface. Additionally or alternatively, the support member may be configured to add stiffness and/or strength to the expandable member to facilitate transitions between a delivery configuration and treatment configuration. For example, the support member may provide axial stiffness when pushing the expandable member relative to the sheath and/or visualization device from the delivery configuration to the treatment configuration. In some variations, a portion of the expandable member comprising the treatment member (e.g., an electrode array) may comprise a first rigidity, and one or more other portions of the expandable member may comprise a second rigidity different than the first rigidity, which may facilitate expansion and contraction of different portions of the expandable member at different rates. For example, in variations in which the treatment member comprises an electrode array, a predetermined spacing between the electrodes of the plurality of electrodes of the array may be maintained when the expandable member is expanded from an unexpanded configuration to a partially or fully expanded configuration, such, for example, in variations in which the devices and systems described herein are used in a duodenum of a patient, a diameter of between about 15 mm and about 45 mm. Put differently, the electrode geometry and/or the spacing between the electrodes of the array may remain constant as the expandable member is expanded to any value within a predetermined range associated with a range of diameters necessary or desirable to provide treatment patients with a variety of target lumen diameters. In this manner, the devices/systems described herein utilizing electrode arrays may be configured to maintain the spacing between electrodes of the array and/or electrode geometry across a range of diameters for the expandable member (e.g., about 15 mm to about 45 mm), such that regardless of which value is selected during treatment based on the patient's anatomy, the spacing between the electrodes of the array will be predetermined and known by the user. For similar reasons, other types of treatment members may be configured to provide uniform treatment across a range of diameters of the expandable member in order to treat target tissue consistently across the device. For example, a treatment member comprising a fluid (e.g., a heated fluid, a cryogenic fluid) may uniformly treat (e.g., heat or cool) circumferentially around the tissue. This may allow for the electrode array to provide consistent energy delivery (e.g., pulsed electric field) for tissue lumens of various diameters, thereby ensuring consistent treatment. Furthermore, when transitioning the expandable member to an expanded configuration, a portion comprising a treatment member such as, for example, an electrode array, may expand before one or more pleats unfold.

In some variations, the size and shape of the expandable member may be adjustable based on a configuration of the expandable member. For example, rolling and flattening of an expandable member may be caused by respective retraction and advancement of a sheath (e.g., delivery catheter) over the expandable member. In some variations, the expandable member and the visualization device in the delivery configuration may have a diameter of less than about 17 mm. In some variations, the expandable member in the treatment configuration may have a diameter between about 10 mm and about 60 mm, between about 20 mm and about 50 mm, between about 30 mm and about 50 mm, and between about 40 mm and about 50 mm, including all ranges and sub-values in-between.

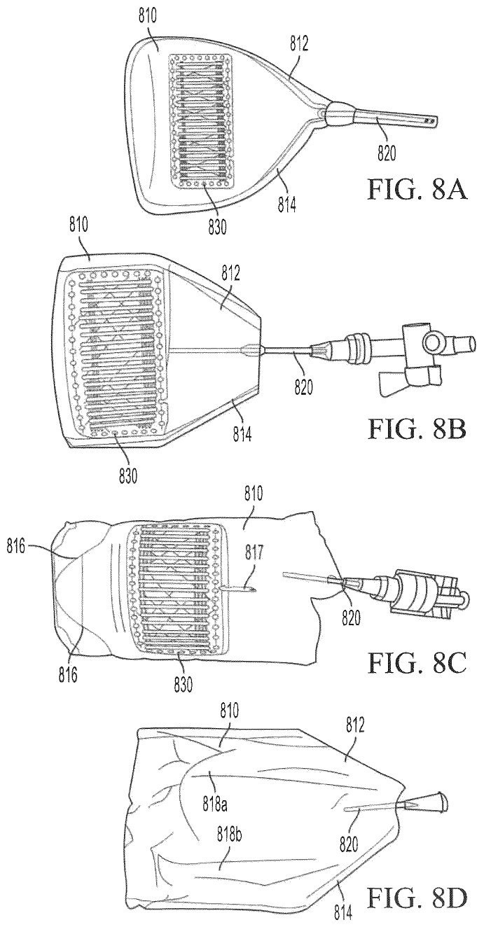

In some variations, the expandable member may comprise one, or more than one, inflatable balloon, wherein each inflatable balloon comprises an electrode array and at least one pleat configured to facilitate flattening the inflatable balloon for placement into a delivery configuration. The pleat may be configured to stretch the target tissue and allow a diameter of the expandable member to be adjustably controlled. For example, as discussed in detail with respect to FIGS. 8C, 10A-10D, 12A-12D, a distal portion of the inflatable balloon may comprise at least one pleat. In an expanded configuration, the pleat may enable the distal end of the inflatable balloon to have a rectangular shape. Additionally or alternatively, one or more lateral portions of the inflatable balloon may comprises one or more longitudinal pleats. The longitudinal pleats may allow a diameter of the inflatable balloon to be adjustable. For example, when a longitudinal pleat is in an unfolded configuration and the inflatable balloon is in the expanded configuration, the diameter of the inflatable balloon may be increased relative to the pleat in a folded configuration. In some variations, an electrode array may not be disposed on the pleat such that when in the unfolded and expanded configuration, at least the portion of the expandable member corresponding to the pleat may directly contact tissue. Accordingly, an electrode array may not contact tissue about the entire circumference of the expandable member.

The pleat may be configured to transition the expandable member (e.g., an inflatable balloon) between a folded configuration having a first diameter and an unfolded configuration having a second diameter larger than the first diameter. The second diameter may be between about 1 mm and 10 mm larger than the first diameter. For example, the expandable member may be partially inflated in a folded treatment configuration where the pleat remains folded but in contact with tissue. The folded configuration may be appropriate for relatively smaller tissue lumens. The expandable member may be further inflated in an unfolded treatment configuration where the pleat is unfolded such that the expandable member may have a larger diameter relative to the folded configuration. The unfolded configuration may be appropriate for relatively larger tissue lumens.

In some variations, the expandable member may be asymmetric relative to a longitudinal axis of the expandable member to facilitate a transition between a delivery configuration and treatment configuration of the expandable member. For example, the asymmetry of the expandable member may facilitate rolling the expandable member at least partially around the visualization device when transitioning from the treatment configuration to the delivery configuration. In some variations, a length of a taper and/or an angle of the taper relative to a longitudinal axis may be different between a first taper (e.g., left side taper) and a second taper (e.g., right side taper). A n asymmetric taper may allow one of the tapers to preferentially roll beneath the other taper to promote the transition of the expandable member into the delivery configuration. For example, a larger taper may encounter less resistance in rolling around a visualization device. Therefore, having asymmetrical tapers may bias different portions of the expandable member when withdrawing an expandable member into a sheath (e.g., transitioning from a treatment configuration to a delivery configuration).

In some variations, the expandable member may be asymmetrically coupled to the elongate body in order to facilitate a transition of the expandable member into a delivery configuration. For example, the expandable member may be eccentrically coupled to the elongate body such that a longitudinal axis of the elongate body does not align with the longitudinal axis of the expandable member. In this manner, a first lateral portion of the expandable member may have a smaller surface area than a second lateral portion of the expandable member. In some variations, the elongate body may be coupled to a sidewall of the expandable member to facilitate a transition of the expandable member into a delivery configuration. For example, the elongate body may be coupled to the sidewall of the expandable member such that when transitioned to the delivery configuration, a first lateral portion of the expandable member may overlap a second lateral portion of the expandable member when placed around a visualization device.

In some variations, one or more edges of the expandable member and/or electrode array may be atraumatic to reduce tissue damage. For example, one or more edges of the expandable member may comprise a seam formed via one or more of a thermal seal and an adhesive. In some variations, one or more edges of the electrode array may be coupled to the expandable member via one or more of a thermal seal and an adhesive.

In some variations, the expandable member may have a width of at least 10 mm, between about 10 mm and about 100 mm, between about 10 mm and about 50 mm, between about 10 mm and about 30 mm, between about 20 mm and about 40 mm, between about 30 mm and about 100 mm, between about 30 mm and about 80 mm, between about 30 mm and about 60 mm, between about 30 mm and about 50 mm, including all ranges and sub-values in-between. In some variations, the expandable member may comprise a length of between about 10 mm and about 300 mm, between about 10 mm and about 200 mm, between about 10 mm and about 100 mm, between about 50 mm and about 300 mm, between about 50 mm and about 200 mm, between about 100 mm and about 300 mm, between about 100 mm and about 200 mm, including all ranges and sub-values in-between. In some variations, the expandable member may comprise a wall thickness of between about 0.02 mm and about 0.5 mm, between about 0.02 mm and about 0.3 mm, between about 0.02 mm and about 0.3 mm, between about 0.02 mm and about 0.2 mm, between about 0.1 mm and about 0.5 mm, between about 0.1 mm and about 0.3 mm, between about 0.2 mm and about 0.5 mm, between about 0.3 mm and about 0.5 mm, including all ranges and sub-values in-between.

In some variations, the expandable member may comprise one or more sensors configured to determine a configuration of the expandable member. For example, the sensor may comprise one or more inductive coils configured to measure proximity between the expandable member and the sheath such that a position of the expandable member may be determined. For example, one or more sensors may be disposed along a length of the expandable member. In some variations, a configuration of the expandable member may be confirmed via visual confirmation and via the sensor data.

Additionally or alternatively, the tissue treatment device may comprise a plurality of expandable members (e.g., inflatable member, inflatable balloon, support, basket, frame, cage) disposed in a serial configuration. For example, the tissue treatment device may comprise between about one and about ten expandable members, between about three and about ten expandable members, between about five and about ten expandable members, between about one and about five expandable members, between about one and about eight expandable members, between about three and about six expandable members, including all ranges and sub-values in-between. In some variations, the expandable member may comprise a width of between about 1.5 inches and about 4 inches, between about 2 inches and about 3 inches, between about 2 inches and 4 inches, between about 1.5 inches and about 3 inches, including all ranges and sub-values in-between.

### EXAMPLES

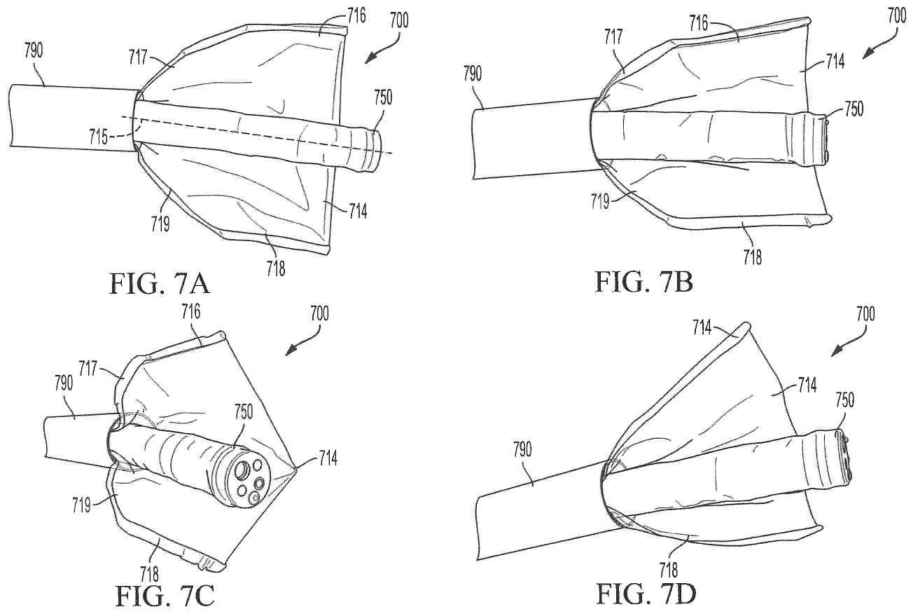

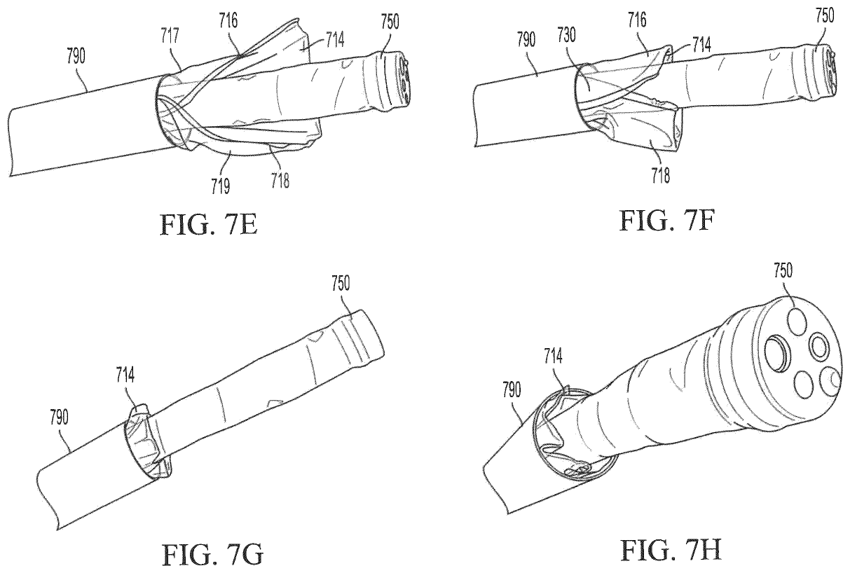

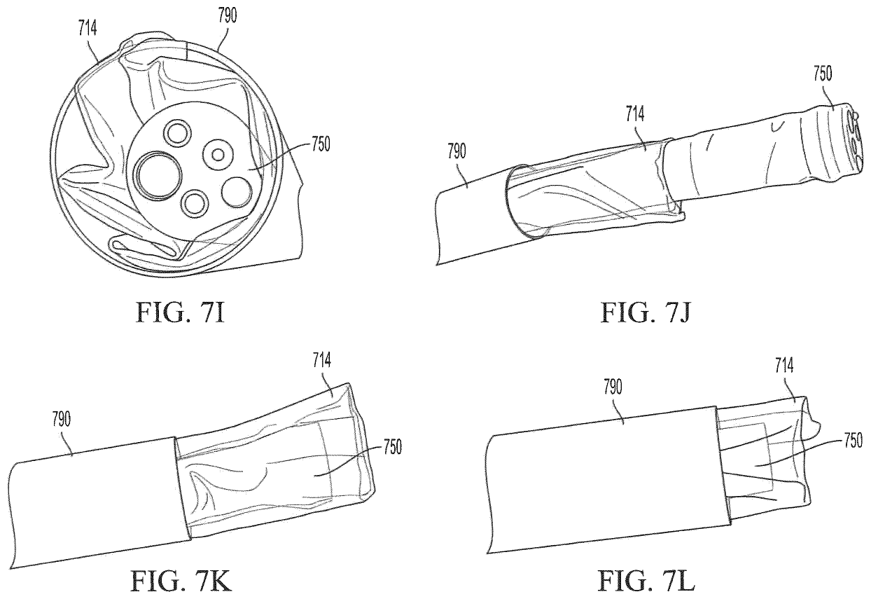

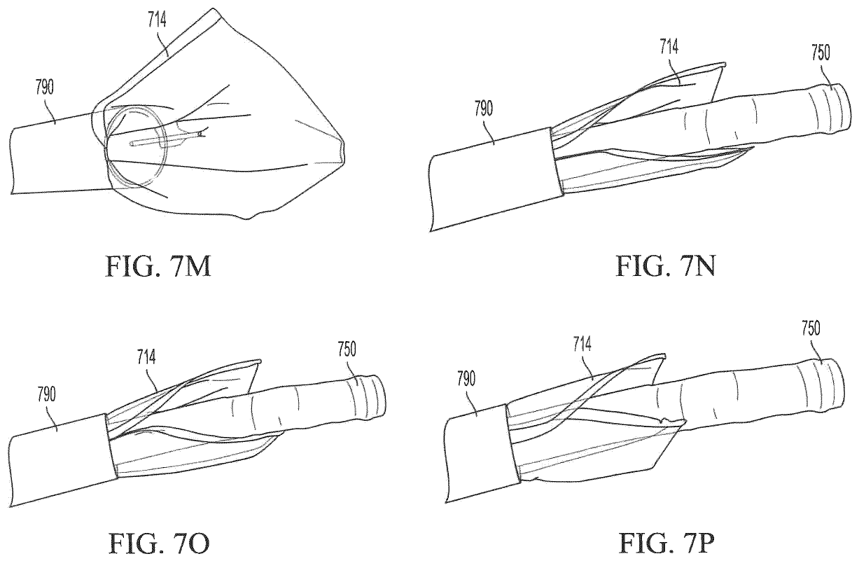

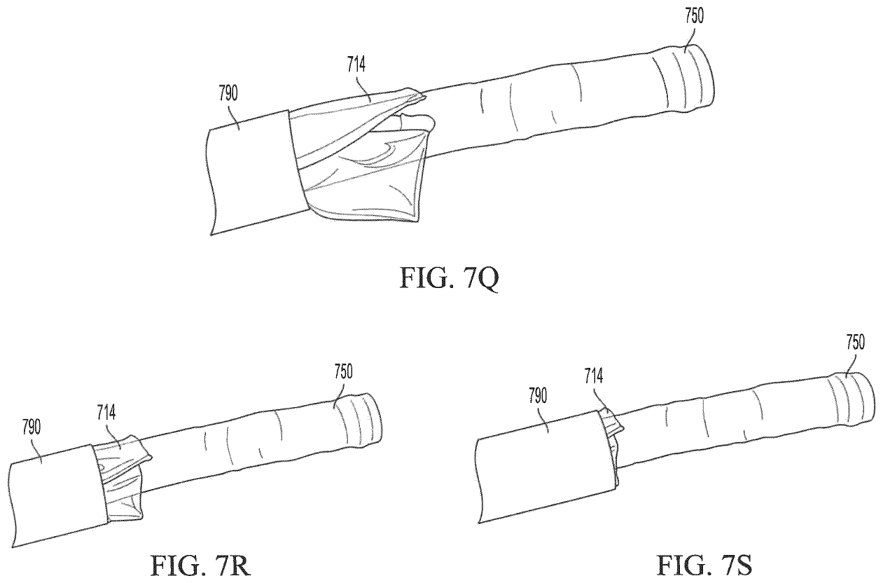

FIGS. 7A-7G are images of side views of illustrative variations of a tissue treatment system (700) including a sheath (790) receiving an expandable member (714) (e.g., inflatable balloon) and visualization device (750) (e.g., endoscope). In FIGS. 7A-7F, the sheath (790) is withdrawn relative to the expandable member (714) and the visualization device (750) in order to depict the spatial relationship between the expandable member (714) and visualization device (750). The expandable member (714) may generally be aligned with a distal portion (e.g., distal end) of the visualization device (750) in a delivery configuration. FIG. 7A shows the expandable member (714) substantially unconstrained by the sheath (790) such that the distal portion of the expandable member (714) biases (e.g., naturally returns) to a flattened configuration. However, as the sheath (790) is progressively advanced over the expandable member (714) and visualization device (750) (see FIGS. 7B-7H) from a proximal end of the expandable member (714), a proximal portion of the expandable member (714) begins to couple concentrically (e.g., wrap, surround, encircle) about the visualization device (750). That is, the expandable member (714) is disposed between an inner surface of the sheath (790) and an outer surface of the visualization device (750).

In some variations, the expandable member (714) may define a longitudinal axis (715) parallel to a longitudinal axis of the visualization device (750) and sheath (790). In some variations, the expandable member (714) may comprise a first portion (716) on a first side of the longitudinal axis and a second portion (718) on a second side of the longitudinal axis opposite the first side. For example, the first portion (716) may be a first lateral portion (e.g., left portion) and the second portion (718) may be a second lateral portion (e.g., right portion). The first lateral portion (716) and the second lateral portion (718) may have the same area or different area. In some variations, the first portion (716) may comprise a first proximal taper (717) and the second portion (718) may comprise a second proximal taper (719). As described in more detail herein, the first proximal taper (717) and the second proximal taper (719) may be symmetric or asymmetric. As the expandable member (714) is withdrawn into the sheath (790), the first proximal taper (717) and the second proximal taper (719) provide an edge that interfaces smoothly with a distal edge of the sheath (790) to aid the transition of the expandable member (714) from a flattened configuration having a generally planar shape to a delivery configuration having a generally concentric shape. In some variations, the proximal tapers (717, 719) and the longitudinal axis (715) form an angle between about 10 degrees and about 80 degrees, between about 30 degrees and about 60 degrees, including all ranges and sub-values in-between.

As the proximal portion of the expandable member (714) is drawn into the sheath (790), the expandable member (714) is constrained between the sheath (790) and the visualization device (750) and positioned circumferentially about the visualization device (750). For example, the first portion (716) is positioned circumferentially about the visualization device (750) in a first direction (e.g., counter-clockwise direction when viewed from a distal end of the visualization device) and the second portion (718) is positioned circumferentially about the visualization device (750) in a second direction opposite the first direction (e.g., clockwise direction). Furthermore, as the expandable member (714) is withdrawn into the sheath (790), the expandable member (714) may transition from the substantially flattened configuration shown in FIG. 7A to the generally concentric delivery configuration. For example, FIGS. 7C-7F show the expandable member (714) wrapping itself around the visualization device (750) where the ends of the first lateral portion (716) and second lateral portion (718) gradually meet and overlap. FIG. 7F shows an overlap (730) of the first lateral portion (716) and the second lateral portion (718) that increases as the expandable member (714) is further withdrawn into the sheath (790). In the delivery configuration, the first lateral portion (716) may overlap the second lateral portion (718) along a length of the expandable member (714). FIGS. 7J-7L shows the expandable member (714) circumferentially disposed around a circumference of the visualization device (750). When housed in the sheath (790), the first lateral portion (716) is rolled around the visualization device (750) and the second lateral portion (718) is rolled around the visualization device (750) and partially overlaps the first lateral portion (716). For example, the first lateral portion (716) is rolled under the second lateral portion (718). FIGS. 7K and 7L show the expandable member (714) in the delivery configuration advanced slightly distal to the visualization device (750). FIGS. 7G and 7H show the expandable member (714) nearly completely withdrawn into the sheath (790). FIG. 7I is a front end view of the sheath (790) receiving a distal end of the expandable member (714) and the visualization device (750). In some variations, the expandable member (714) may comprise one or more folds or pleats when disposed circumferentially (e.g., rolled, wrapped) about the visualization device (750) within the sheath (790).

FIGS. 7M-7S depict an example of the transition of an expandable member (714) from a treatment configuration into a delivery configuration. In the perspective view of FIG. 7M, the expandable member (714) is in an uninflated configuration where a proximal portion of the expandable member (714) is in the process of being withdrawn into a distal end of the sheath (790). The side views of FIGS. 7N-7S show the expandable member (714) being further withdrawn into the sheath (790) where the proximal taper facilitates the transition of the expandable member (714) from a flattened configuration to a circumferential shape of the delivery configuration. In FIGS. 7N-7S, the visualization device (750) is shown distal to the sheath (790) and expandable member (714) for the sake of illustration. However, the visualization device (750) may be disposed within a distal end of the sheath (790) during when the expandable member (714) transitions from the treatment configuration to the delivery configuration and vice versa. In the delivery configuration, the tissue treatment device may be translated through one or more body lumens or to another portion of tissue to be treated within the same portion of anatomy.

FIGS. 8A-8D are images of a side view of illustrative variations of an expandable member (810) coupled to an electrode array (830) and an inflation lumen (820). In some variations, the expandable member (810) may comprise one or more of a first proximal taper (812), a second proximal taper (814), a distal pleat (816), and a longitudinal pleat (818). In some variations, the electrode array (830) may be coupled to the expandable member (810). The electrode array (830) may be generally perpendicular to a longitudinal axis of the expandable member (810). In some variations, the electrode array (830) may extend at least partially (e.g., but not completely) laterally across the expandable member (810). The expandable members (810) shown in FIGS. 8A, 8B, and 8D are shown in an uninflated, flattened configuration. In some variations, as shown in FIG. 8C, the electrode array (830) may be coupled to a suction lumen (817) configured to apply suction to the electrode array (830) and tissue. In FIG. 8C, the distal pleat (816) may comprise one or more folds configured to facilitate flattening the inflatable balloon for placement into a delivery configuration and expansion in a treatment configuration. In some variations, a distal portion of the expandable member (810) may comprise a square shape to reduce a curvature (e.g., doming) of the electrode array (830) to facilitate the generally planar shape of the flattened configuration.

In some variations, the longitudinal pleats (818a, 818b), as shown in FIG. 8D, may comprise one or more folds configured to increase a circumference of the expandable member in an inflated treatment configuration. For example, a first lateral portion may comprise a first longitudinal pleat (818a) and a second lateral portion (818b) may comprise a second longitudinal pleat. The pleat in a folded configuration may minimize a height of the expandable member while the pleat in an unfolded configuration may increase a height of the expandable member. In some variations, an electrode array is not disposed within the longitudinal pleat such that a first electrode array on a first side of the expandable member may be spaced apart from a second electrode array on a second side of the expandable member by about the height of the unfolded pleat(s). FIG. 8D shows the first proximal taper (812) symmetric with respect to the second proximal taper (814). As discussed herein, the proximal tapers (812, 814) may be configured to facilitate transition of the expandable member (810) between the delivery configuration and the treatment configuration. Alternatively, the first proximal taper (812) and second proximal taper (814) may have different lengths and angles with respect to a longitudinal axis of the expandable member (810).

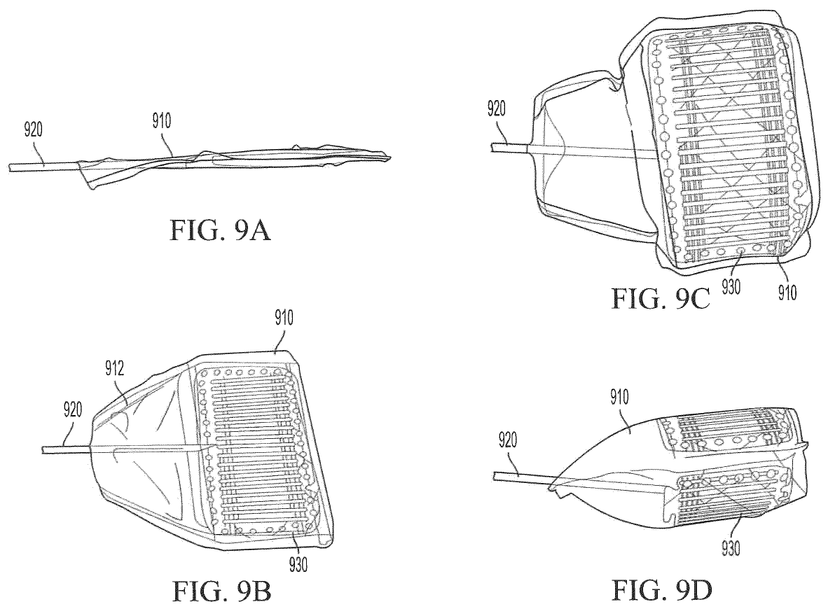

FIGS. 9A-9D are illustrations of side views of an illustrative variation of an expandable member (910) in respective flattened and inflated configurations. The expandable member (910) may be coupled to an inflation lumen (920) configured to inflate the expandable member (910) with a fluid (e.g., gas, liquid). The expandable member (910) is shown in a flattened configuration FIGS. 9A and 9B having a generally planar shape and flexibility to wrap around a visualization device when disposed within a sheath. The expandable member (910) may be coupled to an electrode array (930) configured to generate a pulsed electric field to treat tissue. The expandable member (910) and electrode array (930) may bias towards the flattened configuration when unconstrained. The expandable member (910) is shown in an expanded configuration in FIGS. 9C and 9D configured to contact tissue in a body lumen. Any of the expandable members described herein in the treatment configuration may have a predetermined flexibility configured to conform to a shape of the tissue to which it is engaged.

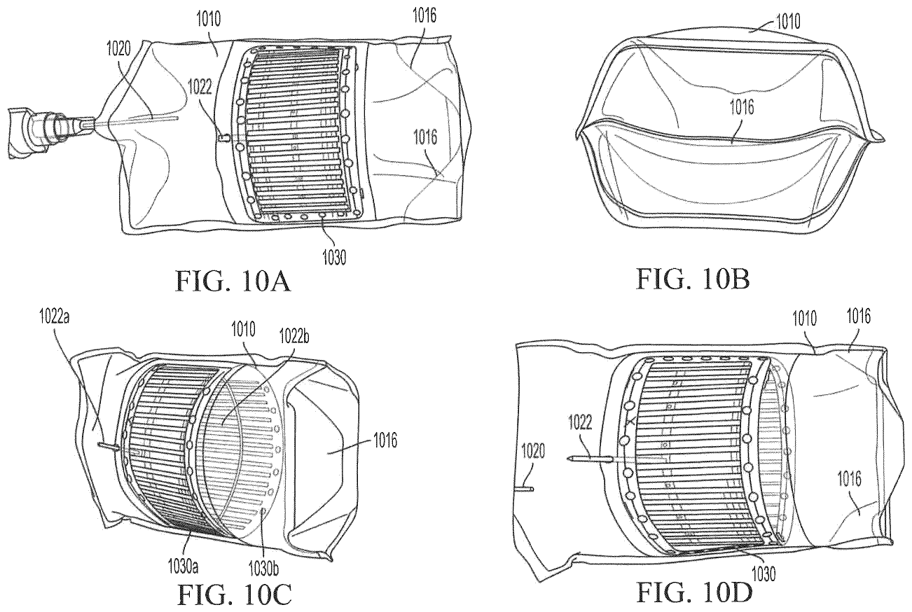

FIGS. 10A-10D illustrate side, front, and perspective views of an illustrative variation of an expandable member (1010) in an expanded configuration. The expandable member (1010) may be coupled to an inflation lumen (1020) configured to inflate the expandable member (1010) with a fluid (e.g., gas, liquid). The expandable member (1010) may be coupled to an electrode array (1030) configured to generate a pulsed electric field to treat tissue. The electrode array (1030) may be coupled to one or more suction lumens (1022) configured to suction tissue to the electrode array (1030). For example, FIG. 10C depicts a first electrode array (1030a) coupled to a first suction lumen (1022a) and a second electrode array (1030b) coupled to a second suction lumen (1022b).

In some variations, the expandable member (1010) may comprise a distal pleat (1016) configured to unfold in the expanded configuration. From the top view perspective of FIG. 10A, the expandable member (1010) may have a distal portion having a generally rectangular shape. From the front view perspective of FIG. 10B, the distal end of the expandable member (1010) may have a generally rectangular or hexagonal shape. In the expanded configuration, the electrode array (1030) may have a generally curved (e.g., elliptical, oval, teardrop, matching a curvature of a tissue lumen) shape. As shown in FIG. 10B, the distal pleat (1016) may extend across a width of the expandable member (1010).

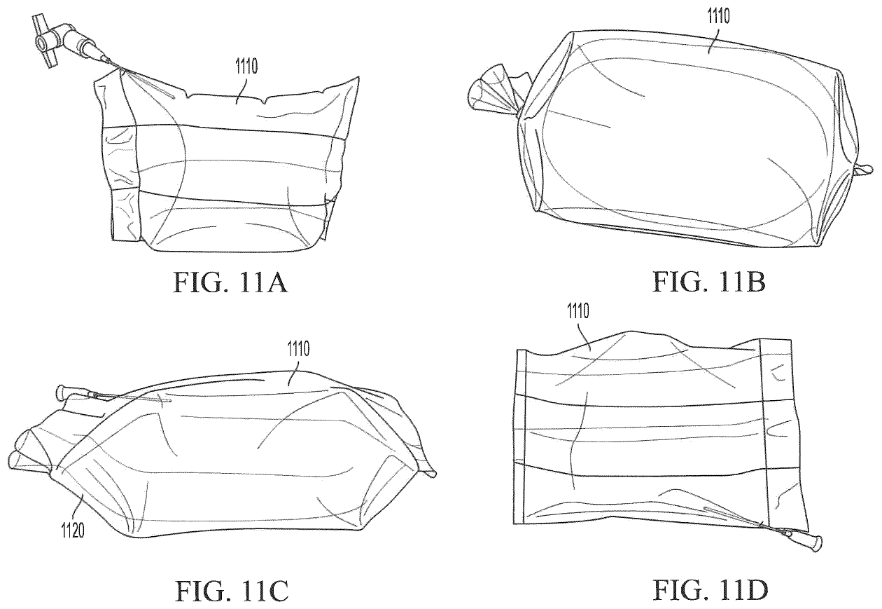

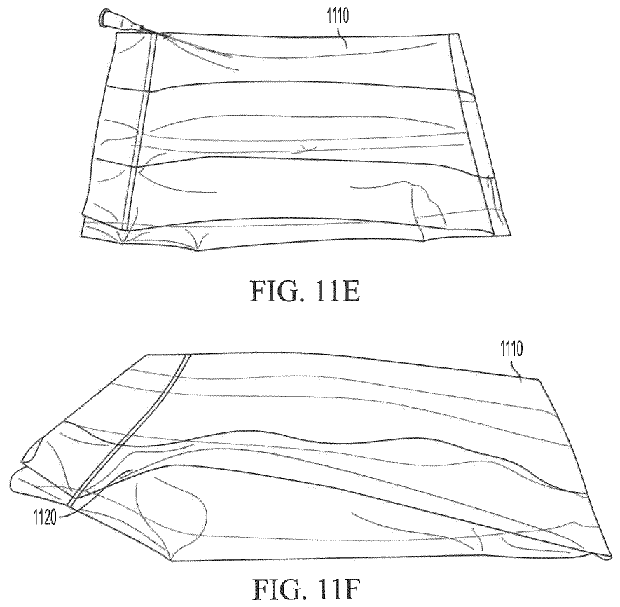

FIGS. 11A-11F illustrate side views of an illustrative variation of an expandable member (1110) comprising a pleat (1120) in an expanded configuration. As shown in FIGS. 11B and 11C, the pleat (1120) may be unfolded in the expanded configuration. In some variations, the expandable member (1110) may have a generally rectangular or hexagonal shape from a front view of the expandable member (1110). FIGS. 11E and 11F are images of side and perspective views of an illustrative variation of an expandable member (1110) in an unexpanded configuration where the pleat (1120) may be folded to promote the generally planar shape of the expandable member (1110).

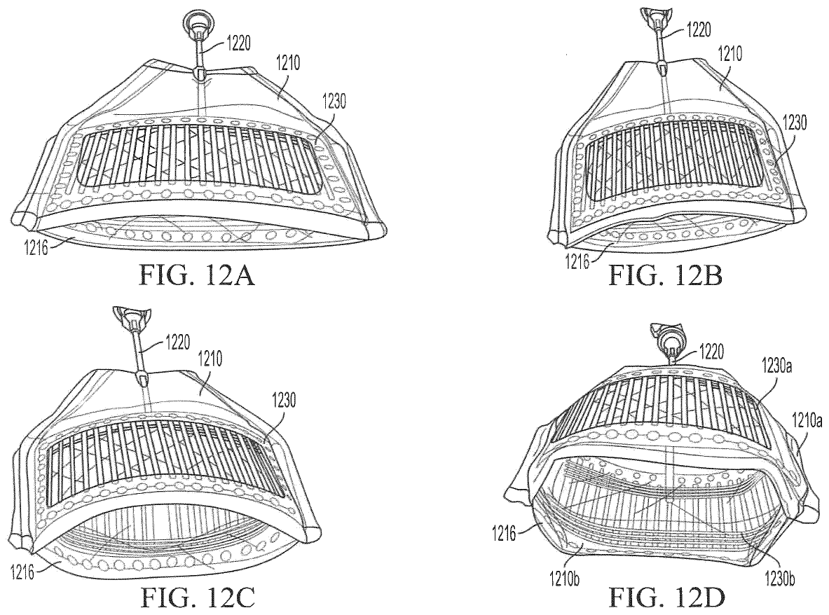

FIGS. 12A-12D illustrate end views of an illustrative variation of an expandable member (1210) transitioning between an unexpanded configuration and an expanded configuration. The expandable member (1210) may be coupled to an inflation lumen (1220) configured to inflate the expandable member (1210) with a fluid (e.g., gas, liquid). The expandable member (1210) is shown in a flattened configuration FIGS. 12A and 12B having a generally planar shape and flexibility to wrap around a visualization device when disposed within a sheath. The expandable member (1210) may be coupled to one or more electrode arrays (1230) configured to generate a pulsed electric field to treat tissue. For example, FIG. 12D shows a first side (1210a) of the expandable member (1210) coupled to a first electrode array (1230a) and a second side (1210b) of the expandable member (1210) coupled to a second electrode array (1230b). The expandable member (1210) and electrode array (1230) may bias towards the flattened configuration when unconstrained. The expandable member (1210) is shown in an expanded configuration in FIGS. 12C and 12D configured to contact tissue in a body lumen. In the expanded configuration, the electrode array (1230) may have a generally curved (e.g., elliptical, oval) shape.

In some variations, the expandable member (1210) may comprise a distal pleat (1216) configured to unfold in the expanded configuration. From the front perspective view of FIG. 12D, the expandable member (1210) may have a distal portion having a generally rectangular or hexagonal shape. In the expanded configuration, the electrode array (1230) may have a generally curved (e.g., elliptical, oval) shape. As shown in FIGS. 12C and 12D, the distal pleat (1216) may extend across a width of the expandable member (1210). As shown in FIG. 12D, the distal pleat (1216) may extend across a width of the expandable member (1210).

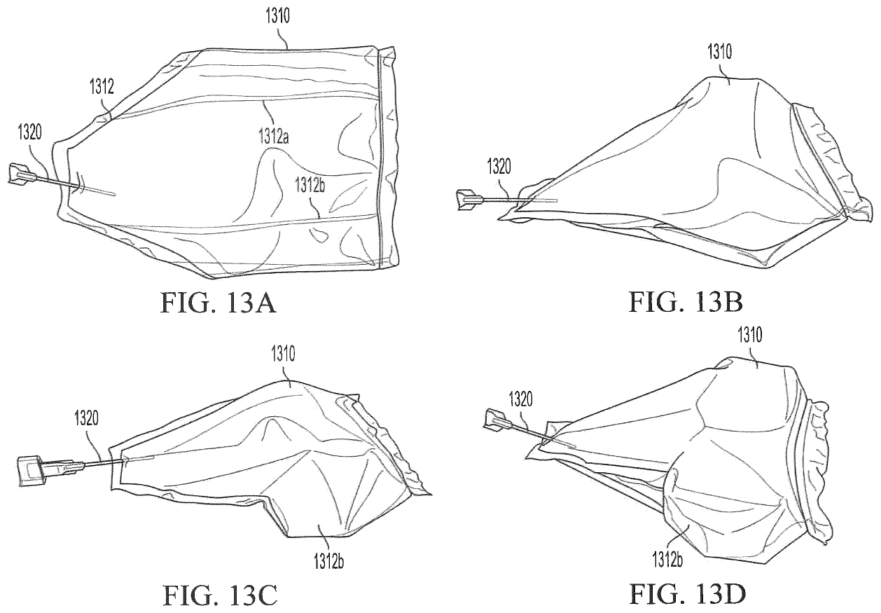

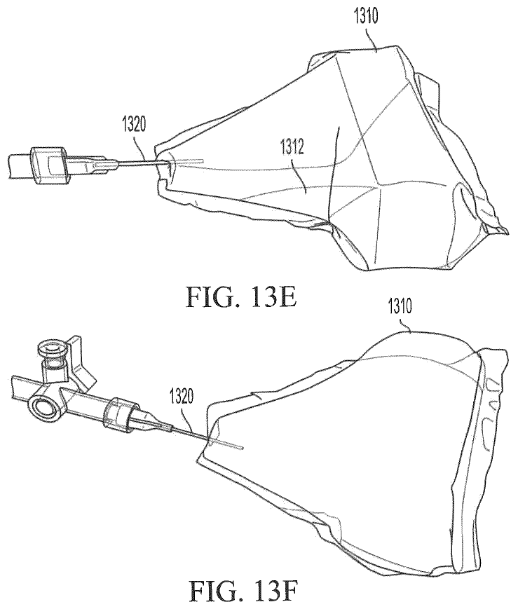

FIGS. 13A-13F illustrate side views of an illustrative variation of an expandable member (1310) transitioning between an unexpanded configuration and an expanded configuration. The expandable member (1310) may be coupled to an inflation lumen (1320) configured to inflate the expandable member (1310) with a fluid (e.g., gas, liquid). The expandable member (1310) may be coupled to an electrode array (not shown) configured to generate a pulsed electric field to treat tissue. In some variations, the expandable member (1310) may comprise one or more longitudinal pleats (1312a, 1312b) configured to unfold in the expanded configuration. The longitudinal pleats (1312a, 1312b) may be configured to increase a circumference of the expandable member in an inflated treatment configuration. From the top view perspective of FIG. 13A, the expandable member (1310) may have a first longitudinal pleat (1312a) on a first lateral portion of the expandable member (1310) and a second longitudinal pleat (1312b) on a second lateral portion of the expandable member (1310). The longitudinal pleats (1312a, 1312b) may extend from a distal end to a proximal taper (1312) of the expandable member (1310). From the top view of FIG. 13A, the distal end of the expandable member (1310) may have a generally rectangular, circular, elliptical, or oval shape. From a side perspective, a height of the expandable member (1310) may generally increase in a distal direction.

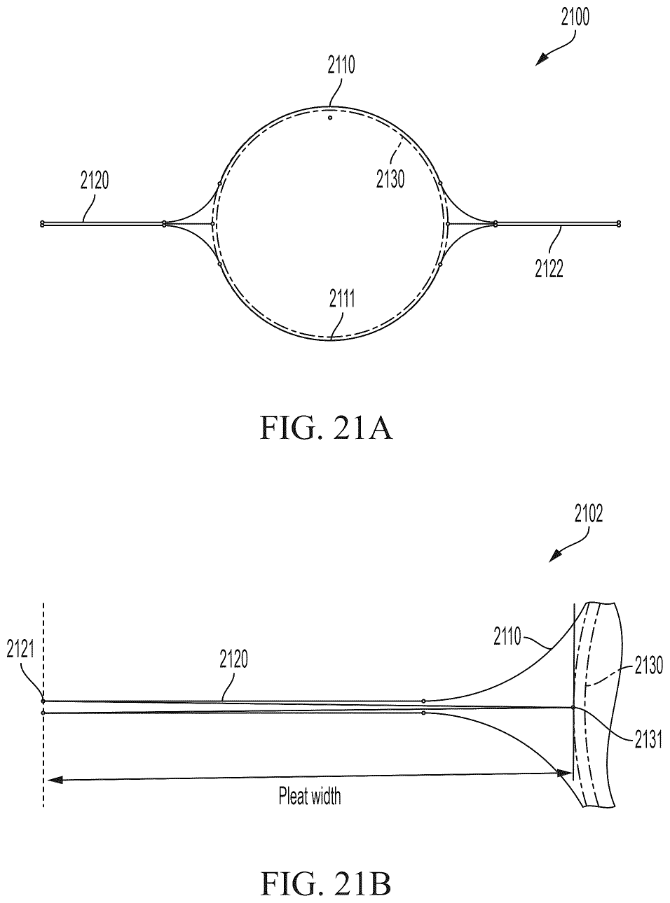

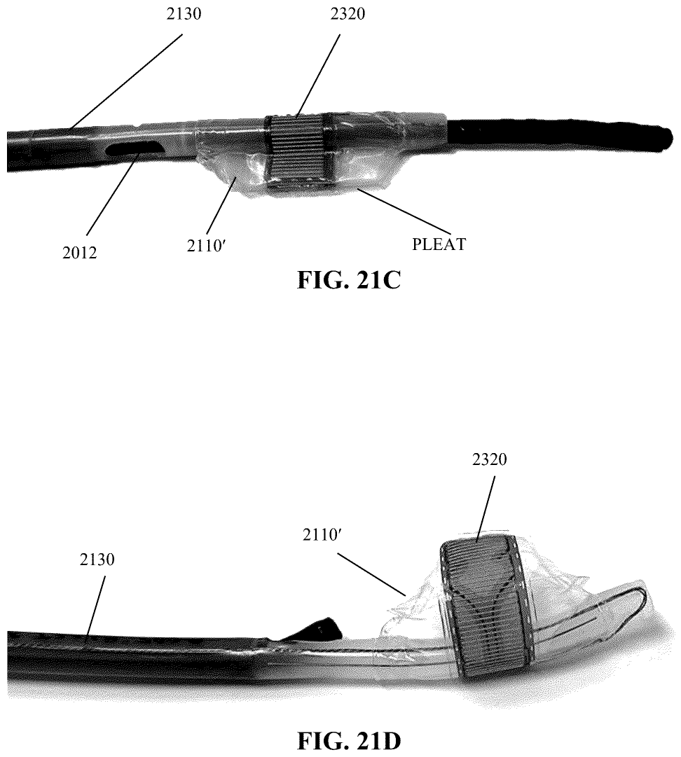

FIGS. 21A and 21B are schematic cross-sectional diagrams (2100, 2102) of an illustrative variation of pleats (2120, 2122) of an expandable member (2110) and an overtube (2130). For example, the expandable member (2110) may comprise a first pleat (2120) and a second pleat (2122) disposed opposite the first pleat (2120). A width of the first pleat (2120) may be measured from an outermost edge (2121) of the first pleat (2120) to an outer diameter (2131) of the overtube (2130). A width of the pleat in the delivery configuration may be about 0.1 mm to about half of a difference between a width of the expandable member in the delivery configuration and a diameter (e.g., maximum dimension) of the elongate body. As shown in FIG. 21B, the pleat may form a W-shape, but may also form a V-shape.

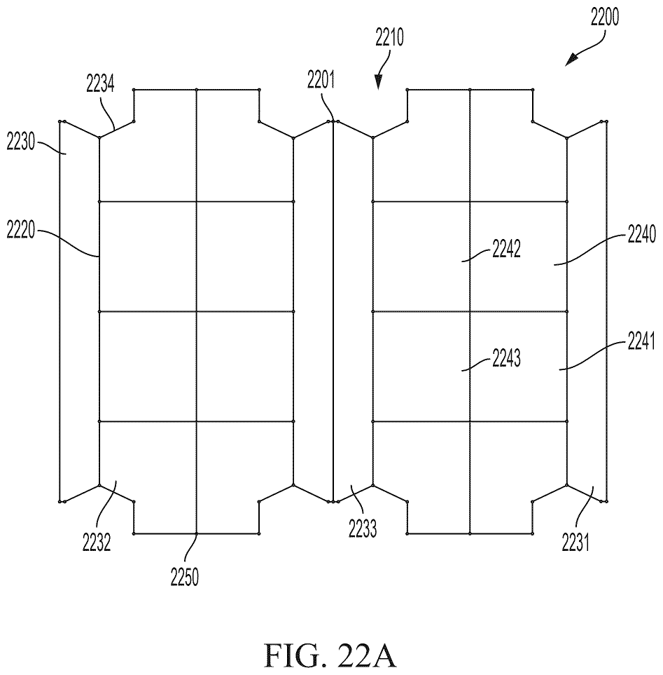

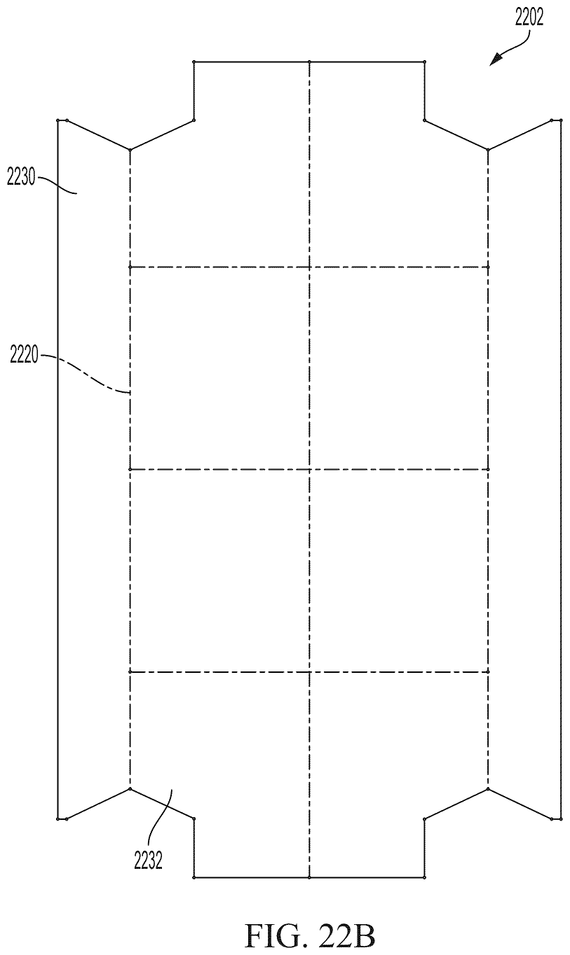

FIGS. 22A and 22B are schematic diagrams (2200, 2202) of an illustrative variation of an unassembled (e.g., flat) expandable member (2210). Shown there is a pattern (e.g., a laser cut pattern) of an expandable member (2200, 2202) in the form of a balloon where the centerline (2201) denotes the two halves of the balloon. The lateral edges of the two halves may be brought together and sealed to form the balloon having a circumference. The outermost edge (2121) of the first pleat (2120) in FIG. 21B corresponds to pleat edge (2220) of the expandable member (2200, 2202) in FIGS. 22A and 22B. A midpoint (2111) of the expandable member (2110) in FIG. 21A corresponds to a midpoint (2250) of the expandable member (2210) in FIG. 22A. With respect to FIG. 22B, the pleat may comprise an inner pleat (2230) (e.g., portion of the pleat configured to fold inward) and an outer pleat (2232) (e.g., portion of the pleat configured to face outward/away from an overtube). With respect to FIG. 22A, a treatment member such as an electrode array (e.g., flex circuit, substrate) may be placed between the first pleat (2231) and a second pleat (2233) over the four quadrants (2240-2243).

As discussed herein, the expandable member 2110 may be asymmetric relative to the overtube 2130. FIGS. 21C and 21D illustrate one embodiment or variation of an asymmetric expandable member. FIG. 21C illustrates an asymmetric expandable member 2110′ wherein a portion of the asymmetric expandable member 2110′ is secured around or wrapped around or surrounds a portion of the overtube 2130. The asymmetric expandable member 2110′ comprises an electrode array 2320 surrounding, or partially surrounding, the asymmetric expandable member 2110′. In some variations, the electrode array 2320 may also surround, or partially surround, the portion of the overtube 2130 to which the expandable member 2110′ is secured, wrapped or surrounding. The expandable member 2110′ of FIG. 21C is shown in a deflated configuration and before wrapping the expandable member 2110′ around the overtube 2130. As discussed herein, the expandable member 2110′ may comprise a pleat.

FIG. 21D illustrates the asymmetric expandable member 2110′ of FIG. 21C in an expanded or inflated configuration.

In some variations, the relative dimensions between the pleat, electrode array, bonding layer, and the like may facilitate consistent electrode geometry across different diameters of the expandable member. In some variations, a pleat and electrode array may be separated (e.g., offset) by a predetermined distance (such as, for example, at least about 0.5 inches) configured to reduce puckering and provide a portion for the ballon to taper between the pleat and electrode array.

In some variations, a bonding layer may be used to attach a treatment member, such as an electrode array, to an expandable member such that the expandable member may expand while maintaining the geometry of the treatment member. For example, a bonding layer disposed over the electrode array and the expandable member may extend about 0.02 inch to about 0.125 inch beyond the dimensions (e.g., length, width) of the electrode array to facilitate bonding of the electrode array to the expandable member. The bonding layer may cover a perimeter of the electrode array and/or a portion of the electrode array (e.g., not covering the electrodes). The bonding layer may comprise one or more of a thermoplastic urethane, thermoplastic elastomer, polyethylene terephthalate, polyimide, nylon, and biaxially-oriented polyethylene terephthalate

In some variations, the pleat may form a taper to facilitate atraumatic advancement of the expandable member through the patient. For example, the pleat may taper and form an angle with respect to a longitudinal axis of the expandable member of about 0 degrees to about 60 degrees, about 1 degree to about 60 degrees, about 10 degrees to about 50 degrees, about 20 degrees to about 40 degrees, about 30 degrees to about 60 degrees, about 1 degree to about 30 degrees, about 1 degree to about 45 degrees, about 15 degrees to about 60 degrees, about 10 degrees, about 20 degrees, about 25 degrees, about 30 degrees, about 40 degrees, about 50 degrees, about 60 degrees, including all ranges and sub-values therebetween. In some variations, a width of each pleat of the plurality of pleats is up to half of a difference between a width of the electrode array and a diameter of the elongate body (e.g., half of 1.58 inches minus 0.98 inches). In some variations, a dimension (e.g., length, width) of the electrode array may be about 1 inch to about 3 inch, about 1 inch to about 2.5 inch, about 1 inch to about 2 inch, about 1.5 inch to about 3 inch, about 2 inch to about 3 inch, about 1 inch, about 1.25 inch, about 1.5 inch, about 1.75 inch, about 2 inch, about 2.25 inch, about 2.5 inch, about 2.75 inch, about 3 inch, including all ranges and sub-values therebetween.

In some variations, the expandable member may comprise portions having different rigidities (e.g., durometer) in order to promote a predetermined expansion sequence when transitioning from an unexpanded configuration to an expanded configuration. In some variations, the expandable member may comprise a first layer having a first durometer and a second layer having a second durometer different than the first durometer. The second layer may be disposed over one or more predetermined portions of the expandable member. The second durometer may be higher than the first durometer such that the second layer disposed over the first layer adds flexural rigidity (e.g., reduces compliance) where desired. For example, portions of the expandable member coupled to the electrode array may include the first layer and the second layer while portions of the expandable member not coupled to the expandable member (e.g., pleats) may include just the first layer and vice versa. In this manner, different portions of the expandable member may expand at different rates when the expandable member is inflated.

### Treatment Member/Electrode Array

Generally, treatment members described herein may be configured to treat tissue using any of the treatment modalities described herein, including but not limited to thermal energy (e.g., heat-based ablation, cryogenic fluid), pulsed-electric field energy, ultrasonic energy (e.g., piezoelectric transducer), vapor energy, radiofrequency energy, laser energy, mechanical energy (e.g., blade), and the like. For example, the treatment member may comprise one or more of an electrode, an electrode array, a piezoelectric transducer, a laser, a blade, and a thermal element.

Further, as noted above, in some variations, the treatment members described herein may comprise one or more electrodes and/or electrode arrays configured to treat tissue of a patient. For example, in some variations, the electrode array may engage the tissue and be energized to treat a predetermined portion of tissue to resurface the or otherwise treat the tissue. For example, tissue may undergo cell lysis using PEF energy during a treatment procedure. PEF energy tissue treatment may be uniformly delivered at a predetermined depth (e.g., about 1 mm) to quickly and precisely treat any part of the GI tract, including the duodenum and esophagus without significant damage to surrounding (e.g., deeper) tissue.

In some variations, the electrode array may comprise a flexible circuit substrate, wherein the flexible circuit substrate comprises one or more of the group consisting of: all-Polyimide laminate, Polyester (PET), Polyethylene Naphthalate (PEN), Polyamide, Liquid Crystal Polymer (LCP), and PTFE.

In some variations, tissue treatment characteristics may be controlled by the size, shape, spacing, composition, and/or geometry of the electrode array. For example, the electrode array may be flexible to conform to non-planar tissue surfaces. In some variations, the electrode array may be embossed or reflowed to form a non-planar electrode surface. In some variations, the electrode array may comprise a tissue contact layer. The tissue contact layer may function as a salt bridge between the electrodes and tissue. In some variations, the electrode array may comprise a hydrophilic coating. Additionally or alternatively, the electrode array may be electrically divided into sub-arrays to reduce drive current requirements. In some variations, the sub-arrays may correspond to the plurality of sections described herein.

In some variations, raised and/or rounded (e.g., semi-ellipsoid) electrodes may generally promote more reliable contact with tissue than flat electrodes and therefore a more uniform electrical field and improved treatment outcomes. For example, tissue contact (e.g., apposition) with the electrodes completes an electrical circuit during energy delivery and therefore provides the resistance in the circuit for a uniform electric field distribution. The raised and/or rounded (e.g., semi-ellipsoid) electrodes may reduce sharp edges to reduce arcing. The spaced-apart electrodes of the electrode array may further reduce ion concentration and associated electrolysis. The electrode array configurations (e.g., geometry, spacing, shape, size) shown and described herein provide uniform and spaced-apart electrodes that also allow a corresponding expandable member to repeatedly expand and compress. For example, a predetermined spacing between electrodes may be maintained as an expandable member upon which the electrode array is disposed increases and decreases in diameter.

In some variations, one or more of the electrodes (e.g., a plurality of the electrodes, a portion of the electrodes in an array, all of the electrodes in an array) may comprise one or more biocompatible metals such as gold, titanium, stainless steel, nitinol, palladium, silver, platinum, combinations thereof, and the like. In some variations, one or more electrodes (e.g., a plurality of the electrodes, a portion of the electrodes in an array, all of the electrodes in an array) may comprise an atraumatic (e.g., blunt, rounded) shape such that the electrode does not puncture tissue when pressed against tissue. For example, the electrode array may engage an inner circumference of the duodenum.

In some variations, the electrode array may be connected by one or more leads (e.g., conductive wire, lead wire) to a signal generator. For example, a lead may extend through an elongate body (e.g., outer catheter, outer elongate body) to the electrode array. One or more portions of the lead may be insulated (e.g., PTFE, ePTFE, PET, polyolefin, parylene, FEP, silicone, nylon, PEEK, polyimide). The lead may be configured to sustain a predetermined voltage potential without dielectric breakdown of its corresponding insulation. In some variations, the electrode array may be coupled to the expandable member via a thermal seal.

In some variations, an electrode array may comprise a plurality of elongate electrodes in a substantially parallel or interdigitated configuration. The shape and configuration of the electrode arrays described herein may generate an electric field of predetermined strength (e.g., between about 400 V/cm and about 7,500 V/cm) at a predetermined tissue depth (e.g., about 0.7 mm, about 1 mm) without excess heat, breakdown, steam generation, and the like. By contrast, some electrode configurations comprise a geometry (e.g., radius of curvature) where the electric fields generated decreases too quickly without application of very high voltages (e.g., thousands of volts) that may lead to the aforementioned excess heat, breakdown, and steam generation.

In some variations, an electrode array of a pulsed electric field device may generate an electric field strength of from about 400 V/cm to about 1500 V/cm, from about 1500 V/cm to about 4500 V/cm, including all values and sub-ranges in-between, at a treatment depth of from about 0.5 mm to about 1.5 mm from an inner surface of the duodenum, for example, at about 1 mm. For example, in some variations, the pulsed electric field may comprise an electric field strength (i.e., magnitude) between about 2000 V/cm to about 4500 V/cm, about 3000 V/cm to about 4500 V/cm, about 3500 V/cm to about 4500 V/cm, about 3750 V/cm to about 4250 V/cm, or about 3900 V/cm to about 4100 V/cm, including about 2000 V/cm, about 3000 V/cm, about 3500 V/cm, about 3750 V/cm, about 3900 V/cm, about 4000 V/cm, about 4100 V/cm, about 4250 V/cm, and about 4500 V/cm.

In some variations, the electric field may decay such that the electric field strength is less than about 400 V/cm at about 3 mm from the inner surface of the duodenum. In some variations, a predetermined bipolar current and voltage sequence may be applied to an electrode array of the pulsed electric field device to generate the pulsed or modulated electric field. The generated pulsed or modulated electric field may be substantially uniform to robustly induce cell lysis in a predetermined portion of duodenal tissue. For example, a generated pulsed or modulated electric field may spatially vary up to about 20% at a predetermined depth of tissue, between about 5% and about 20%, between about 10% and 20%, and between about 5% and about 15%, including all ranges and sub-values in-between. Furthermore, the pulsed electric field device may be biocompatible and resistant to stomach acids and intestinal fluids.

As described in detail herein, a tissue treatment device may comprise an expandable member having an uninflated delivery configuration and an inflated treatment configuration. In some variations, the expandable member may comprise or may otherwise be formed from an electrode array (e.g., a plurality of electrodes). In some variations, the expandable member may comprise a flex circuit comprising a plurality of electrodes (e.g., electrode array). The flex circuit may comprise an electrode array or a plurality of electrodes, for example, a plurality of elongate, parallel electrodes. In some variations, the substrate of the electrode array may define one or more apertures (e.g., fluid openings) configured to generate suction (e.g., negative pressure) and/or output fluid (e.g., saline) between adjacent electrodes. The use of suction or negative pressure applied through the openings may draw tissue toward the electrode array and may facilitate contact between the tissue and the electrode array (e.g., may increase a contact area between the surface of the tissue and the electrode surface). For example, the electrode array may be engaged to the tissue via suction through the one or more apertures that may promote more reliable (e.g., consistent) electrical contact between the tissue treatment device and tissue, and therefore a more uniform electric field and an improvement to treatment outcomes. Furthermore, the applied suction may be configured to secure tissue apposition to the electrode array in a uniform manner. In some variations, a plurality of apertures (e.g., row of openings) may be disposed between each pair of proximate (e.g., immediately adjacent) electrodes with a predetermined spacing. For example, the apertures may be spaced apart along a length of an electrode. In some variations, the aperture may be disposed closer to one of the electrodes to promote contact between the tissue and at least one of the electrodes. Additionally or alternatively, the apertures may be disposed equally between proximate electrodes and/or through one or more electrodes. In some variations, the apertures may be disposed along a perimeter of the substrate to facilitate bonding of the substrate to an expandable member and a bonding layer through the apertures. The apertures may be spaced apart from each other and from an edge of the substrate by a minimum of about 0.005 inches.

In some variations, the electrode array may comprise a surface area between about 4 square centimeters and about 42 square centimeters, between about 6 square centimeters and about 10 square centimeters, between about 4 square centimeters and about 8 square centimeters, between about 20 square centimeters and about 42 square centimeters, between about 30 square centimeters and about 42 square centimeters, between about 10 square centimeters and about 30 square centimeters, and between about 8 square centimeters and about 42 square centimeters, including all ranges and sub-values in-between. In some variations, the expandable member in the unexpanded configuration may have an outer diameter between about 15 mm and about 20 mm, including all ranges and sub-values in-between.

Additionally or alternatively, the apertures may be configured for fluid (e.g., gas, fluid) irrigation. The electrode array may be in fluid communication with (e.g., fluidically coupled to) a fluid source (e.g., fluid source of saline, negative pressure source) for fluid irrigation and/or fluid cooling. For example, fluid may be removed from (e.g., suctioned out of) a body cavity or lumen after applying the pulsed or modulated electric field using the electrodes. In some variations, removal of the fluid may facilitate apposition and/or contact between the tissue and the electrode array.

In some variations, at least one of the electrodes may comprise a semi-elliptical cross-sectional shape. In some instances, all of the electrodes in the electrode array may comprise a semi-elliptical cross-sectional shape. Generally, electric fields are intense near points and edges of electrodes due to the high concentration of surface charges there. Sharp-edged electrodes and high electric fields may generate one or more of electric discharge (e.g., arcing), high heat rates (e.g., boiling), high current density (e.g., electrolysis), and bubbles. The semi-elliptical cross-sectional shapes described herein may reduce one or more of these effects relative to sharp-edged electrodes. In some variations, a major axis of the electrode is twice the electrode width and the minor axis of the electrode is equal to the electrode height in the middle of the electrode.

The electrode arrays described herein may be formed using any suitable manufacturing technique. The electrode arrays described herein may be manufactured using any suitable technique including, but not limited to, deposition of solder or other metal, dimpling of the substrate, plating of a metal (e.g., gold), and lamination. In some variations, additional layers and/or coatings may be applied to the electrode.

In some variations, a drive voltage applied to the electrode array may depend at least on the spacing between electrodes of the electrode array as well as electrode dimensions. For example, relatively wide elongate electrodes may reduce the effect of strong electric field intensities at sharply curved edges.

Additionally or alternatively, the plurality of elongate electrodes may comprise an interdigitated configuration. For example, the plurality of elongate electrodes may comprise a curved shape (e.g., S-shape, W-shape). The electrode array may be configured to modify a flexural stiffness of the expandable member to facilitate consistent expansion and compression of the expandable member. In some variations, the electrode array may comprise a plurality of electrodes configured to protrude and/or recess relative to a surface of the substrate.

In some variations, a more uniform treatment of tissue (e.g., in areas where the electrode groups intersect) may be obtained by reducing the widths of the end-most electrodes of each section and reducing the distance between those electrodes. In some variations, a more uniform treatment of tissue (e.g., in areas where the electrode sections intersect) may be enabled by interdigitating the end-most electrodes of each group to overlap the treatment areas.

In some variations, an electrode array may comprise a plurality of electrode sections (e.g., zones), including 1, 2, 3, 4, 5, 6, 7, 8, 9, and 10 or more electrode sections. In some variations, each section of the plurality of sections may comprise a plurality of electrodes. For example, each section of the plurality of sections may comprises between 10 and 18 electrodes. In some variations, an electrode section of an electrode array may have a surface area of between about 250 mm² and about 1000 mm², between about 250 mm² and about 750 mm², between about 500 mm² and about 1000 mm², between 400 mm² and about 500 mm² and between 400 mm² and about 600 mm², including all ranges and sub-values in-between.

In some variations, the electrode array may be configured to generate a substantially uniform electric field at a predetermined tissue treatment depth across its entire surface. For example, a predetermined tissue depth may be configured to receive a voltage field of about 2,500 V/cm. A voltage of about 600 V with a current of about 50 A and a frequency of about 350 kHz may be applied at the electrodes. This may improve the consistency of energy delivery and treatment outcomes.

In some variations, a tissue treatment depth (e.g., 1 mm) receiving about a 2,500 V/cm voltage field may depend on an electrode configuration and the voltage applied to the electrode array. The current may depend on tissue conductivity and electrode configuration. Assuming a constant voltage, an electric field penetration is also constant. The tissue treatment ratio may depend on the state of the tissue during treatment (e.g., stretched, compressed, in-contact with the electrodes). The tissue treatment depth may depend on one or more of a tissue treatment ratio, current, effective voltage, and tissue type.

### Elongate Body

Generally, the elongate bodies (e.g., catheters) of the pulsed electric field devices described herein may be configured to deliver an electrode array to a target tissue for treating the tissue. In some variations, an elongate body may comprise a shaft composed of a flexible polymeric material such as Teflon, Nylon, Pebax, urethane, stainless steel (e.g., coil or braid), nitinol, injection molded plastic, combinations thereof, and the like. In some variations, the pulsed electric field device may comprise one or more steerable or deflectable catheters (e.g., unidirectional, bidirectional, 4-way, omnidirectional). In some variations, the elongate body may comprise one or more pull wires configured to steer or deflect a portion of the elongate body. In some variations, the elongate body may have a bend radius between about 5 cm and about 23 cm and/or between about 45 degrees and about 270 degrees. In some variations, the elongate bodies described herein may comprise a lumen through which another elongate body and/or a guidewire may slide. In some variations, the elongate bodies may comprise a plurality of lumens. For example, the elongate body may comprise one or more of an inflation lumen, fluid lumen, guidewire lumen, and lead lumen.

In some variations, an elongate body may have a length of between about 150 cm and about 200 cm, between about 170 cm and about 200 cm, between about 180 cm and about 190 cm, and between about 150 cm and about 170 cm, including all ranges and sub-values in-between. In some variations, an elongate body may decrease in stiffness proximally to facilitate navigation of the pulsed electric field device through one or more body cavities or lumens. For example, a distal portion of the elongate body may comprise a stiffness of between about 45 D and about 70 D, between about 50 D and about 60 D, and about 55 D, including all ranges and sub-values in-between. The distal portion of the elongate body may have a length of between about 10 inches and about 30 inches, between about 15 inches and about 25 inches, between about 15 inches and about 20 inches, and about 17 inches, including all ranges and sub-values in-between. A proximal portion of the elongate body may comprise a stiffness of between about 50 D and about 100 D, between about 60 D and about 80 D, between about 65 D and about 75 D, and about 70 D including all ranges and sub-values in-between. The proximal portion of the elongate body may have a length of between about 40 inches and about 70 inches, between about 50 inches and about 60 inches, and about 55 inches, including all ranges and sub-values in-between.

In some variations, an elongate body may have a diameter of between about 1 mm and about 20 mm, between about 5 mm and about 15 mm, between about 5 mm and about 10 mm, and between about 10 mm and about 20 mm, including all ranges and sub-values in-between.

In some variations, a lumen of an elongate body may have a diameter of up to about 2 mm, up to about 1.5 mm, up to about 1 mm, up to about 0.5 mm, between about 1 mm and about 2 mm, and between about 1 mm and about 1.5 mm, including all ranges and sub-values in-between.

In some variations, the elongate body may be woven and/or braided and/or coiled, and may be composed of a material (e.g., nylon, stainless steel, nitinol, polymer) configured to enhance pushability, torquability and flexibility. In some variations, the elongate body may comprise a metal-based radiopaque marker comprising one or more of a ring, band, and ink (e.g. platinum, platinum-iridium, gold, nitinol, palladium) configured to permit fluoroscopic visualization. In some variations, the elongate body may comprise magnetic members configured to attract and couple to the visualization device. In some variations, the elongate body may comprise from about 2 layers to about 15 layers of materials to achieve a predetermined set of characteristics.

In some variations, the elongate body and visualization device may be coupled along a predetermined length using one or more of a coupling sleeve, a plurality of rings, and mechanical fasteners. For example, the coupling sleeve may comprise one or more of a polymer sleeve having a spine optionally including scalloped edges, a tubular braid (e.g., Nylon, PET), an inflatable balloon polymer sleeve (e.g., baleeve), and EPTFE biaxially oriented. The plurality of rings may include a chain of rings that may be FEP coated and/or formed of silicone and/or Viton.

In some variations, the elongate body may be an overtube configured to receive the visualization device such that a distal end of the visualization device may be advanced distal to a distal end of the tissue treatment device or through a window in a sidewall of the overtube. In this manner, the overtube may facilitate visualization during delivery of a tissue treatment device to a target treatment site and/or while delivering tissue treatment. For example, visualization allows anatomy to be identified (e.g., ampulla of V ater, proximal/distal edges of the entire treatment area or sections of the treatment area, e.g., for alignment when positioning the tissue treatment device) and for apposition of tissue to a treatment member (e.g., electrode array) to be monitored.

In some variations, the overtube may be more flexible than the visualization device to promote steerability of the tissue treatment device. For example, the overtube may comprise a stiffness of about 0.1 times to about 10 times a stiffness of a visualization device having a diameter configured to be disposed within the tissue treatment device.

In some variations, one or more windows disposed along a sidewall of an overtube may facilitate visualization of tissue and an expandable member. For example, a window of an overtube may be disposed adjacent (e.g., immediately adjacent) and proximal to the expandable member to about 15 cm, about 0.1 mm to about 15 cm, about 0.1 mm to about 10 cm, about 0.1 mm to about 5 cm, about 0.1 mm to about 3 cm, about 0.1 mm to about 2 cm, about 0.1 mm to about 1 cm, about 0.1 mm, about 0.5 mm, about 1 mm, about 2 mm, about 5 mm, about 1 cm, about 1.5 cm, about 2 cm, about 3 cm, about 5 cm, including all ranges and sub-values therebetween. The window may comprise a width of at least an outer diameter of the visualization device to an inner diameter of the overtube, and a length greater than the width such as about 3 cm, about 4 cm, about 5 cm, about 6 cm, about 7 cm, about 8 cm, about 9 cm, and about 10 cm, including all ranges and sub-values therebetween. The window may comprise any suitable shape including a circle, an oval, an ellipse, and a polygon (e.g., a rectangle, a square). The edges of the window may further be atraumatic (e.g., rounded, smoothed). The overtube may comprise a plurality of windows (one, two, three, four or more) disposed radially around a circumference of the overtube and/or longitudinally along a longitudinal length of the overtube. For example, the windows may be spaced apart circumferentially by about 30 degrees, about 60 degrees, about 90 degrees to about 120 degrees, about 30 degrees to about 60 degrees, about 60 degrees to about 90 degrees, about 90 degrees to about 120 degrees, including all ranges and sub-values therebetween. The windows may be spaced apart longitudinally by about 1 mm and about 10 cm, about 1 mm to about 5 cm, about 1 mm to about 3 cm, about 1 mm to about 2 cm, about 1 mm and about 1 cm, about 1 mm, about 2 mm, about 3 mm, about 4 mm, about 5 mm, about 1 cm, about 1.5 cm, about 2 cm, about 2.5 cm, about 3 cm, about 5 cm, about 10 cm, including all ranges and sub-values therebetween.

In some variations, the overtube may comprise a support (e.g., stiffening element) disposed opposite the window where the support is configured to increase a stiffness of the overtube. For example, the support may comprise one or more of coil reinforcement and braid reinforcement. In some variations, the entire overtube may comprise support such as a braid or a coil to add desirable torque and/or stiffness characteristics. The window area of the overtube may, in some variations, only comprise a wire for support.

In some variations, the tissue treatment device may comprise one or more (e.g., two, three, four or more) additional elongate bodies and/or lumens within the overtube configured to provide one or more of inflation, suction, electrical power, and the like. For example, one or more of a second elongate body or lumen comprising an inflation lumen, a third elongate body or lumen comprising a suction lumen, and a fourth elongate body or lumen comprising a lead wire may be provided separately from the overtube coupled to the expandable member or within the body of the overtube respectively. When separate elongate bodies are utilized, any of, including all of, the second, third, and fourth elongate bodies may be independently disposed within the overtube or may be positioned on and coupled to an outer surface of the overtube. In some variations, one or more of the inflation lumen, suction lumen, lead wire, and the like may be disposed on an opposite side of (e.g., 180 degrees from) the window (2012) of the overtube (2010). As noted above, in some variations, a single overtube (2010) may comprise one or more of an inflation lumen, a suction lumen, a lead wire, a pull wire, and the like. In some variations, a pull wire may be configured to deflect one or more of the overtube (2010), expandable member (2020), and visualization device (2040) disposed therein. In some variations, a series of expandable members (2020) may be coupled to a distal portion of the overtube (2010). For example, the expandable members (2020) may be arranged in a serial manner (e.g., in a daisy chain).

In some variations, the tissue treatment device may comprise a plurality of windows, expandable members, and treatment members. For example, the tissue treatment device may comprise a plurality of expandable members (disposed along a length of an overtube) with a corresponding window disposed between each pair of expandable members. Each expandable member may comprise an independently addressable treatment member.

### Actuator

In some variations, an expandable member of a tissue treatment device may transition configurations by using an actuator that allows improved control over the translation, inflation, and deflation of the expandable member. For example, in variations in which an expandable member (e.g., inflatable balloon) is used, the actuator may comprise a linear slider configured for consistent transmission of translational force from the elongate body to the expandable member. Furthermore, the actuator may be configured to actuate one or more pull wires to deflect (e.g., bend, steer) the elongate body. In some variations, the pull wire coupled to the expandable member may be separate from the elongate body and be disposed through a lumen of the visualization device.

### Sheath

Generally, the sheaths of the tissue treatment systems described here may be configured to assist advancement of one or more portions of a tissue treatment device into and through a body cavity or lumen. In some variations, a sheath may generally be configured to dilate a body cavity or lumen, such as a lumen of a duodenum. The sheath may be atraumatic in shape to minimize any inadvertent or unintended damage and may comprise any shape suitable to enlarge a tissue lumen. For example, in some variations, a sheath may comprise a conical shape comprising a taper of between about 1 degree and about 45 degrees, which may facilitate tissue treatment device and visualization device advancement through a body lumen, such as a portion of the gastrointestinal tract. In some variations, the sheath may comprise PET, PEBA, PEEK, PTFE, silicone, elastomer, PS, PEI, latex, sulphate, barium sulfate, a copolymer, combinations thereof, and the like. In some variations, the sheath may comprise a plurality of materials configured to provide a desired stiffness and compliance along a length of the sheath. The sheath may comprise one or more components configured to facilitate advancement of a guidewire.

In some variations, the sheath may comprise a length of between about 2 mm and about 10, between about 2 mm and about 8 cm, between about 2 mm and about 5 cm, between about 1 cm and about 8 cm, between about 1 cm and about 5 cm, between about 3 cm and about 8 cm, between about 3 cm and about 5 cm, between about 5 cm and about 10 cm, including all ranges and sub-values in-between. In some variations, the sheath may comprise a taper of between about 5 degrees and about 30 degrees relative to a longitudinal axis of the sheath. Furthermore, a distal end of the sheath may be atraumatic (e.g., rounded, blunted). In some variations, a tissue treatment device may comprise a plurality of sheaths (e.g., 2, 3, 4, 5, 6, or more). In some variations, the sheath may comprise a shore A hardness of between about 30 Shore A and about 90 Shore A, and between about 40 Shore A and about 80 Shore A, including all ranges and sub-values in-between. In some variations, the sheath may be rigid, such as when a length of the sheath is about the length of an expandable member.

In some variations, a tissue treatment device may comprise one or more sheaths configured to aid advancement of the device through one or more tortuous body cavities without damaging tissue. In some variations, a sheath may comprise a recess configured to facilitate mating or coupling with another elongate member such as a visualization device (e.g., endoscope). For example, this may enable the sheath and expandable member to removably couple to a visualization device during a treatment procedure.

### Handle

Generally, the tissue treatment device devices described herein may comprise a handle configured to allow an operator to grasp and control one or more of the position, orientation, and operation of a tissue treatment device. In some variations, a handle may comprise a grip (e.g., hand grip) and one or more actuators to permit translation and/or rotation of the first and second elongate bodies in addition to steering by an optional delivery catheter. For example, the actuator may comprise one or more of a button, gear, slide, knob, switch, and the like. For example, a slide may be actuated in a distal direction to translate an expandable member and electrode array distal to a sheath and visualization device. Control of an expandable member, in some variations, may be performed by an expansion member (e.g., screw/rotation actuator, inflation actuator) of the handle. In some variations, the handle may be configured to control PEF energy delivery to the electrode array of an expandable member, using, for example, a handheld switch, and/or footswitch. In some variations, the handle may comprise one or more biocompatible polymers, thermoplastics, stainless steel, nitinol, metal fasteners, and lead wires. The handle may be formed by injection molding.

### Insulator

Generally, the tissue treatment devices described herein may include one or more insulators configured to electrically isolate one more portions of the electrode array, expandable member, inflatable member, sheath, and/or elongate body of the tissue treatment device from each other. In some variations, the insulator may comprise one or more of a poly(p-xylylene) polymer such (e.g. parylene C, parylene N), polyurethane (PU), polytetrafluoroethylene (PTFE), expanded PTFE (ePTFE), ETFE, polyimide (PI), polyester, polyethylene terephthalate (PET), PEEK, polyolefin, silicone, copolymer, a ceramic, combinations thereof, and the like. \

### Guidewire

Generally, the systems described herein may comprise one or more guidewires configured to be slidably disposed within a lumen of an elongate body of a tissue treatment device. The guidewire may be configured to assist in advancement of the tissue treatment device through a gastrointestinal tract. In some variations, an elongate body of the tissue treatment device may be translated along the guidewire relative to one another and/or the duodenum. In some variations, the guidewire may comprise one or more of stainless steel, nitinol, platinum, and other suitable biocompatible materials. In some variations, the guidewire may comprise a variable stiffness along its length. For example, a distal tip may be configured to be compliant (e.g., floppy) and an elongate body of the guidewire may be relatively stiff to aid pushability through patient anatomy. In some variations, a guidewire may comprise a diameter between about 0.36 mm and about 1.53 mm, and a length between about 180 cm and about 360 cm.

### Irrigation

Generally, the tissue treatment procedures using a tissue treatment device as described herein may optionally comprise fluid delivery (e.g., fluid irrigation) during tissue treatment. In some variations, the tissue treatment procedures may benefit from fluid irrigation that may promote more reliable (e.g., consistent) electrical contact between the tissue treatment device and tissue and therefore a more uniform electric field and an improvement to treatment outcomes. Fluid irrigation to tissue may further reduce tissue temperature through forced convention and may reduce arcing. Furthermore, fluid delivery may reduce the accumulation of electrically insulating corrosion and electrolysis products. In some variations, the fluid may function as a salt bridge between the electrodes and tissue that allows control of resistivity. In variations in which fluid is delivered, the fluid may be removed from (e.g., suctioned out of) a body cavity after applying the pulsed or modulated electric field. In some variations, the conductivity of the fluid introduced or removed may have an effect on the delivered therapy. For example, adding a solution that is less conductive than the tissue may facilitate more current being introduced into the tissue. Conductivity that is about the same as the tissue may facilitate a transfer of electric field energy into the tissue even if tissue contact between the electrodes and tissue is lacking. Finally, a fluid having a higher conductivity than the tissue may be removed.

In some variations, the tissue treatment devices described herein may be configured to output fluid to irrigate tissue, such as duodenal tissue, of a patient. For example, an electrode array of a tissue treatment device may engage the duodenum and may be configured to output fluid (e.g., saline), for example, where the electrodes contact tissue. The electrode array, for example, one or more electrodes of the electrode array, may output fluid between the electrode and tissue, which may directly target the electrodes and may allow a reduction in fluid volume. The electrode array may be energized to treat a predetermined portion of tissue to resurface the duodenum. Utilizing an electrode array that is configured to deliver fluid may eliminate the need for a separate irrigation device and/or system.

### Sensor

In some variations, the tissue treatment devices and systems described here may comprise one or more sensors. Generally, the sensors may be configured to receive and/or transmit a signal corresponding to one or more parameters. In some variations, the sensor may comprise one or more of a temperature sensor, imaging sensor (e.g., CCD), pressure sensor, electrical sensor (e.g., impedance sensors, electrical voltage sensor, magnetic sensor (e.g., RF coil), proximity sensor, electromagnetic sensor (e.g., infrared photodiode, optical photodiode, RF antenna), force sensor (e.g., a strain gauge), flow or velocity sensor (e.g., hot wire anemometer, vortex flowmeter), acceleration sensor (e.g., accelerometer), chemical sensor (e.g., pH sensors, protein sensor, glucose sensor), oxygen sensor (e.g., pulse oximetry sensor), audio sensor, sensor for sensing other physiological parameters, combinations thereof, and the like. In some variations, the electrical properties of cells can also be determined by applying an alternating current signal at a specific frequency to measure voltage.

Temperature measurements performed during a tissue treatment procedure may be used to determine one or more of tissue contact (e.g., complete contact, partial contact, no contact) with a tissue treatment device and successful energy delivery to tissue. Thus, the safety of the tissue treatment procedures described herein may be enhanced through temperature measurement and monitoring. In some variations, temperature monitoring of the tissue may be used to prevent excess energy delivery to tissue that may otherwise lead to poor or suboptimal treatment outcomes. For example, energy delivery may be inhibited or delayed when tissue temperature measurements exceed a predetermined threshold.

### Suction Catheter

Generally, the suction catheters described herein may be configured to provide suction of tissue to an electrode array while facilitating visualization of the tissue and expandable member during a treatment procedure. For example, the suction catheter may be used with conventional visualization devices (e.g., endoscopes) and provide negative pressure (e.g., suction) through a suction lumen. The expandable member may comprise an electrode array and one or more fluid openings where the suction catheter may be configured to apply suction to tissue through the one or more fluid openings of the expandable member. The expandable member may comprise an expandable member lumen in an expanded configuration. The suction catheter may be configured to be received within the expandable member lumen to assist in applying suction through the one or more fluid openings of the expandable member. Accordingly, tissue contact with the expandable member may be improved while facilitating visualization of the procedure to ensure safety. For example, a suction catheter may be configured to advance from a distal end of a visualization device (e.g., endoscope) into a lumen between the electrode array and expandable member to provide efficient suction between the tissue and electrode array while providing visualization of the tissue and tissue treatment device with a predetermined field-of-view. In some cases, the visualization device may be maintained in place relative to the expandable member when suctioning is performed.

Additionally or alternatively, the tissue treatment device may be configured to provide suction of tissue to an electrode array of an expandable member. For example, the tissue treatment device may comprise a suction catheter coupled (e.g., using a clip, sheath) to the elongate body where the suction catheter is generally disposed parallel to the elongate body. The suction catheter may be configured to be within a lumen of the sheath or alongside an outer surface of the sheath. In some variations, the elongate body of the tissue treatment device may comprise a suction lumen configured to provide suction of tissue to an electrode array of an expandable member. For example, the elongate body may comprise an inner shaft and an outer shaft disposed around the inner shaft, where suction is provided through the outer shaft.

### Multiplexor

Generally, the multiplexors described herein may be configured to provide energy (e.g., PEF energy waveforms) to a tissue treatment device to treat target tissue. For example, a signal generator as described herein throughout may comprise, or be operatively coupled to, a multiplexor configured to distribute a pulsed or modulated electric field waveform generated by the signal generator to one or more sections (e.g., zones, portions, groups, subsets) of the electrode array.

In some variations, the multiplexor may comprise an electric circuit including one or more switches. One or more of the switches may be electrically coupled to predetermined sections of the electrode array. For example, the multiplexor may be configured to independently actuate each switch. Accordingly, the multiplexor may be configured to deliver an electrical signal (e.g., pulse waveform) to a predetermined section of the electrode array. In some variations, the multiplexor may be configured to provide a pulse waveform to one or more sections of the electrode array simultaneously. In some variations, the multiplexor may be configured to provide a pulse waveform to one or more sections of the electrode array asynchronously. For example, the pulse waveform may be delivered asynchronously to one or more sections according to a predetermined sequence.

In some variations, the predetermined sequence may be modified based on the number of sections that in contact with tissue. For example, the expandable member may be configured to expand radially until the expandable member reaches a diameter that corresponds to a diameter of a body cavity or lumen, such as, for example, the GI tract (e.g., duodenum). The expandable member may continue expanding radially to dilate (e.g., stretch) the tissue, such as, for example, a predetermined amount. In some variations, expansion of the expandable member may be limited to prevent damage to tissue due to dilation. The dimensions of a body cavity or lumen (e.g., diameter, tissue thickness) may vary between patients. Therefore, the number of sections of the electrode array that may be in contact with tissue when in an expanded configuration may vary between patients. In some variations, a section of the electrode array may not be included in a predetermined sequence if substantially all of the electrodes of the section are not in contact with tissue. Activating a section of the electrode array with substantially all of the electrodes in contact with tissue may induce electrical shorting. Accordingly, the number of sections included in the predetermined sequence may vary.

In some variations, the predetermined sequence may be modified to ensure that only the sections of the electrode array that are in substantial (e.g., full) contact with tissue are activated. For example, a visualization device may be used to visualize the expandable member, the electrode array and/or tissue as the expandable member transitions from an unexpanded configuration to an expanded configuration. In some variations, an impedance measurement of the electrode array may be used to determine the presence of substantial, or sufficient, tissue contact with the electrodes. In some variations, the pulse generator may measure the impedance on the circuit formed between the pulse generator and the electrodes to determine the presence of substantial, or sufficient, tissue contact with the electrodes. In some variations, an inner surface (e.g., surface opposite a tissue-facing surface) of the expandable member may comprise one or more fiducial markers corresponding to predetermined sections of the electrode array (e.g., section 1, section 2, section 3, section 4, section 5). In some variations, the impedance at the electrodes and/or in the circuit between the pulse generator and the electrodes may be used to determine whether the expandable member and/or pleat(s) are in a folded or an unfolded configuration.

In some variations, the expandable member may comprise an electrode array with one or more electrode sections. For example, the electrode array may comprise between about one and about ten sections, between about one and about eight sections, between about one and about seven sections, between one and about six sections, between about one and about five sections, between about two and about five sections, and between about three and about five sections, including about one section, about two sections, about three sections, about four sections, about five sections, about six sections, about seven sections, about eight sections, about nine sections, and about ten sections, including all ranges and sub-values in-between.

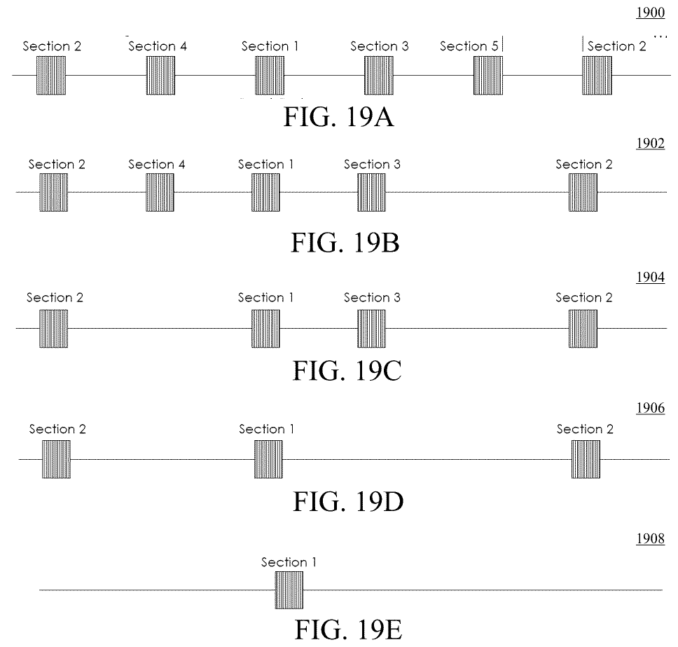

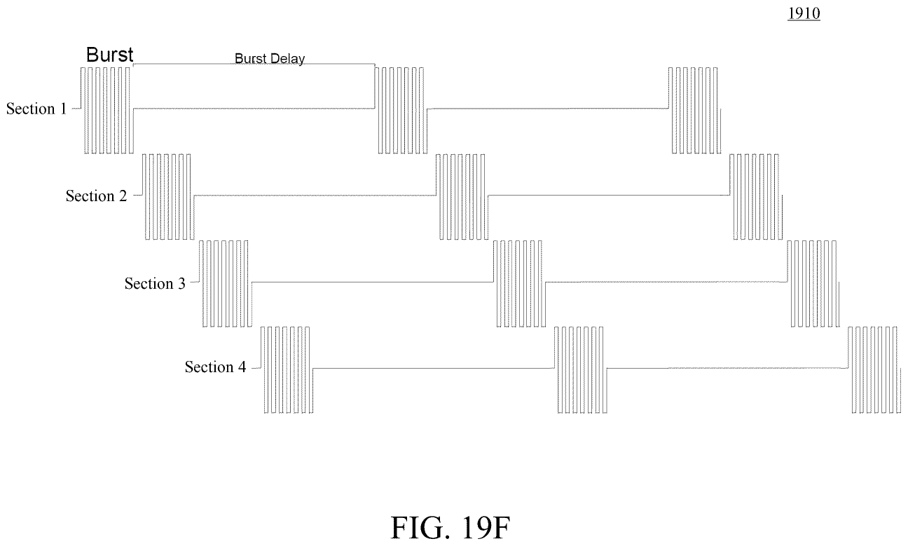

As shown in the schematic diagram (1900) of FIG. 19A, Section 2 of an electrode array may be activated, followed subsequently by activation of Section 4, Section 1, Section 3, and Section 5 in an interleaved manner. The order of the electrode sections of the electrode array may be numerical (e.g., 1, 2, 3, 4, 5) such that Section 1 is proximate to Section 2 (e.g., shares a boundary or edge, in contact with) and non-proximate to Sections 3, 4, and 5 (e.g., separate from, not in contact with). Similarly, Section 2 is proximate to Sections 1 and 3, and non-proximate to Sections 4 and 5. In some variations, deactivation of each section of a plurality of sections is applied independently. For example, one or more sections are not selected for energy delivery in diagrams (1902, 1904, 1906, 1908, 1910) of respective FIGS. 19B, 19C, 19D, 19E, and 19F where the predetermined sequence otherwise follows that of the diagram (1900) of FIG. 19A. For example, section 5 is not activated in FIG. 19B while the timing of energy delivery to sections 2, 4, 1, and 3 is unaffected. In FIG. 19C, sections 4 and 5 are not activated, but the timing of activation of sections 2, 1, and 3 is the same as in FIG. 19A. FIG. 19F further illustrates in the diagram (1910) that energy delivery (e.g., burst, burst delay) to each section is independent of the other sections such that energy delivery is interleaved. That is, the section burst delay is independent of the number of activated electrode sections such that the start of the second pulse waveform does not depend on the number of activated electrode sections. In this manner, the section burst delay for a section may not change even when one or more sections are not selected for activation. In some variations, the plurality of sections may comprise up to about ten sections. In some variations, the signal generator may be configured to repeat the predetermined sequence between about 5 and about 15 times.

### Signal Generator

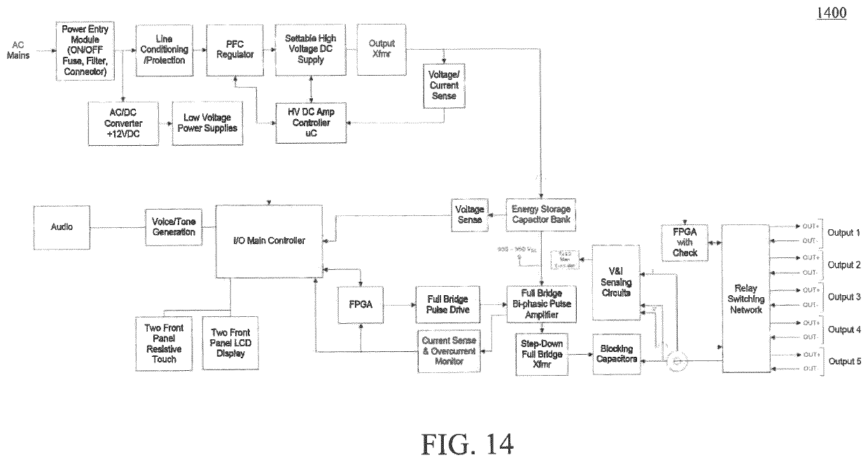

Generally, the systems described herein may include one or more signal generators configured to generate and deliver energy (e.g., PEF energy waveforms) to a tissue treatment device to treat tissue. In some variations, a PEF system as described herein may include a signal generator having an energy source and a processor configured to deliver a waveform to deliver energy to tissue. In some variations, the signal generator may be configured to generate and deliver a plurality of signal types including, but not limited to, AC current, square wave A C current, sine wave A C current, A C current interrupted at predetermined time intervals, multiple profile current pulses trains of various power intensities, direct current (DC) impulses, stimulus range impulses, hybrid electrical impulses, combinations thereof, and the like. For example, the signal generator may be configured to generate one or more monophasic (DC) pulses and biphasic (DC and AC) pulses. FIG. 14 depicts a block diagram (2000) of an exemplary signal generator.

In some variations, a signal generator may be configured to generate a waveform between about 1 V and about 3,000 V, between about 100 V and about 2,000 V, between about 300 V and about 1,000 V, between about 500 V and about 900 V, between about 600 V and about 850 V, between about 715 V and about 825 V, between about 725 V and about 775 V, and between about 740 V and about 760 V, including about 100 V, about 200 V, about 300 V, about 400 V, about 500 V, about 600 V, about 700 V, about 750 V, about 800 V, about 1,000 V, about 1,100 V, about 1,200 V, about 1,300 V, about 1,400 V, about 1,500 V, about 1,600 V, about 1,700 V, about 1,800 V, about 1,900 V, about 2,000 V, including all ranges and sub-values in-between.

In some variations, the pulsed waveform may comprise a drive voltage at the electrode array between about 400 V and about 600 V, between about 400 V and about 550 V, between about 440 V and about 600 V, or between about 440 V and about 550 V, between about 5 kV and about 500 KV, between about 5 kV and about 15 kV, between about 5 kV and about 20 kV, between about 10 kV and about 20 kV, between about 15 kV and about 20 kV, including all values and sub-ranges in-between any of the aforementioned ranges.

In some variations, the pulsed waveform may produce a current through the tissue between about 0.6 A and about 100 A, between about 1 A and about 75 A, between about 20 A and about 60 A, between about 30 A and about 50 A, or between about 36 A and about 48 A from the electrode array per square centimeter of the tissue, including all values and sub-ranges in-between any of the aforementioned ranges.

In some variations, the pulsed waveform may produce a pulsed or modulated electric field at the tissue, including all values and sub-ranges in-between any of the aforementioned ranges. In some variations, the pulsed waveform may comprise a pulse width between about 0.5 μs and about 4 μs, between about 0.1 ns and about 1000 ns, between about 1 ns and about 100 ns, between about 1 ns and about 500 ns, between about 500 ns and about 1000 ns, between about 200 ns and about 800 ns, between about 400 ns and about 600 ns, including all values and sub-ranges in-between any of the aforementioned ranges.

As another example, the pulsed waveform in some variations may comprise a drive voltage at the electrode array between about 5 kV and about 500 kV, between about 5 kV and about 15 kV, between about 5 kV and about 20 kV, between about 10 kV and about 20 kV, between about 15 kV and about 20 kV, including all values and sub-ranges in-between any of the aforementioned ranges. In some variations, the pulsed waveform may comprise an amplitude of at least 10 kV/cm.

In some variations, a signal generator may be configured to generate a waveform having a current delivered into a system resistance of between about 1 A and about 200 A, between about 2Ω and about 30Ω, between about 5Ω and about 20Ω, between about 10Ω and about 20Ω, between about 11Ω and about 18Ω, and between about 12.75Ω and about 16.25Ω, including all ranges and sub-values in-between. For example, in some variations, the system resistance may be about 2Ω, about 5Ω, about 10Ω, about 11Ω, about 12Ω, about 12.75Ω, about 13Ω, about 14Ω, about 14.5Ω, about 15Ω, about 16Ω, about 16.25Ω, about 17Ω, and about 18Ω, including all ranges and sub-values in-between.

In some variations, a signal generator may be configured to generate a waveform having a frequency of between about 50 kHz and about 950 kHz, between about 100 kHz and about 900 kHz, between about 200 kHz and about 800 kHz, between about 300 kHz and about 800 kHz, between about 400 kHz and about 800 kHz, between about 500 kHz and about 800 kHz, between about 600 kHz and about 800 kHz, and between about 700 kHz and about 800 kHz, between about 0.1 Hz and about 10,000 Hz, between about 1 Hz and about 1,000 Hz, between about 1 Hz and about 100 Hz, between about 100 Hz and about 1,000 Hz, between about 1,000 Hz and about 5,000 Hz, between about 5,000 Hz and about 10,000 Hz, between about 2,000 Hz and about 8,000 Hz, and between about 4,000 Hz and about 6,000 Hz, including all values and sub-ranges in-between any of the aforementioned ranges.

It should be appreciated that any combination of energy parameters as disclosed herein may be used. For example, the pulsed waveform in some variations may comprise a frequency between about 50 kHz and about 950 kHz or between about 300 kHz and about 400 kHz, a drive voltage at the electrode array between about 400 V and about 600 V or between about 440 V and about 550 V, and produces a current through tissue between about 36 A and about 48 A from the electrode array per square centimeter of the tissue. The pulsed or modulated electric field at the tissue may be between about 2,000 V/cm and about 3,000 V/cm. In some variations, the pulsed waveform may comprise a set of about 50 pulses in groups of between about 8 and about 13, with a delay of between about 4 seconds and about 10 seconds between each group. In some variations, the pulsed or modulated electric field may be a therapeutic electric field at a compressed tissue depth of between about 0.25 mm and about 0.75 mm and/or an uncompressed tissue depth of between about 0.50 mm and about 1.5 mm. In some variations, the pulse waveform may comprise a pulse width between about 0.5 μs and about 4 μs.

Generally, more than about 1,000 V/cm to about 2,500 V/cm is required at a treatment depth of tissue to induce electric fields across cell membranes greater than about 0.5 V in tissue such as the duodenum. In some variations, more than about 1,500 to about 4,500 V/cm, including all ranges and sub-values in-between, is required at a treatment depth of tissue to induce electric fields across cell membranes greater than about 0.5 V in the duodenum. Even relatively low tissue conductivity (e.g., about 0.3 S/m) may generate bulk tissue heating rates of at least about 800° C./s. The maximum temperature rise that should occur may be about 8° C. such that a maximum continuous on-time (100% duty cycle of alternating polarity pulses) may be about 10 msec. For example, the pulse waveform may comprise pairs of unipolar pulses of about 1 μs in groups between about 5 and about 500, with a delay between each group. In some of these variations, the pulse waveform may comprise a group delay between about 10 μs and about 4000 μs, and an intersection delay (e.g., replenish rate) of between about 50 ms and about 4000 ms, including all ranges and sub-values in-between. In some variations, a series of these groups may be repetitively applied with increasingly longer delays between series. In some variations, a sequence of series may be applied with longer delays between sequences. In some variations, about 15 milliseconds of cumulative ON time may be distributed across about 10 seconds.

In some variations, the signal generator may be configured to generate a waveform having a current, a voltage, and a power in the pulsed or modulated electric field spectrum between about 250 kHz and about 950 M Hz, a pulse width between about 0.5 μs and about 4 μs, a voltage applied by the electrode array of between about 100 V and about 2 kV, and a current density between about 0.6 A and about 100 A from the electrode array per square centimeter of tissue. In some variations, the signal generator may be configured to drive into tissue resistance of from about 5Ω to about 30Ω of load. For example, the current density may be between about 0.6 A and about 100 A from the electrode array per square centimeter of tissue.

In some variations, the pulse waveform may comprise a pulse group of between about 1 and about 50, between about 1 and about 25, between about 1 and 10, between about 5 and 45, between about 10 and 40, between about 20 and 30, and between about 30 and 50, including all ranges and sub-values in-between. Each group of pulses may have between about 1 pulse and about 500 pulses, between about 1 pulse and about 100 pulses, between about 1 pulse and about 200 pulses, between about 1 pulse and about 300 pulses, between about 1 pulse and about 400 pulses, between about 100 pulses and about 500 pulses, between about 200 pulses and about 500 pulses, between about 300 pulses and about 500 pulses, between about 400 pulses and about 500 pulses, and between about 400 pulses and about 500 pulses, including all ranges and sub-values in-between. In some variations, the pulsed waveform may comprise between about 5 groups and about 20 groups or between about 8 groups and about 13 groups, including all values and sub-ranges in-between any of the aforementioned ranges. In some variations, the pulsed waveform may comprise a delay between groups of between about 1 second and about 20 seconds, or between about 4 seconds and about 10 seconds, including all values and sub-ranges in-between any of the aforementioned ranges.

In some variations, a set of bipolar pulses may be divided into bursts of bipolar pairs with a time delay between the bursts. This may allow the heat generated at the cell membranes to disperse, allowing additional treatment before the transition from cell lysis to necrosis. The total time that pulsed or modulated electric field is applied to the tissue may determine the density and size of the membrane pores, and the extent that ion flow has altered the contents of a cell. For example, given a tissue thermal diffusivity κ of 0.13 mm²/s and a cell diameter D_{cell} of 10 micron, the thermal diffusion time may be approximated as D_{cell}²/κ=0.8 msec. Thus, applying a pulse burst and then waiting a millisecond allows the temperature to equilibrate across the cell. For example, a balanced bipolar pulse waveform (e.g., within 10%) may reduce sympathetic nerve excitation, which may reduce perceived pain and spontaneous muscle contraction. In some variations, microsecond pulsing between about 1 μs and about 10 μs may generate cell lysis while minimizing nerve stimulation. An electric field distribution produced by short bipolar pulses does not depend as strongly on tissue homogeneity especially in anisotropic areas.

In some variations, the pulse waveform (i.e., pulsed or modulated electric field waveform) may be generated by the signal generator. The pulse waveform may be delivered to an electrode array such that the electrode array may generate an electric field. In some variations, the depth of treatment may be affected by the size and/or spacing of one or more electrodes of the electrode array and parameters of the pulse waveform. For example, the pulse waveform described herein may comprise one or more pulses. Each pulse may comprise a square, a triangle, a rectangle, or any other shape. In some variations, the pulse may comprise a square shape. In some variations, each pulse may comprise a bipolar pulse. Each pulse may comprise a series (i.e., burst) of pulses. For example, the series of pulses may comprise between about 1 pulse and about 500 pulses, between about 10 pulses and about 90 pulses, between about 20 pulses and about 80 pulses, between about 30 pulses and about 70 pulses, between about 40 pulses and about 60 pulses, and between about 45 pulses and about 55 pulses, between about 50 pulses and about 500 pulses, between about 100 pulses and about 500 pulses, between about 200 pulses and about 500 pulses, between about 300 pulses and about 500 pulses, between about 400 pulses and about 500 pulses, between about 10 pulses and about 200 pulses, between about 10 pulses and about 100 pulses, including about 1 pulse, about 10 pulses, about 15 pulses, about 20 pulses, about 30 pulses, about 40 pulses, about 45 pulses, about 50 pulses, about 55 pulses, about 60 pulses, about 70 pulses, about 80 pulses, about 90 pulses, about 100 pulses, including all ranges and sub-values in-between. In some variations, the series of pulses may comprise about 50 pulses.

Pulsed electric field waveforms may be delivered to an electrode array where, for example, two or more non-proximate sections of the plurality of sections of the electrode array receive the waveform in a predetermined sequence (e.g., of different groups of pulses for different sections) in order to increase safety and/or reduce unintended damage to the tissue by reducing a temperature increase in tissue. In some variations, the predetermined sequence described herein may comprise delivering a series of pulses per activation of a given section. Accordingly, the inter-section delay may correspond to the time interval between an end of a first series of pulses delivered to a first section and a start of a second series of pulses delivered to a second section. The intra-section delay may correspond to the time interval between an end of the first series of pulses delivered to a first section and a start of the second series of pulses delivered to the first section.

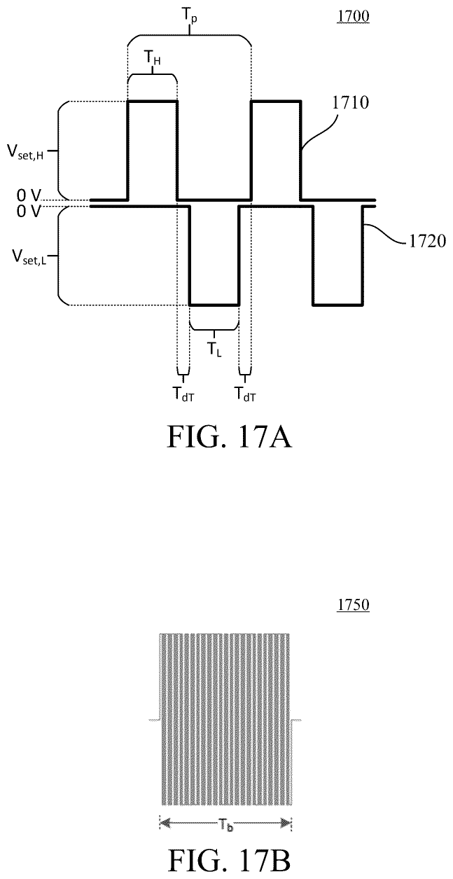

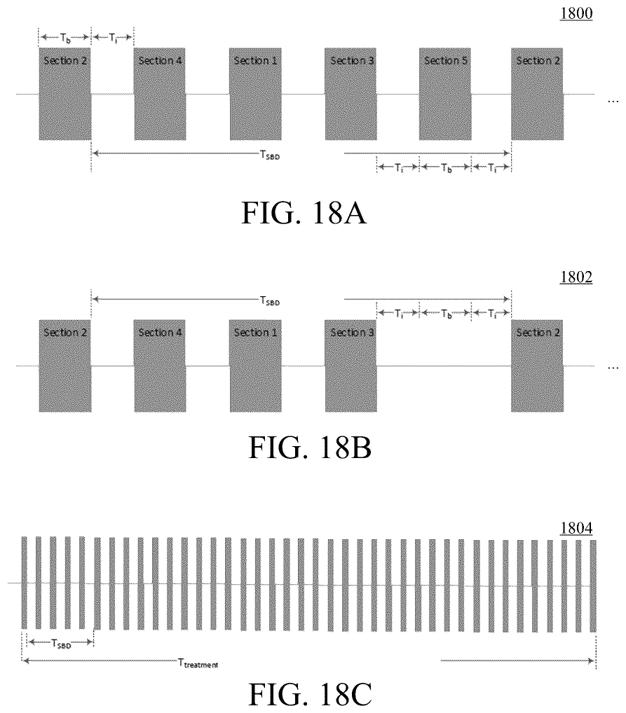

FIG. 17A is a schematic diagram (1700) of a pulse waveform. In some variations, each pulse of a pulse waveform may comprise a pulse width (Tₚ). In some variations, a pulse width Tₚ may correspond to the time between adjacent maxima or minima of a wave. In some variations, the pulse width Tₚ may be measured between zero-crossings that correspond to a full wave. In some variations, a set voltage Vₛₑₜ may correspond to the amplitude of the pulse measured in volts. In some variations, the pulse width may be inversely proportional to a frequency (f) of the pulse waveform, as given by equation (2):

$${T}_{p} = \frac{1}{f} \qquad eqn . ( 2 )$$

As shown in FIG. 17A, the pulse width may include a first time interval (T_{H}) corresponding to a positively-charged portion of a bipolar pulse and a second time interval (T_{L}) corresponding to a negatively-charged portion of a bipolar pulse. In some variations, the first time interval (T_{H}) and second time interval (T_{L}) may be equivalent.

In some variations, the pulse may comprise a square or rectangular shape comprising one or more phases. Each phase may be generated by a portion of an electric circuit, such as an H-bridge, of the signal generator. For example, as shown in FIG. 17A, a pulse may comprise a first phase 1710 (“Phase 1”) and a second phase 1720 (“Phase 2”). The first phase 1710 may be generated by a first portion of the H-bridge and the second phase 1720 may be generated by a second portion of the H-bridge. In some variations, the phases may be generated in parallel, such that net-zero points of a given phase may correspond to a non-zero point of another phase. The net-zero points of a given phase may correspond to an interval of zero energy. The waveforms of each phase may comprise one or more parameters. For example, as illustrated, the phase(s) may comprise the first time interval Tₕ, the pulse width Tₚ, the non-zero time interval T_{dt} (e.g., dead time), and a second time interval T_{L}. The Tₕ value may correspond to the interval of the waveform at a positive maxima. The T_{L} value may correspond to the interval of the waveform at a negative minima. The Tₚ value may correspond to the time between adjacent maxima or minima of a wave.

In some variations, the pulse width may be between about 1 μs to about 10 μs, between about 5 μs to about 10 μs, between about 3 μs to about 7 μs, between about 1.5 μs to about 4 μs, between about 2 μs to about 3.5 μs, between about 2.5 μs to about 3.25 μs, between about 2.7 μs to about 3 μs, and between about 2.8 μs to about 2.9 μs, including 1 μs, about 1.5 μs, about 2 μs, about 2.5 μs, about 2.6 μs, about 2.7 μs, about 2.8 μs, about 2.82 μs, about 2.84 μs, about 2.86 μs, about 2.88 μs, about 2.9 μs, about 3 μs, about 3.1 μs, about 3.25 μs, about 3.5 μs, about 4 μs, or about 5 μs. In an exemplary variation, the pulse width may be about 2.86 μs.

In some variations, the first time interval (T_{H}) and/or the second time interval (T_{L}) may each be between about 0.5 μs and about 2 μs, between about 1 μs and about 2 μs, between about 1.1 μs and about 1.9 μs, between about 1.2 μs and about 1.7 μs, between about 1.3 μs and about 1.5 μs, and between about 1.4 μs and about 1.5 μs, including about 0.5 μs, about 1 μs, about 1.1 μs, about 1.2 μs, about 1.3 μs, about 1.4 μs, about 1.41 μs, about 1.42 μs, about 1.43 μs, about 1.44 μs, about 1.45 μs, about 1.5 μs, and about 1.6 μs, including all ranges and sub-values in-between. In some variations, the first time interval (T_{H}) and the second time interval (T_{L}) may each be about 1.43 μs.

In some variations, the frequency may be between about 50 KHz and about 950 kHz, between about 100 kHz and about 900 kHz, between about 200 kHz and about 500 kHz, between about 300 kHz and about 400 kHz, and between about 325 kHz and about 375 kHz, including about 100 kHz, about 200 kHz, about 300 kHz, about 325 kHz, about 350 kHz, about 375 kHz, about 400 kHz, and about 500 kHz, including all ranges and sub-values in-between. In an exemplary variation, the frequency may comprise about 350 KHz.

As shown in FIG. 17A, the signal generator described herein may be configured to switch from the positive portion to the negative portion with a non-zero time interval (T_{dT}) therebetween comprising a net-zero charge. Advantageously, a non-zero time interval of a net-zero charge may reduce risks associated with electrical shorting and/or electrical cross-talk within one or more components of the signal generator. In some variations, the non-zero time interval (T_{dT}) may be between about 0.01 μs and about 0.1 μs, between about 0.02 μs and about 0.1 μs, between about 0.03 μs and about 0.1 μs, between about 0.04 μs and about 0.1 μs, between about 0.05 μs and about 0.1 μs, between about 0.06 μs and about 0.1 μs, between about 0.07 μs and about 0.1 μs, and between about 0.08 μs and about 0.09 μs, including about 0.01 μs, about 0.02 μs, about 0.03 μs, about 0.04 μs, about 0.05 μs, about 0.06 μs, about 0.07 μs, about 0.08 μs, about 0.085 μs, about 0.086 μs, and about 0.09 μs, including all ranges and sub-values in-between. Alternatively, the pulse waveform may not include a non-zero time interval (T_{dT}). In some variations, the non-zero time interval (T_{dT}) corresponds to the first time interval (T_{H}), second time interval (T_{L}), and pulse width (Tₚ) as given by equation (3).

$${T}_{p} = {T}_{H} + {T}_{L} + 2 {T}_{dT} \qquad eqn . ( 3 )$$

As shown in the schematic diagram (1750) of FIG. 17B, the series or group of pulses described herein may comprise a burst time (T_{b}) or group time representing the time between a start of the series and an end of the series of pulses (e.g., a group of pulses). In some variations, the burst time (T_{b}) may correspond to the pulse width (Tₚ) and a number of pulses (nₚ) per series as given by equation (4) below. As described in more detail herein, a first group of pulses may correspond to a first pulse waveform delivered to a first section of an electrode array, and a second group of pulses may correspond to a second pulse waveform delivered to a second section of an electrode array. As described herein, a series of pulses may be applied to an electrode section of an electrode array. The burst time (T_{b}) may be determined using equation (4).

$${T}_{b} = {n}_{p} * {T}_{p} \qquad eqn . ( 4 )$$

In some variations, the series or group of pulses (e.g., bipolar pulsed electric field waveforms) may comprise between about 1 and about 100 pulses, between about 10 pulses and about 90 pulses, between about 20 pulses and about 80 pulses, between about 30 pulses and about 70 pulses, between about 40 pulses and about 60 pulses, between about 45 pulses and about 55 pulses, between about 40 pulses and about 50 pulses, and between about 50 pulses and about 60 pulses, between about 50 pulses and about 500 pulses, between about 100 pulses and about 500 pulses, between about 200 pulses and about 500 pulses, between about 300 pulses and about 500 pulses, between about 400 pulses and about 500 pulses, between about 10 pulses and about 200 pulses, between about 10 pulses and about 100 pulses, including about 1 pulse, about 10 pulses, about 20 pulses, about 30 pulses, about 40 pulses, about 45 pulses, about 50 pulses, about 55 pulses, about 60 pulses, about 70 pulses, about 80 pulses, about 90 pulses, and about 100 pulses, including all ranges and sub-values in-between. In some variations, the series of pulses may comprise about 50 pulses.

In some variations, one or more of the electrode array sections may be energized (i.e., activated) according to a predetermined sequence using interleaved waveforms. For example, the sections may be activated successively (i.e., one section after another) such that successively activated sections are not proximate (e.g., immediately adjacent) to one another. For example, successively activated sections may be separated by at least one other section of the electrode array. In another example, successively activated sections may be separated by a non-conductive portion. In some variations, the predetermined sequence may comprise activating sections sequentially, such that proximate sections may be activated successively. In some variations, the sections may be wired and/or may be activated independently of one another. Alternatively, two or more sections may be activated concurrently. For example, non-proximate pairs of sections may be activated simultaneously to reduce a treatment time.

As shown in the schematic diagram (1800) of FIG. 18A, Section 2 of an electrode array may be activated, followed subsequently by activation of Section 4, Section 1, Section 3, and Section 5 in an interleaving manner. The section labeling shown in FIGS. 18A and 18B are exemplary and may be arranged in any order. For example, section 1 may be proximate (e.g., immediately adjacent) to section 2, which may be proximate to section 3, and so on. In some variations, the predetermined sequence may include a section burst delay (T_{SBD}), which may correspond to a time interval between the end of a burst of a given section and the beginning of a burst of that same section. In some variations, the section burst delay may be between about 1 second and about 20 seconds, between about 1 second and about 10 seconds, between about 1 second and about 5 seconds, between about 2 seconds and about 8 seconds, and between about 3 seconds and about 5 seconds, including all ranges and sub-values in-between.

In some variations, the predetermined sequence may be modified based on a selection of sections. For example, the predetermined sequence may initially be configured to activate every section (e.g., sections 1-5) of an electrode array. In some variations, a subset of the sections may be selected (e.g., by a user) or pre-programmed and a corresponding predetermined sequence may be modified to optimize the treatment. For example, a portion of tissue to be treated may have a diameter such that the tissue may be optimally dilated with four sections of the electrode array in contact with tissue and the fifth section of the electrode array not in contact with tissue. Accordingly, sections 1-4 of the electrode array may be selected for energy delivery and section 5 may be unselected, such that the unselected section is not activated.

As shown in schematic diagram (1800) of FIG. 18A, a predetermined sequence is shown where Sections 1-5 of an electrode array activated in a predetermined interleaved order (e.g., Section 2, Section 4, Section 1, Section 3, Section 5) using a first pulse waveform, and a second pulse waveform repeats the activation pattern of the first pulse waveform after an intra-section delay (T_{SBD}) (i.e., second delay). The pulse waveforms (e.g., first, second) may include one or more delays. For example, different sections (e.g., Section 2 followed by Section 4) may be activated following an inter-section delay (Tᵢ) (i.e., a first delay, an inter-section delay). In some variations, the delays (Tᵢ, T_{SBD}) may correspond to the burst time (T_{b}) and a number of pulses per series or group or burst (nₚ) as given in equation (5).

$${T}_{i} = \frac{{T}_{SBD} - ( {T}_{b} * ( {n}_{s} - 1 ) )}{{n}_{s}} \qquad eqn . ( 5 )$$

In some variations, the inter-section delay (Tᵢ) between pulse groups may be the same or different (e.g., an inter-section delay between Section 2 and 4 may be different from the inter-section delay between Sections 5 and 2). The intra-section delay (T_{SBD}) between different pulse waveforms may be the same or different.

In some variations, the number of activated sections of an electrode array may be based on one or more of the target tissue and/or chronic condition to be treated. In FIG. 18A, an electrode array includes five sections where every section of the electrode array is activated in the predetermined sequence. FIG. 18B depicts a schematic diagram (1802) where the predetermined sequence activates less than all sections of the electrode array in an interleaved manner. This may be useful where a smaller diameter of the expandable member is sufficient to treat target tissue such that, for example, only four of the five sections of the electrode array are activated and in contact with tissue. In some variations, deactivation of each section of a plurality of sections is applied independently. For example, Section 5 is not selected for energy delivery in diagram (1802) where the predetermined sequence otherwise follows that of the diagram (1800) of FIG. 18A. That is, the section burst delay may be independent of the number of activated electrode sections.

In some variations, activation of one or more sections of an electrode array may provide partial or full circumferential treatment of tissue. For example, a predetermined sequence may treat a circumference of tissue of up to about 360°, of up to about 330°, of up to about 300°, of up to about 270°, of up to about 240°, of up to about 210°, of up to about 180°, of up to about 150°, of up to about 120°, of up to about 90°, of up to about 60°, and of up to about 30°, including all ranges and sub-values in-between.

In some variations, the inter-section delay (Tᵢ) may comprise a time interval between about 10 ms and about 4 seconds, between about 50 ms and about 4 seconds, between about 100 ms and about 2 seconds, between about 200 ms and about 1 second, between about 300 ms and about 900 ms, between about 500 ms and about 850 ms, between about 600 ms and about 850 ms, between about 700 ms and about 850 ms, and between about 750 ms and about 850 ms, including about 50 ms, about 100 ms, about 200 ms, about 300 ms, about 400 ms, about 500 ms, about 600 ms, about 700 ms, about 800 ms, about 850 ms, about 1 second, about 2 seconds, about 3 seconds, and about 4 seconds, including all ranges and sub-values in-between. In some variations, an inter-section delay may comprise a time interval of about 800 ms or less.

In some variations, the intra-section delay may be different (e.g., shorter, longer) than the inter-section delay. In some variations, the intra-section and inter-section delays may be the same. In some variations, the intra-section delay may be between about 1 second and about 10 seconds, between about 1 second and about 8 seconds, between about 2 seconds and about 6 seconds, between about 3 seconds and about 5 seconds, and between about 3.5 seconds and about 4.5 seconds, including about 1 second, about 2 seconds, about 3 seconds, about 4 seconds, about 5 seconds, about 6 seconds, about 7 seconds, about 8 seconds, about 9 seconds, and about 10 seconds, including all ranges and sub-values in between. In some variations, an inter-section delay may be about 800 ms or less, between about 500 ms and about 1000 ms, or between about 500 ms and about 800 ms, including all ranges and sub-values in-between.

FIG. 18C depicts a diagram (1804) of a total treatment sequence including a plurality of pulse waveforms. The time interval from the beginning of the first pulse to the end of the last pulse may correspond to a cumulative treatment time Tₜᵣₑₐₜₘₑₙₜ. In some variations, the signal generator may be configured to activate the plurality of sections for a cumulative activation time between about 0.1 ms and about 10 ms over a treatment time between about 30 seconds and about 35 seconds, including all ranges and sub-values in-between. The total treatment sequence may comprise a plurality of pulse waveforms including up to about 50 pulse waveforms, up to about 40 pulse waveforms, up to about 30 pulse waveforms, up to about 20 pulse waveforms, up to about 15 pulse waveforms, up to about 10 pulse waveforms, and up to about 5 pulse waveforms, including all ranges and sub-values in-between. Any combination of the energy parameters described herein may be used and the treatment may be tailored to the particular target tissue and chronic condition being treated.

In some variations, the signal generator may be configured to control waveform generation and delivery in response to received sensor data. For example, energy delivery may be inhibited when a temperature sensor measurement confirms tissue temperature exceeding a predetermined threshold or ranges (e.g., above a predetermined maximum temperature). For example, energy delivery may be inhibited based on a temperature increase over a predetermined period of time (e.g., an increase of 2° C. over one second of time may inhibit further energy delivery).

In some variations, the signal generator may comprise a processor, memory, energy source (e.g., current source), and user interface. The processor may incorporate data received from one or more of the memory, the energy source, the user interface, and the tissue treatment device. The memory may further store instructions to cause the processor to execute modules, processes and/or functions associated with the system, such as waveform generation and delivery. For example, the memory may be configured to store patient data, clinical data, procedure data, safety data, and/or the like.

Generally, the processor (e.g., CPU) of a signal generator described here may process data and/or other signals to control one or more components of the system. The processor may be configured to receive, process, compile, compute, store, access, read, write, and/or transmit data and/or other signals. In some variations, the processor may be configured to access or receive data and/or other signals from one or more of a sensor (e.g., temperature sensor) and a storage medium (e.g., memory, flash drive, memory card). In some variations, the processor may be any suitable processing device configured to run and/or execute a set of instructions or code and may include one or more data processors, image processors, graphics processing units (GPU), physics processing units, digital signal processors (DSP), analog signal processors, mixed-signal processors, machine learning processors, deep learning processors, finite state machines (FSM), compression processors (e.g., data compression to reduce data rate and/or memory requirements), encryption processors (e.g., for secure wireless data and/or power transfer), and/or central processing units (CPU). The processor may be, for example, a general purpose processor, Field Programmable Gate Array (FPGA), an Application Specific Integrated Circuit (A SIC), a processor board, and/or the like. The processor may be configured to run and/or execute application processes and/or other modules, processes and/or functions associated with the system. The underlying device technologies may be provided in a variety of component types (e.g., metal-oxide semiconductor field-effect transistor (MOSFET) technologies like complementary metal-oxide semiconductor (CM OS), bipolar technologies like emitter-coupled logic (ECL), polymer technologies (e.g., silicon-conjugated polymer and metal-conjugated polymer-metal structures), mixed analog and digital, and/or the like.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (A SIC). Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

Generally, the tissue treatment device described here may comprise a memory configured to store data and/or information. In some variations, the memory may comprise one or more of a random access memory (RAM), static RAM (SRAM), dynamic RAM (DRAM), a memory buffer, an erasable programmable read-only memory (EPROM), an electrically erasable read-only memory (EEPROM), a read-only memory (ROM), flash memory, volatile memory, non-volatile memory, combinations thereof, and the like. In some variations, the memory may store instructions to cause the processor to execute modules, processes, and/or functions associated with a tissue treatment device, such as signal waveform generation, tissue treatment device control, data and/or signal transmission, data and/or signal reception, and/or communication. Some variations described herein may relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for the specific purpose or purposes.

In some variations, the tissue treatment device may further comprise a communication device configured to permit an operator to control one or more of the devices of the PEF system. The communication device may comprise a network interface configured to connect the tissue treatment device to another system (e.g., Internet, remote server, database) by wired or wireless connection. In some variations, the tissue treatment device may be in communication with other devices (e.g., cell phone, tablet, computer, smart watch, and the like) via one or more wired and/or wireless networks. In some variations, the network interface may comprise one or more of a radiofrequency receiver/transmitter, an optical (e.g., infrared) receiver/transmitter, and the like, configured to communicate with one or more devices and/or networks. The network interface may communicate by wires and/or wirelessly with one or more of the tissue treatment device, network, database, and server.

The network interface may comprise RF circuitry configured to receive and/or transmit RF signals. The RF circuitry may convert electrical signals to/from electromagnetic signals and communicate with communications networks and other communications devices via the electromagnetic signals. The RF circuitry may comprise well-known circuitry for performing these functions, including but not limited to an antenna system, an RF transceiver, one or more amplifiers, a tuner, one or more oscillators, a mixer, a digital signal processor, a CODEC chipset, a subscriber identity module (SIM) card, memory, and so forth.

Wireless communication through any of the devices may use any of plurality of communication standards, protocols and technologies, including but not limited to, Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (WiFi) (e.g., IEEE 802.11a, IEEE 802.11b, IEEE 802.11g, IEEE 802.11n, and the like), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol. In some variations, the devices herein may directly communicate with each other without transmitting data through a network (e.g., through NFC, Bluetooth, WiFi, RFID, and the like).

In some variations, the user interface may comprise an input device (e.g., touch screen) and output device (e.g., display device) and be configured to receive input data from one or more of the tissue treatment device, network, database, and server. For example, operator control of an input device (e.g., keyboard, buttons, touch screen) may be received by the user interface and may then be processed by processor and memory for the user interface to output a control signal to the tissue treatment device. Some variations of an input device may comprise at least one switch configured to generate a control signal. For example, an input device may comprise a touch surface for an operator to provide input (e.g., finger contact to the touch surface) corresponding to a control signal. An input device comprising a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies. In variations of an input device comprising at least one switch, a switch may comprise, for example, at least one of a button (e.g., hard key, soft key), touch surface, keyboard, analog stick (e.g., joystick), directional pad, mouse, trackball, jog dial, step switch, rocker switch, pointer device (e.g., stylus), motion sensor, image sensor, and microphone. A motion sensor may receive operator movement data from an optical sensor and classify an operator gesture as a control signal. A microphone may receive audio data and recognize an operator voice as a control signal.

A haptic device may be incorporated into one or more of the input and output devices to provide additional sensory output (e.g., force feedback) to the operator. For example, a haptic device may generate a tactile response (e.g., vibration) to confirm operator input to an input device (e.g., touch surface). As another example, haptic feedback may notify that operator input is overridden by the tissue treatment device.

### II. Methods

Also described here are methods of treating tissue. In some variations, methods may comprise treating diabetes of a patient using the systems and devices described herein. In particular, the systems, devices, and methods described herein may resurface a predetermined portion of tissue, for example, duodenal tissue, for the treatment of, for example, diabetes using a pulsed or modulated (e.g., sine wave) electric field.

Generally, the methods of treating tissue may deliver pulsed or modulated electric field energy to remove native endothelial cell populations through non-thermal cell death that may address metabolic disorders such as, for example, obesity, Non-alcoholic fatty liver disease (NAFLD), Nonalcoholic steatohepatitis (NASH), Type I diabetes, and Type II diabetes. Gastric mucosal devitalization (GMD) without thermal injury to muscularis propria may modify one or more of serum ghrelin levels, triglycerides, HDL, relative weight loss, visceral adiposity, organ lipid content, liver lipid/protein ratio, gluconeogenesis, and liver lipid accumulation. Any of the methods described herein, such as energy delivery, may be performed using a monopolar or bipolar configuration in a body cavity or lumen of the patient such as, for example, an esophagus, a stomach, a large intestine (e.g., cecum, colon, rectum, anal canal), a small intestine, any portion of the gastrointestinal tract, vasculature (e.g., blood vessels), a thoracic cavity (e.g., lungs), an abdomino-pelvic cavity, a pelvic cavity (e.g., bladder), a vertebral cavity, a cranial cavity (e.g., nasal passageway), and the like. For example, energy delivery for treating Barrett's esophagus may provide long-term symptom management and reduce complications such as cancer. In some variations, precancerous esophageal cells may be treated while preserving healthy esophageal tissue.

In some variations, treating the target tissue treats one or more of a metabolic disorder, pre-cancer, cancer, proinflammatory processes, immunological processes. In some variations, the metabolic disorder may comprise one or more of obesity, Non-alcoholic fatty liver disease (NAFLD), Nonalcoholic steatohepatitis (NASH), Type I diabetes, and Type II diabetes. In some variations, the target tissue may comprise one or more of a duodenum, a pylorus, an esophagus, a stomach, a small intestine, and a large intestine. Gastric mucosal devitalization (GMD) without thermal injury to muscularis propria may modify one or more of serum ghrelin levels, triglycerides, HDL, relative weight loss, visceral adiposity, organ lipid content, liver lipid/protein ratio, gluconeogenesis, and liver lipid accumulation. Energy delivery may be performed using a monopolar or bipolar configuration. For example, energy delivery for treating Barrett's esophagus may provide long-term symptom management and reduce complications such as cancer. In some variations, precancerous esophageal cells may be treated while preserving healthy esophageal tissue. Any of the methods described herein may be performed in any portion of a body cavity or lumen of the patient such as, for example, an esophagus, a stomach, a large intestine (e.g., cecum, colon, rectum, anal canal), a small intestine, any portion of the gastrointestinal tract, vasculature (e.g., blood vessels), a thoracic cavity (e.g., lungs), an abdomino-pelvic cavity, a pelvic cavity (e.g., bladder), a vertebral cavity, a cranial cavity (e.g., nasal passageway), and the like.

In some variations, the generated pulsed or modulated electric field may be substantially uniform such that pulsed or modulated electric field energy for tissue treatment may be delivered to a predetermined portion of tissue (e.g., mucosal layer of the duodenum) without significant energy delivery to deeper layers of the duodenum. Thus, the methods may improve the efficiency and effectiveness of energy delivery to duodenal tissue. Moreover, the methods described here may also avoid the excess thermal tissue heating necessarily generated by application of one or more other thermal energy modalities to tissue.

In some variations, methods may include applying suction to a tissue treatment device in contact with tissue using a suction catheter. The suction catheter may, in some variations, be advanced from a lumen of a visualization device. The suction catheter may apply negative pressure to the tissue to further aid in a consistent tissue engagement with the expandable member and improved energy delivery.

In some variations, energy delivery may include activating different sections of the electrode array in a predetermined order to minimize treatment time, an energy dose applied to tissue, and/or a temperature increase in the tissue. For example, non-proximate sections of the electrode array may be activated after an inter-section delay to generate a therapeutic electric field, minimize tissue temperature increase, and reduce electrical cross-talk.

### Method of Treating Tissue

Generally, methods of treating tissue may comprise generating a pulsed or modulated electric field to cause a change in tissue to treat one or more chronic condition, such as, for example, a metabolic disorder, pre-cancer, cancer, proinflammatory processes, immunological processes, and neurological disorders. For example, the metabolic disorder may comprise one or more of obesity, non-alcoholic fatty liver disease (NAFLD), nonalcoholic steatohepatitis (NASH), Type I diabetes, and Type II diabetes. In some variations, the tissue may include tissue from any body lumen or cavity such as any portion of the gastrointestinal tract, vasculature (e.g., blood vessels), a thoracic cavity (e.g., lungs), an abdomino-pelvic cavity, a pelvic cavity (e.g., bladder), a vertebral cavity, a cranial cavity (e.g., nasal passageway), and the like.

Referring now to variations in which the methods described herein treat or reduce one or more symptom of diabetes, normally, the small intestine sends signals to the brain, pancreas, and liver to promote glycemic hemostasis. For example, enteroendocrine cells of the mucosal villa may generate these signals. Duodenal mucosal resurfacing using the systems, methods, and devices described herein may be used to treat, for example, type 2 diabetes. Clinical studies have demonstrated that duodenal mucosal resurfacing of the mucosal layer of the duodenum is a safe procedure that may have a positive impact on glycemic hemostasis in patients with type 2 diabetes.

In some variations, the pulsed or modulated electric field may cause cell lysis in tissue that is at least 50% pore-induced and less than 50% heat-induced. In some variations, a method of treating diabetes may include advancing a tissue treatment device towards a target tissue of a patient. For example, a patient may be positioned on their left lateral side during the procedure, and the target tissue (e.g., duodenum) may optionally be insufflated (e.g., using CO₂ or saline). The tissue treatment device may comprise an elongate body and an expandable member comprising an electrode array. Once in the target tissue (e.g., duodenum), the expandable member may be transitioned into a treatment configuration. In some variations, one or more turns of the expandable member may be unrolled to contact the target tissue. In some variations, a visualization device (e.g., endoscope) may be advanced into the target tissue (e.g., duodenum) to visualize, inspect, and/or confirm a treatment area during a procedure. For example, one or more transparent portions of a tissue treatment device may allow the visualization device to identify a location of the tissue treatment device within patient anatomy (e.g., an ampulla of the duodenum, bulb of the duodenum). Once the device is located at a desired position within the target tissue, a pulse waveform may be delivered to the electrodes to generate a tissue treatment to treat a portion of the target tissue. It should be appreciated that any of systems and devices described herein may be used in the methods described here.

In some variations, a method of treating tissue may include concurrently advancing a visualization device through an overtube of a tissue treatment device coupled to a visualization device in an unexpanded configuration (e.g., delivery configuration) to a target treatment site such that advancement may be performed safely without the need for fluoroscopic guidance, thus reducing the number of procedural steps. When disposed at a target treatment site (e.g., target tissue), the visualization device may be advanced through a window of the overtube to facilitate visualization of the treatment device and/or tissue. Additionally or alternatively, when disposed at a target treatment site, the tissue treatment device may atraumatically transition to a treatment configuration where tissue may contact, but not become unintentionally coupled (e.g., caught, trapped, stuck) to the tissue treatment device. After treating one portion of tissue, additional portions of tissue may be treated by translating and/or rotating the tissue treatment device. Treated portions of tissue may also be re-treated as desired (for example, one, two, three, four, or more times).

In some variations, a method of treating tissue may further include one or more of application of a radially outward force to stretch (e.g., dilate) tissue and application of negative pressure (e.g., suction) to the tissue to facilitate a consistent (e.g., uniform) tissue-electrode interface. For example, tissue stretched or dilated by an expandable member of a tissue treatment device in the treatment configuration, whether through the application of a radial force and/or negative pressure, may have a more uniform tissue thickness, which may aid in a consistent energy delivery and treatment. In some variations, tissue may be in contact with the expandable member in the treatment configuration within the target tissue. A visualization device (e.g., endoscope) may be positioned proximal to the expandable member in the treatment configuration. Optionally, a suction catheter may be advanced from a lumen of the visualization device into a lumen of the expandable member. The suction catheter may be configured to generate a negative pressure sufficient to pull tissue into and/or through one or more openings (e.g., fluid openings) of the expandable member. In some variations, suction may be applied through a lumen (e.g., working channel) of the visualization device and/or of the elongate body (e.g., overtube) of the treatment device. This may reduce tissue tenting and/or air pockets over the electrodes and ensure a consistent tissue-tissue treatment member (e.g., electrode) interface tissue around an inner circumference of the target tissue. Furthermore, suction may enable a reduction in the radial force applied by the expandable member. In some variations, the negative pressure (e.g., suction) applied to the tissue may be between about 50 mmHg and about 75 mmHg. In some variations, the negative pressure (e.g., suction) applied to the tissue may be applied intermittently or in relatively short time periods at a pressure of between about 100 mmHg and about 250 mmHg. For example, higher negative pressure may be applied in spurts or feathered so as to ensure contact between the tissue and the electrodes without tissue pressure necrosis.

Stretched tissue dilated by the expandable member in the expanded configuration may reduce a wall thickness of the tissue, thereby allowing for a lower dose of energy to treat a predetermined depth of tissue. Stretched tissue may include realigning (e.g., reorienting) cellular structures that increase tissue circumference. Reducing total energy delivery may correspond to a lower overall temperature increase of the tissue, which may increases the safety profile of the treatment procedure as well as promote a faster and safer healing cascade.

In some variations, negative pressure may be applied to the tissue to ensure even contact between tissue and an electrode array during treatment. For example, negative pressure or suction may be applied by an expandable member to a tissue lumen (e.g., duodenum, duodenal tissue) to facilitate tissue apposition with an electrode array of the expandable member. Higher tissue apposition may further enable a reduction in total energy delivery and improved treatment outcomes.

In some variations, stretching the tissue by applying a radially outward force using the expandable member and/or application of negative pressure to the tissue from the expandable member may reduce a range of tissue thicknesses. For example, the expandable member may stretch tissue such that a ratio of manipulated (e.g., compressed/stretched/dilated) tissue thickness to unmanipulated tissue thickness is about 0.5. In some variations, the combination of tissue stretching and application of a tissue treatment as described herein may synergistically treat a tissue of a patient.

In some variations, a tissue treatment device as described herein may transition to an expanded configuration to dilate (e.g., stretch, extend) the tissue during a treatment procedure. In some variations, tissue may be treated within a predetermined range of dilation ratios. In some variations, a ratio of dilated to undilated mucosa tissue may be between about 0.40 and about 0.60, between about 0.45 and about 0.55, and about 0.50, including all ranges and sub-values in-between. In some variations, a ratio of dilated to undilated submucosa tissue may be between about 0.15 and about 0.35, between about 0.20 and about 0.30, and about 0.26, including all ranges and sub-values in-between. In some variations, a ratio of dilated duodenum diameter to undilated duodenum diameter may be between about 1.5 and about 2.3, between about 1.7 and about 2.1, and about 1.91, including all ranges and sub-values in-between. In some variations, a ratio of a dilated duodenum diameter to an undilated duodenum diameter may be between about 1.5 and about 2.3, between about 1.7 and about 2.1, and about 1.91, including all ranges and sub-values in-between.

In some variations, a tissue treatment device may be configured to simultaneously dilate and suction tissue to the ablation device. In some variations, a ratio of suction and dilated to undilated mucosa tissue may be between about 0.40 and about 0.60, between about 0.45 and about 0.55, and about 0.47, including all ranges and sub-values in-between. In some variations, a ratio of suction and dilated to undilated submucosa tissue may be between about 0.20 and about 0.50, between about 0.30 and about 0.40, and about 0.33, including all ranges and sub-values in-between.

In some variations, the suction may be generated by the device itself while in the expanded configuration. Additionally or alternatively, the suction may be generated by a visualization device such as an endoscope. An amount of suction may be configured to secure uniform apposition of tissue to the surface of the expandable member (e.g., electrode surfaces). However, the amount of suction should not exceed a predetermined threshold corresponding to pressure necrosis. In some variations, the negative pressure (e.g., suction) applied to the tissue may be between about 50 mmHg and about 75 mmHg for less than about one minute. In some variations, the negative pressure (e.g., suction) applied to the tissue may be between about 10 mmHg and about 200 mmHg. The amount of suction may be a function of one or more of total surface area of the expandable member, number and size of the openings, time that suction is applied, edge condition of the openings, compliance of tissue, vascularization of tissue, and friability of tissue.

In some variations, an amount of tissue compliance may correspond to an amount of dilation and suction needed to ensure uniform surface contact of the electrodes and the desired tissue treatment. In some variations, the tissue may respond better to less dilation and more suction (or vice versa) depending on compliance and structure. In some variations, apposition may be assessed visually and/or through impedance measurement. In some variations, apposition may be measured using one or more temperature sensors, pressure sensors, and proximity sensors.

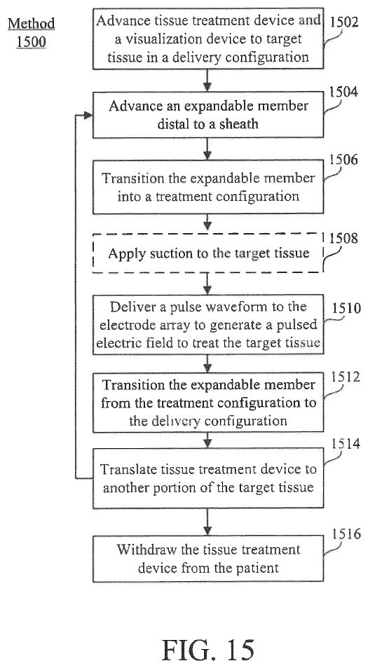

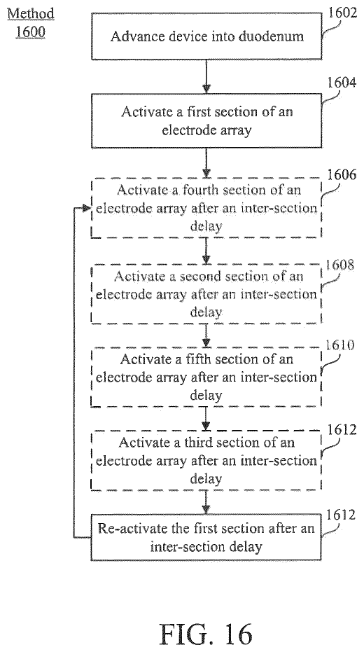

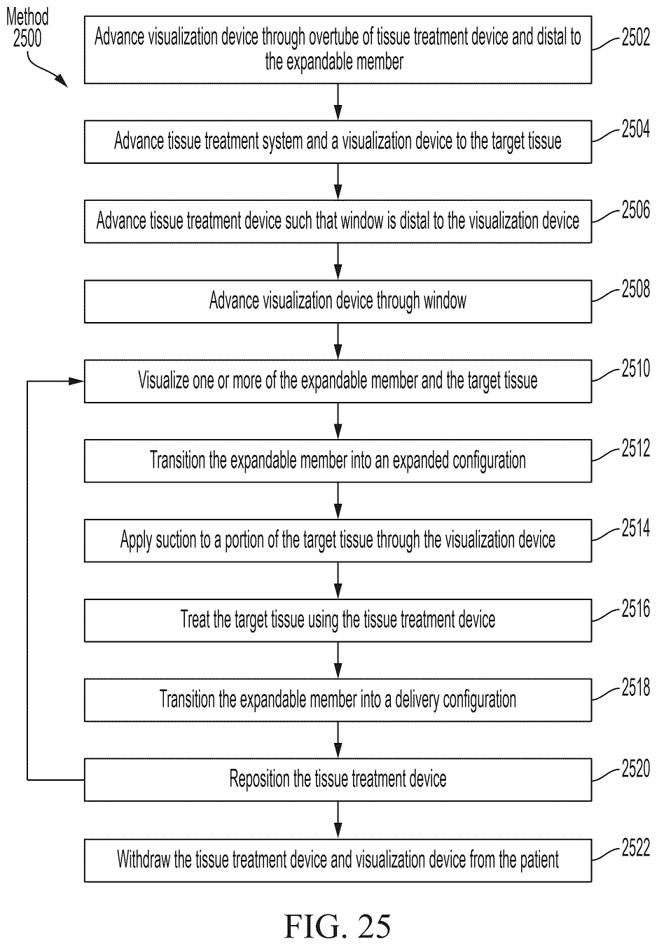

FIGS. 15 and 25 are flowcharts that generally describes variations of a method of treating a chronic condition (1500, 2500). In some of these variations, a patient may be positioned on their left lateral side or in a prone position during the procedure, and the target tissue may optionally be insufflated (e.g., using CO₂ or saline). The target tissue may include one or more of an esophagus, a stomach, a large intestine (e.g., cecum, colon, rectum, anal canal), a small intestine, any portion of the gastrointestinal tract, vasculature (e.g., blood vessels), a thoracic cavity (e.g., lungs), an abdomino-pelvic cavity, a pelvic cavity (e.g., bladder), a vertebral cavity, a cranial cavity (e.g., nasal passageway), and the like. For ease of explanation, the target tissue discussed with respect to FIGS. 15 and 25 correspond to a duodenum. In some variations, the methods (1500, 25) may be performed absent fluoroscopic guidance since the visualization device provides guidance confirmation and the tissue treatment device is coupled to the visualization device. For example, the tissue treatment system corresponding to FIG. 15 may comprise a tissue treatment device comprising an elongate body and an expandable member including an electrode array, and a sheath. In a delivery configuration, the expandable member may be disposed in the sheath circumferentially about the visualization device in an unexpanded configuration. The target tissue may comprise one or more of a duodenum, a pylorus, an esophagus, a stomach, a small intestine, a large intestine, a vasculature, a thoracic cavity, an abdomino-pelvic cavity, a pelvic cavity, a vertebral cavity, and a cranial cavity. In some variations, a size (e.g., diameter) of the target tissue of the patient to be treated may be estimated using a sizing device such as a pressure sensor configured to measure a pressure corresponding to a size of the target tissue. Treating the target tissue may treat one or more of Barrett's esophagus and a metabolic disorder comprising one or more of obesity, Non-alcoholic fatty liver disease (NAFLD), Nonalcoholic steatohepatitis (NASH), proinflammatory processes, immunological processes, Alzheimer's disease, neurological disorders, Type I diabetes, and Type II diabetes, and cancer.

The method (1500) may include advancing a tissue treatment device and a visualization device to a target tissue of a patient (1502). For example, a visualization device coupled to the tissue treatment device (e.g., pulsed electric field device) may be advanced concurrently over a guidewire into a target tissue (e.g., duodenum). For example, the guidewire, the tissue treatment device (e.g., pulsed electric field device), and the visualization device (e.g., endoscope) may be advanced through the pylorus, bulb of the duodenum, descending part, ampulla of V ater, duodenojejunal flexure, and up to and/or beyond the Ligament of Treitz. Then, while maintaining the position of the guidewire, the visualization device coupled to the tissue treatment device may be withdrawn proximal to a target tissue treatment site. The tissue treatment device may be advanced distal to the visualization device and disposed proximate to the target tissue to be treated. In some variations, a treatment site within the target tissue may be one or more of proximal and distal to the ampulla of Vater. For example, the expandable member of the tissue treatment device may be advanced about 1 cm to about 4 cm distal to the ampulla of Vater. Additionally or alternatively, the target tissue may be tissue corresponding to a bulb of the duodenum and/or the ampulla of V ater.

In some variations, the tissue treatment device and the visualization device may correspond to any of the tissue treatment devices (e.g., device (410)) and visualization devices described herein. For example, the tissue treatment device may comprise an elongate body and an expandable member coupled to the elongate body. The expandable member may comprise an electrode array, a first portion, and a second portion. A sheath may be configured to at least partially receive a visualization device and the expandable member. In some variations, the first portion (e.g., first lateral portion) may at least partially overlap with the second portion (e.g., second lateral portion) in the delivery configuration. For example, at least partially overlapping the first portion with the second portion in the delivery configuration may include positioning the first portion circumferentially about the visualization device in a first direction and positioning the second portion circumferentially about the visualization device in a second, opposite direction.

In step 1504, the expandable member of the tissue treatment device may advance distal to the sheath. For example, advancing the expandable member distal to the sheath may include translating the elongate member relative to the sheath. In some variations, an actuator coupled to the elongate body may be actuated to advance (e.g., push) the expandable member distal to a distal end of the sheath and visualization device. In some variations, the expandable member may be visualized using the visualization device positioned within the sheath.

Additionally or alternatively, the tissue treatment device may comprise a second expandable member (e.g., inflatable member, balloon) comprising a second electrode array disposed distal to a first expandable member. In some of these variations, the second expandable member may be advanced, inflated, and energized in the same manner as the first expandable member

In step 1506, the expandable member of the tissue treatment device may transition from a delivery configuration to a treatment (e.g., inflated, expanded) configuration. In some variations, the expandable member in the treatment configuration may dilate a portion of the target tissue in contact with the expandable member, which may be visualized by the visualization device. In some variations, the expandable member may comprise a balloon that may be inflated via an inflation lumen of the elongate body and/or suction catheter. In some variations, the expandable member may be inflated to a first diameter where at least one pleat of the expandable member is in a folded configuration. Further inflation of the expandable member may transition the pleat to an unfolded configuration where the expandable member has a second diameter larger than the first diameter. In some variations, a diameter of the expandable member in the treatment configuration may be based on an estimated size of the target tissue.

In step 1508, suction may be applied to a portion of the target tissue through one or more fluid openings of the electrode array of the expandable member. For example, a suction catheter may be advanced from a lumen of the visualization device near an expandable member. In some variations, the visualization device may be positioned proximally of the expandable member as the suction catheter is advanced. A negative pressure source coupled to the suction catheter may be configured to generate a negative pressure (e.g., suction) that suctions tissue to a surface of the electrode array. The close contact between the tissue and the expandable member may improve energy delivery and treatment outcomes. One or more pulse waveforms may be delivered while suction is being applied. In some variations, suction may be applied during delivery of a pulse waveform and reduced during time periods when tissue treatment energy is not delivered. For example, suction may be reduced (e.g., halted) during a time period after energy delivery, and when one or more of the tissue treatment device and visualization device are advanced within the duodenum. Thus, suction may be generated intermittently throughout a treatment procedure (e.g., concurrent with energy delivery). An amount of suction applied to one or more portions of tissue may be as described herein.

In step 1510, one or more pulse waveforms may be delivered to an electrode array of an expandable member to generate a pulsed or modulated electric field. In some variations, the electrode array may have a plurality of sections arranged circumferentially about the first elongate body. For example, the electrode array may have two, three, four, or five sections exposed to tissue when in the expanded configuration. The operator may confirm tissue contact with a predetermined number of electrode sections and may select the corresponding electrode sections for energy delivery from a signal generator. In some variations, one or more of the electrode sections may be separated by a pleat such that an unfolded pleat will increase a distance between the electrode sections and increase a diameter of the expandable member while a folded pleat will conversely decrease a distance between the electrode sections and decrease a diameter of the expandable member.

In some variations, the signal generator may be configured to deliver a tissue treatment waveform to two or more non-proximate sections of the plurality of sections in a predetermined sequence. For example, the signal generator may generate a waveform sequence (e.g., interleaving waveform) having an inter-section delay between sections of an electrode array. In particular, the predetermined sequence may comprise an inter-section delay between delivery of a first pulsed electric field waveform to a first section of the plurality of sections and a second pulsed electric field waveform to a second section of the plurality of sections. The first and second pulsed electric field waveforms may be the same or different. In some variations, the inter-section delay may be between about 10 ms and about 4000 ms. In some variations, the first and second sections are non-adjacent (e.g., not immediately next to each other) sections. In some variations, the predetermined sequence may further comprise an intra-section delay between delivery of the first pulsed electric field waveform to the first section and delivery of a second pulsed electric field waveform to the first section.

In some variations, the intra-section delay may be between about 1 seconds and about 10 seconds. In some variations, the first and second pulsed electric field waveforms may comprise a series of between about 10 bipolar pulses and about 500 bipolar pulses. In some variations, each of the bipolar pulses may comprise a pulse width between about 1 μs and about 3 μs.

In some variations, the first and second pulsed electric field waveforms may comprise the same number of bipolar pulses. In some variations, the first and second pulsed electric field waveforms may comprise a different number of bipolar pulses. In some variations, between about 0.05 J per bipolar pulse and about 0.5 J per bipolar pulse may be delivered to the electrode array. In some variations, an instantaneous power between about 26,000 W per bipolar pulse and about 70,000 W per bipolar pulse may be delivered by the electrode array. In some variations, the predetermined sequence may be repeated between about 5 and about 15 times. In some variations, activation of the plurality of sections may have a cumulative activation time between about 0.1 ms and about 10 ms over a treatment time between about 30 seconds and about 35 seconds. In some variations, the predetermined sequence may comprise a duty cycle between about 0.003% and about 0.004%. In some variations, the plurality of sections may comprise between abut one section and about ten sections, between about two sections and eight sections, between about three sections and seven sections, and up to five sections, including all ranges and sub-values in-between. In some variations, the electrode array may comprise a surface area between about 4 square centimeters and about 42 square centimeters. In some variations, each section of the plurality of sections may comprise a plurality of electrodes. In some variations, each section of the plurality of sections may comprise between 10 and 18 electrodes.

In some variations, a pulsed electric field waveform (e.g., interleaving waveform) may be delivered in a predetermined sequence to each of a first section and a second section non-proximate to the first section. The predetermined sequence may have an inter-section delay between the first and second sections of an electrode array.

In some variations, the intra-section delay may be between about 10 ms and about 10,000 ms, between about 5000 ms and about 10,000 ms, between about 10 ms and about 5000 ms, and between about 2000 ms and about 8000 ms, including all ranges and sub-values in-between. In some variations, the first section may be re-activated after an intra-section delay relative to a previous activation of the first section. For example, the intra-section delay may be between about 3 seconds and about 5 seconds. In some variations, the pulsed electric field waveform may comprise a series of between about 40 bipolar pulses and about 60 bipolar pulses. In some variations, each of the bipolar pulses may comprise a pulse width between about 1 μs and about 3 μs. In some variations, activating each of the first and second sections may deliver between about 0.05 J per bipolar pulse and about 0.5 J per bipolar pulse. In some variations, activating each of the first and second sections may deliver an instantaneous power between about 38,800 W per bipolar pulse and about 41,250 W per bipolar pulse. In some variations, each of the bipolar pulses may comprise a positively-charged portion and a negatively-charged portion each having a pulse width between about 1.3 μs and about 1.5 μs. In some variations, each of the bipolar pulses may comprise a time interval between the positively-charged and negatively-charged portions. In some variations, the time interval may be between about 0.05 μs and about 0.1 μs.

In some variations, the first and second sections are non-proximate. In some variations, the electrode array may further comprise one or more of a third section, a fourth section, and a fifth section. In some variations, the predetermined sequence may further comprise activating the one or more of the third section, fourth section, and fifth section with the inter-section delay between activation of successive sections. In some variations, the first and second sections may be activated for a cumulative activation time between about 0.1 ms and about 10 ms over a treatment time between about 30 and about 35 seconds. In some variations, the first wherein the predetermined sequence comprises a duty cycle between about 0.003% and about 0.004%. In some variations, the predetermined sequence may be repeated between about 5 and about 15 times.

The characteristics associated with the pulse waveform may correspond to an amount of energy generated by the electrode array, which in turn may be applied to tissue. The amount of energy may correspond to one or more electric fields generated by the electrode array.

In some variations, the same portion of tissue may be treated multiple times (e.g., double treated). Treating a same portion of tissue a plurality of times (e.g., two times, three times, four times) may increase the percentage of the tissue in the portion having been treated, thus yielding a more complete lesion leading to improved outcomes. The same pulse waveform energy parameters as first delivered in step 1510 or different pulse waveform energy parameters may be delivered to the same portion of tissue (e.g., gastrointestinal tract, including but not limited to, the duodenum, pylorus, esophagus, stomach, small intestine, and large intestine) when treating the same portion of tissue a plurality of times. In some variations, the pulsed waveform comprises a first pulsed waveform, and delivering at least a second pulsed waveform to the electrode array to generate a second pulsed or modulated electric field thereby treating at least a portion of the tissue previously treated. A plurality of treatments at the same portion of tissue improves the homogeneity of the treatment rather than a depth of penetration. In some variations where the same portion of tissue is treated multiple times, the expandable member may be rotated by a predetermined angle to ensure circumferential coverage where the electrode array does not extend across an entire circumference of the expandable member.

In some variations, the method may include measuring a temperature of the tissue during treatment using a temperature sensor as described herein, and the measured temperature may be between about 37° C. and about 45° C. (e.g., an increase of between about 3° C. and 8° C.) during delivery of the pulsed waveform. Put another way, delivery of the pulsed or modulated electric field created by the pulsed waveforms described herein may produce an increase in tissue temperature of between about 3° C. and 8° C. and a resultant tissue temperature of between about 37° C. and about 45° C. For example, a target temperature achieved by application of the pulsed or modulated electric fields created by the pulsed waveforms described herein may be at about 41° C., which may correspond to about a 4° C. to about 5° C. temperature increase in the tissue. In some variations, the method may include increasing a temperature of the tissue to about 41° C. before delivering the pulsed waveform.

In some variations, as described in more detail herein, tissue may be compressed during treatment with the pulsed or modulated electric field. In these variations, the pulsed or modulated electric field may be a therapeutic electric field that treats tissue at a compressed tissue depth of between about 0.25 mm and about 0.75 mm and at an uncompressed tissue depth of between about 0.50 mm and about 1.5 mm.

In step 1512, the expandable member (1452) may be transitioned from the treatment configuration to the delivery configuration (or a flattened configuration). This allows the tissue treatment device and the visualization device to be slidably translated together relative to the duodenum. In some variations, the suction catheter may be used to remove residual fluids and/or improve visualization. In some variations, once energy delivery is completed to the portion of tissue, the suction catheter may be withdrawn (e.g., retracted) into the lumen of the visualization device (1440). The treated tissue may be inspected for signs of thermal or physical injury.

In some variations, the expandable member may transition to the unexpanded configuration (e.g., become deflated) and repositioned and/or withdrawn from the patient body. Additionally or alternatively, the expandable member may be retracted into the sheath to reposition the system to the delivery configuration. In this manner, the tissue treatment device may be repositioned to treat a second portion of target tissue. For example, the treatment device may be repositioned proximally to treat the second target tissue before retracting the expandable member to reposition the system to the delivery configuration. In some variations, the tissue treatment device may be repositioned proximal or distal to the previously treated target tissue. For example, treatment may begin at a distal end of the duodenum with sequential treatment of proximal portions of the duodenum.

In step 1514, the tissue treatment device may be translated (e.g., advanced) to another portion of tissue to be treated where steps 1504-1512 may be repeated as desired. For example, the tissue treatment device and/or visualization device may be advanced through the duodenum multiple times to repeat the energy delivery process described herein. In some variations, the proximal edge of the expandable member (e.g., electrode array) may be aligned against an edge of the treated tissue. In some variations, the expandable member may be translated to another portion of tissue without deflating the expandable member and transitioning the tissue treatment device into a delivery configuration.

In some variations, the duodenum may be treated over about 2 portions to about 20 portions, about 6 portions to about 15 portions, about 6 portions to about 10 portions, about 10 portions to about 12 portions, including all ranges and sub-values in-between. In some variations, a total treatment length of tissue may be between about 6 cm and about 20 cm. In some variations, a portion of the tissue may have a circumference between about 22 mm and an average of about 25 mm. In some variations, more than about 60 percent of a circumference of a portion of the duodenum may be treated.

In step 1516, the tissue treatment device and the visualization device may be withdrawn from the patient. The tissue treatment device and the visualization device may be withdrawn from the patient simultaneously or sequentially. For example, the tissue treatment device and visualization device may be withdrawn from the patient, and the visualization may be reintroduced into the patient to inspect the treated tissue.

The method (2500) of FIG. 25 may include advancing a visualization device through the lumen of the overtube and distal to an expandable member at step 2502. As shown in FIGS. 20A and 20B, a visualization device (2040) may be advanced through a lumen of overtube (2010) such that a distal end of the visualization device (2040) may be distal to a distal end of the expandable member (2020). As shown in FIGS. 20A-20E, the tissue treatment system may comprise a tissue treatment device (2000) comprising an overtube (2010) defining a lumen and a window (2012), and an expandable member (2020) coupled to the overtube.

In step 2504, a tissue treatment system and a visualization device may be advanced to the target tissue of a patient. For example, the tissue treatment device (e.g., pulsed electric field device) and the visualization device (e.g., endoscope) may be advanced through the pylorus, bulb of the duodenum, descending part, ampulla of V ater, duodenojejunal flexure, and up to and/or beyond the Ligament of Treitz. Furthermore, translation of the visualization device at a handle may simultaneously translate the tissue treatment device riding over the visualization device through one or more body cavities. In some variations, the tissue treatment system and the visualization device may be advanced to the target tissue of a patient in a delivery configuration where the expandable member is in an unexpanded configuration.

In step 2506, the tissue treatment device may be advanced relative to the visualization device such that the window is distal to the visualization device (i.e., distal to a distal tip of the visualization device). For example, the tissue treatment device may be advanced distal to the visualization device and disposed proximate to the target tissue to be treated. In some variations, a treatment site within the target tissue may be one or more of proximal and distal to the ampulla of Vater. For example, the expandable member of the tissue treatment device may be advanced about 1 cm to about 4 cm distal to the ampulla of Vater. Additionally or alternatively, the target tissue may be tissue corresponding to a bulb of the duodenum and/or the ampulla of V ater.

In step 2508, the visualization device may be advanced through the window of the overtube. As shown in FIGS. 20D and 20E, the visualization device (2040) advanced through the window (2012) may be proximal to the treatment member (e.g., electrode array (2030)) of expandable member (2020). Furthermore, a distal end of the visualization device may be disposed external to the overtube and the expandable member while a portion of the visualization device is disposed within a lumen of the overtube.

In step 2510, one or more of the expandable member and the target tissue may be visualized after the visualization device is advanced through the window of the overtube. For example, tissue anatomy (e.g., an ampulla of the duodenum) may be identified by visualizing the target tissue using the visualization device. The expandable member may be repositioned as desired. For example, the expandable member may be positioned a predetermined distance distal to the ampulla.

In step 2512, the expandable member of the tissue treatment device may transition from a delivery configuration to a treatment (e.g., inflated, expanded) configuration. In some variations, the expandable member in the treatment configuration may dilate a portion of the target tissue in contact with the expandable member, which may be visualized by the visualization device. In some variations, the expandable member may comprise a balloon that may be inflated via an inflation lumen of the overtube. In some variations, the expandable member may be inflated to a first diameter where at least one pleat of the expandable member is in a folded configuration. Further inflation of the expandable member may transition the pleat to an unfolded configuration where the expandable member has a second diameter larger than the first diameter. In some variations, a diameter of the expandable member in the treatment configuration may be based on an estimated size of the target tissue. A spacing of electrodes of the electrode array may be maintained when the expandable member is transitioned to the first and second diameters such that the therapeutic electric field generated by the electrode array may be constant for different diameters.

In step 2514, suction may be applied to a portion of the target tissue through one or more of the visualization device and the lumen of the overtube. For example, a lumen (e.g., working channel) of an endoscope advanced from a window of an overtube may be positioned adjacent an expandable member to apply suction to improve apposition between tissue and the expandable member. In some variations, the visualization device may be positioned proximally of the expandable member as the suction is applied. A negative pressure source coupled to the visualization device may be configured to generate a negative pressure (e.g., suction) that suctions tissue to a surface of the treatment member and/or expandable member from a proximal end and a distal end of the expandable member. For example, suction applied through a working channel of a visualization device may generate a negative pressure through the window of the overtube and the lumen of the expandable member such that negative pressure may be applied to tissue and the expandable member at a distal end of the tissue treatment device. Additionally or alternatively, the overtube may comprise an inflation lumen. The close contact between the tissue and the expandable member may improve energy delivery and treatment outcomes. One or more pulse waveforms may be delivered while suction is being applied. In some variations, suction may be applied during delivery of a pulse waveform and reduced during time periods when tissue treatment energy is not delivered. For example, suction may be reduced (e.g., halted) during a time period after energy delivery, and when one or more of the tissue treatment device and visualization device are advanced within a body cavity (e.g., duodenum, esophagus). Thus, suction may be generated intermittently throughout a treatment procedure (e.g., concurrent with energy delivery). An amount of suction applied to one or more portions of tissue may be as described herein.

In step 2516, target tissue may be treated using the expandable member. For example, one or more pulse waveforms may be delivered to an electrode array of an expandable member to generate a pulsed or modulated electric field. Additionally or alternatively, one or more of thermal energy (e.g., heat-based ablation, cryogenic fluid), pulsed-electric field energy, ultrasonic energy (e.g., piezoelectric transducer), vapor energy, radiofrequency energy, laser energy, and mechanical energy (e.g., blade) may be applied to treat tissue. In some variations, the electrode array may have a plurality of sections arranged circumferentially about the expandable member. For example, the electrode array may have two, three, four, or five sections exposed to tissue when in the expanded configuration. The operator may confirm tissue contact with a predetermined number of electrode sections and may select the corresponding electrode sections for energy delivery from a signal generator. In some variations, one or more of the electrode sections may be separated by a pleat such that an unfolded pleat will increase a distance between the electrode sections and increase a diameter of the expandable member while a folded pleat will conversely decrease a distance between the electrode sections and decrease a diameter of the expandable member.

In some variations, the signal generator may be configured to deliver a tissue treatment waveform to two or more non-proximate sections of the plurality of sections in a predetermined sequence. For example, the signal generator may generate a waveform sequence (e.g., interleaving waveform) having an inter-section delay between sections of an electrode array. In particular, the predetermined sequence may comprise an inter-section delay between delivery of a first pulsed electric field waveform to a first section of the plurality of sections and a second pulsed electric field waveform to a second section of the plurality of sections. The first and second pulsed electric field waveforms may be the same or different. In some variations, the inter-section delay may be between about 10 ms and about 4000 ms. In some variations, the first and second sections are non-adjacent (e.g., not immediately next to each other) sections. In some variations, the predetermined sequence may further comprise an intra-section delay between delivery of the first pulsed electric field waveform to the first section and delivery of a second pulsed electric field waveform to the first section.

In some variations, the intra-section delay may be between about 1 seconds and about 10 seconds. In some variations, the first and second pulsed electric field waveforms may comprise a series of between about 10 bipolar pulses and about 500 bipolar pulses. In some variations, each of the bipolar pulses may comprise a pulse width between about 1 μs and about 3 μs.

In some variations, the first and second pulsed electric field waveforms may comprise the same number of bipolar pulses. In some variations, the first and second pulsed electric field waveforms may comprise a different number of bipolar pulses. In some variations, between about 0.05 J per bipolar pulse and about 0.5 J per bipolar pulse may be delivered to the electrode array. In some variations, an instantaneous power between about 26,000 W per bipolar pulse and about 70,000 W per bipolar pulse may be delivered by the electrode array. In some variations, the predetermined sequence may be repeated between about 5 and about 15 times. In some variations, activation of the plurality of sections may have a cumulative activation time between about 0.1 ms and about 10 ms over a treatment time between about 30 seconds and about 35 seconds. In some variations, the predetermined sequence may comprise a duty cycle between about 0.003% and about 0.004%. In some variations, the plurality of sections may comprise between abut one section and about ten sections, between about two sections and eight sections, between about three sections and seven sections, and up to five sections, including all ranges and sub-values in-between. In some variations, the electrode array may comprise a surface area between about 4 square centimeters and about 42 square centimeters. In some variations, each section of the plurality of sections may comprise a plurality of electrodes. In some variations, each section of the plurality of sections may comprise between 10 and 18 electrodes.

In some variations, a pulsed electric field waveform (e.g., interleaving waveform) may be delivered in a predetermined sequence to each of a first section and a second section non-proximate to the first section. The predetermined sequence may have an inter-section delay between the first and second sections of an electrode array.

In some variations, the intra-section delay may be between about 10 ms and about 10,000 ms, between about 5000 ms and about 10,000 ms, between about 10 ms and about 5000 ms, and between about 2000 ms and about 8000 ms, including all ranges and sub-values in-between. In some variations, the first section may be re-activated after an intra-section delay relative to a previous activation of the first section. For example, the intra-section delay may be between about 3 seconds and about 5 seconds. In some variations, the pulsed electric field waveform may comprise a series of between about 40 bipolar pulses and about 60 bipolar pulses. In some variations, each of the bipolar pulses may comprise a pulse width between about 1 μs and about 3 μs. In some variations, activating each of the first and second sections may deliver between about 0.05 J per bipolar pulse and about 0.5 J per bipolar pulse. In some variations, activating each of the first and second sections may deliver an instantaneous power between about 38,800 W per bipolar pulse and about 41,250 W per bipolar pulse. In some variations, each of the bipolar pulses may comprise a positively-charged portion and a negatively-charged portion each having a pulse width between about 1.3 μs and about 1.5 μs. In some variations, each of the bipolar pulses may comprise a time interval between the positively-charged and negatively-charged portions. In some variations, the time interval may be between about 0.05 μs and about 0.1 μs.

In some variations, the first and second sections are non-proximate. In some variations, the electrode array may further comprise one or more of a third section, a fourth section, and a fifth section. In some variations, the predetermined sequence may further comprise activating the one or more of the third section, fourth section, and fifth section with the inter-section delay between activation of successive sections. In some variations, the first and second sections may be activated for a cumulative activation time between about 0.1 ms and about 10 ms over a treatment time between about 30 and about 35 seconds. In some variations, the first wherein the predetermined sequence comprises a duty cycle between about 0.003% and about 0.004%. In some variations, the predetermined sequence may be repeated between about 5 and about 15 times.

The characteristics associated with the pulse waveform may correspond to an amount of energy generated by the electrode array, which in turn may be applied to tissue. The amount of energy may correspond to one or more electric fields generated by the electrode array.

In some variations, the target tissue may be re-treated one or more times using the expandable member. For example, the same portion of tissue may be treated multiple times (e.g., double treated, triple treated). Treating a same portion of tissue a plurality of times (e.g., two times, three times, four times) may increase the percentage of the tissue in the portion having been treated, thus yielding a more complete lesion leading to improved outcomes. The same pulse waveform energy parameters as first delivered in step 2516 or different pulse waveform energy parameters may be delivered to the same portion of tissue (e.g., gastrointestinal tract, including but not limited to, the duodenum, pylorus, esophagus, stomach, small intestine, and large intestine) when treating the same portion of tissue a plurality of times. In some variations, the pulsed waveform comprises a first pulsed waveform, and delivering at least a second pulsed waveform to the electrode array to generate a second pulsed or modulated electric field thereby treating at least a portion of the tissue previously treated. A plurality of treatments at the same portion of tissue improves the homogeneity of the treatment rather than a depth of penetration. In some variations where the same portion of tissue is treated multiple times, the expandable member may be rotated by a predetermined angle (e.g., about 1 degree to about 360 degrees) to ensure circumferential coverage where the electrode array does not extend across an entire circumference of the expandable member. Furthermore, the treated target tissue may be visualized before re-treating the target tissue and before treating another target tissue.

In step 2518, the expandable member (1452) may optionally be transitioned from the treatment configuration to the delivery configuration (or a flattened configuration) to facilitate movement of the tissue treatment device. This allows the tissue treatment device and the visualization device to be slidably translated together relative to the treatment site (e.g., duodenum). In some variations, the suction may be applied to remove residual fluids and/or improve visualization. The treated tissue may be inspected for signs of thermal or physical injury.

In step 2520, the tissue treatment device may be repositioned (e.g., translated, rotated) to treat another portion of tissue where steps 2510-2518 may be repeated as desired. For example, the tissue treatment device and/or visualization device may be advanced through the duodenum multiple times to repeat the energy delivery process described herein. In some variations, the treatment device may be repositioned proximally or distally of the target tissue to treat a second portion of the target tissue or the treatment device may be rotatably repositioned to treat the second portion of the target tissue. In some variations, the proximal edge of the expandable member (e.g., electrode array) may be aligned against an edge of the treated tissue. In some variations, the expandable member may be rotated and/or translated to another portion of tissue without deflating the expandable member and transitioning the tissue treatment device into a delivery configuration.

In some variations, the target tissue (e.g., duodenum, esophagus) may be treated over about 2 portions to about 20 portions, about 6 portions to about 15 portions, about 6 portions to about 10 portions, about 10 portions to about 12 portions, including all ranges and sub-values in-between. In some variations, a total treatment length of tissue may be between about 6 cm and about 20 cm. In some variations, a portion of the tissue may have a circumference between about 22 mm and an average of about 25 mm. In some variations, a portion of a circumference of a portion of the duodenum may be treated including more than about 60 percent, more than about 70 percent, more than about 80 percent, more than about 90 percent, about 60 percent to about 100 percent, about 60 percent to about 90 percent, about 60 percent to about 80 percent, about 60 percent to about 70 percent, about 70 percent to about 100 percent, about 80 percent to about 100 percent, about 70 percent to about 90 percent, about 60 percent, about 70 percent, about 80 percent, about 90 percent, about 100 percent, including all ranges and sub-values in-between.

In some variations, the method may include measuring a temperature of the tissue during treatment using a temperature sensor as described herein, and the measured temperature may be between about 37° C. and about 45° C. (e.g., an increase of between about 3° C. and 8° C.) during delivery of the pulsed waveform. Put another way, delivery of the pulsed or modulated electric field created by the pulsed waveforms described herein may produce an increase in tissue temperature of between about 3° C. and 8° C. and a resultant tissue temperature of between about 37° C. and about 45° C. For example, a target temperature achieved by application of the pulsed or modulated electric fields created by the pulsed waveforms described herein may be at about 41° C., which may correspond to about a 4° C. to about 5° C. temperature increase in the tissue. In some variations, the method may include increasing a temperature of the tissue to about 41° C. before delivering the pulsed waveform.

In some variations, as described in more detail herein, tissue may be compressed during treatment. In these variations, tissue may be treated at a compressed tissue depth of between about 0.25 mm and about 0.75 mm and at an uncompressed tissue depth of between about 0.50 mm and about 1.5 mm.

In step 2522, the tissue treatment device and the visualization device may be withdrawn from the patient. The tissue treatment device and the visualization device may be withdrawn from the patient simultaneously or sequentially. For example, the tissue treatment device and visualization device may be withdrawn from the patient, and the visualization may be reintroduced into the patient to inspect the treated tissue.

### Method of Manufacturing

Generally, methods of manufacturing a treatment device may include coupling (e.g., attaching, bonding) a treatment member (e.g., electrode array) to a surface of the expandable member such that the electrode array may maintain its original geometric characteristics (e.g., size, shape, electrode spacing) even when the expandable member itself transitions between an unexpanded configuration and an expanded configuration. In some variations, the manufactured treatment device may include an elongate body (e.g., overtube), an expandable member (e.g., balloon) coupled to the elongate body, and treatment member (e.g., electrode array) coupled to the expandable member. The electrode array may comprise a substrate defining a plurality of apertures. A bonding layer may be bonded to the electrode array and the expandable member using the apertures of the substrate. The expandable member may comprise one or more of a thermoplastic urethane, thermoplastic elastomer, polyethylene terephthalate, polyimide, nylon, and biaxially-oriented polyethylene terephthalate.

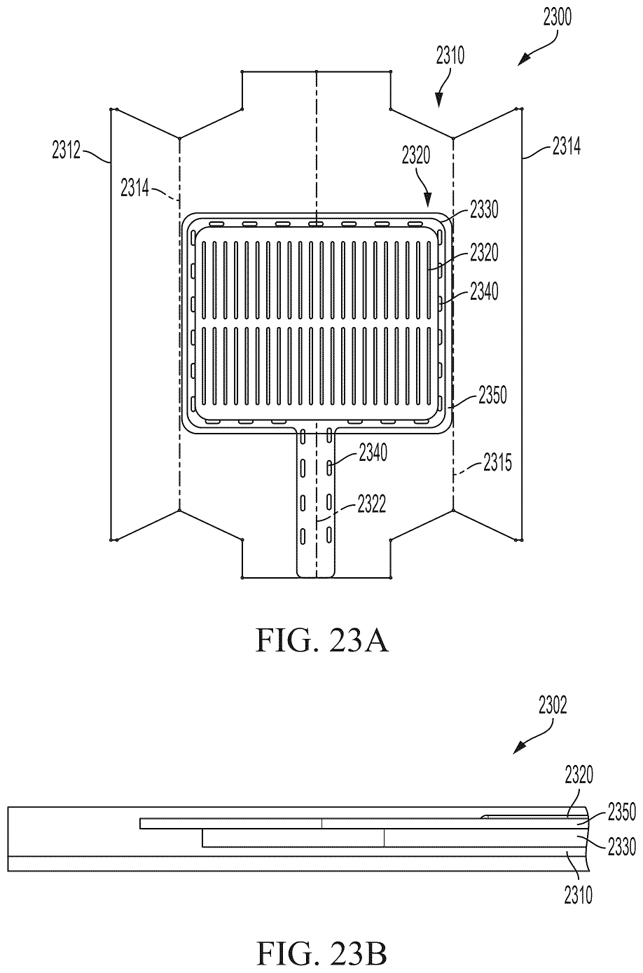

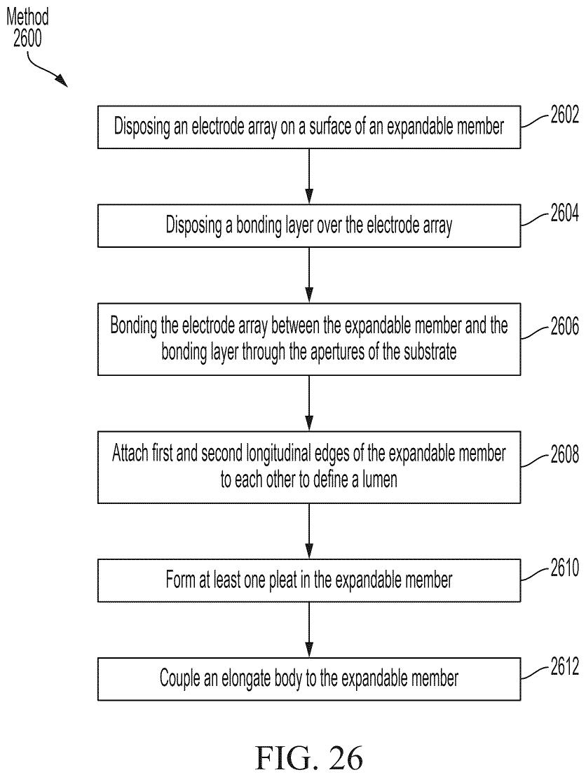

FIG. 26 is a flowchart that generally describes a variation of a method of manufacturing a tissue treatment device (2600). The method (2600) may include disposing a treatment member in the form of an electrode array on a surface of an expandable member (2602). As shown in the plan view schematic diagram (2300) of FIG. 23A, an electrode array (2320) is disposed on an unassembled (e.g., laser-cut patterned) expandable member (2310). The electrode array (2320) may comprise a plurality of spaced-apart electrodes (2320), a lead wire (2322), and a substrate (2330) (e.g., flex circuit). The substrate (2330) may define one or more apertures (2340) along an outer perimeter of the substrate (2330). In some variations, the apertures (2340) may have a rounded shape (e.g., circular, oval, ellipse) to reduce stress. The apertures (2340) may be spaced apart from each other by about 0.02 inches to about 0.03 inches, about 0.02 inches to about 0.025 inches, about 0.025 inches to about 0.03 inches, about 0.02 inches, about 0.025 inches, or about 0.03 inches, including all ranges and sub-values therebetween. The apertures (2340) may have a diameter of about 0.0005 inches to about 0.0015 inches, about 0.0005 inches to about 0.0010 inches, about 0.0010 inches to about 0.0015 inches, about 0.0005 inches, about 0.0010 inches, or about 0.0015 inches, including all ranges and sub-values therebetween.

In step 2604, a bonding layer may be disposed over the electrode array. For example, FIG. 23B depicts a cross-sectional schematic view (2302) of the treatment device where the bonding layer (2350) covers a perimeter of the electrode array (2320) such that the substrate (2330) is sandwiched between the bonding layer (2350) and the expandable member (2310). In particular, the bonding layer (2350) may cover the apertures of the substrate (2330).

In step 2606, the electrode array may be bonded between the expandable member and the bonding layer using the apertures of the substrate. For example, the electrode array (2320) may be bonded to the expandable member (2310) and the bonding layer (2350) using one or more of heat, pressure, an adhesive, and a chemical. For example, a press having a heating element may be used to apply a predetermined amount of pressure and heat to the bonding layer (2350), electrode array (2320), and expandable member (2310) to bond them together using (e.g., through) the apertures (2340) of the substrate (2330).

In step 2608, a first longitudinal edge of the expandable member may be attached to a second longitudinal edge of the expandable member to define a lumen of the expandable member. For example, a first longitudinal edge (2312) of the expandable member (2310) may comprise a first portion of a pleat, and a second longitudinal edge (2314) of the expandable member (2310) may comprise a second portion of the pleat. In step 2610, at least one pleat may be formed in the expandable member. For example, the attached longitudinal edges (2312, 2314) may be folded inward to form seams along edges (2313, 2315).

In step 2612, a distal end of an elongate body (e.g., overtube) may be coupled to an inner surface of a proximal end of the expandable member. For example, the expandable member may attach to the elongate body through one or more of an interference fit, an adhesive, a chemical, heat and/or pressure treatment, combinations thereof, and the like.

It should be understood that the examples and illustrations in this disclosure serve exemplary purposes and departures and variations such as the number of electrodes and devices, and so on can be built and deployed according to the teachings herein without departing from the scope of this invention.

As used herein, the terms “about” and/or “approximately” when used in conjunction with numerical values and/or ranges generally refer to those numerical values and/or ranges near to a recited numerical value and/or range. In some instances, the terms “about” and “approximately” may mean within +10% of the recited value. For example, in some instances, “about 100 [units]” may mean within #10% of 100 (e.g., from 90 to 110). The terms “about” and “approximately” may be used interchangeably.

### EXEMPLARY EMBODIMENTS

The following describe certain exemplary embodiments according to the present disclosure.

Embodiment 1. A system for treating tissue, comprising:
an elongate body;
an expandable member coupled to the elongate body, the expandable member comprising an electrode array, a first portion, and a second portion; and
a sheath at least partially receiving a visualization device and the expandable member,
wherein the first portion is positioned circumferentially about the visualization device in a first direction and the second portion is positioned circumferentially about the visualization device in a second, opposite direction.

Embodiment 2. The system of embodiment 1, wherein the first lateral portion is at least partially overlapped by the second lateral portion.

Embodiment 3. The system of any of the preceding embodiments, wherein the sheath is attached to the visualization device.

Embodiment 4. The system of any of the preceding embodiments, wherein the elongate body is configured to translate the expandable member relative to the sheath.

Embodiment 5. The system of any of the preceding embodiments, wherein the elongate body comprises one or more of an inflation lumen, a suction lumen, a pull wire, and a lead wire.

Embodiment 6. The system of any of the preceding embodiments, wherein the electrode array is coupled to the expandable member via a thermal seal.

Embodiment 7. The system of any of the preceding embodiments, wherein the elongate body comprises a suction lumen, the suction lumen at least partially disposed between the electrode array and expandable member such that suction is applied to the target tissue through the electrode array.

Embodiment 8. The system of any of the preceding embodiments, wherein in an expanded configuration, the expandable member comprises a first arc length and the electrode array comprises a second arc length less than the first arc length.

Embodiment 9. The system of any of the preceding embodiments, wherein the expandable member comprises one or more of a thermoplastic urethane, thermoplastic elastomer, polyethylene terephthalate, polyimide, nylon, and biaxially-oriented polyethylene terephthalate.

Embodiment 10. The system of any of the preceding embodiments, wherein the expandable member is transparent.

Embodiment 11. The system of any of the preceding embodiments further comprising a handle including an actuator configured to translate the elongate body relative to the sheath.

Embodiment 12. The system of any of the preceding embodiments further comprising a fastener configured to couple the elongate body to visualization device.

Embodiment 13. The system of any of the preceding embodiments, wherein the expandable member comprises a plurality of expandable members arranged serially.

Embodiment 14. The system of any of the preceding embodiments further comprising one more of a pressure sensor, temperature senor, and proximity sensor.

Embodiment 15. The device of any of the preceding embodiments, wherein the expandable member comprises a support member.

Embodiment 16. A system for treating tissue, comprising:
an elongate body;
an expandable member coupled to the elongate body, the expandable member comprising an electrode array and defining a longitudinal axis, wherein the expandable member is asymmetric relative to the longitudinal axis; and
a sheath at least partially receiving a visualization device and the expandable member,
wherein in a delivery configuration the expandable member is disposed between an inner surface of the sheath and an outer surface of the visualization device.

Embodiment 17. The system of any of the preceding embodiments, wherein the expandable member is disposed distal to the sheath in a treatment configuration.

Embodiment 18. The system of any of the preceding embodiments further comprising an actuator coupled to the elongate body, the actuator configured to transition the elongate body and expandable member between the delivery configuration and the treatment configuration by translating the elongate body and expandable member relative to the sheath.

Embodiment 19. The system of any of the preceding embodiments, wherein the expandable member comprises a first asymmetric portion and a second asymmetric portion, wherein when transitioning from the treatment configuration to the delivery configuration the first asymmetric portion is configured to be between the second asymmetric portion and the endoscope.

Embodiment 20. The system of any of the preceding embodiments, wherein a combined diameter of the system in the delivery configuration is less than about 17 mm.

Embodiment 21. The system of any of the preceding embodiments, wherein the expandable member further comprises a proximal tapered portion.

Embodiment 22. The system of any of the preceding embodiments, wherein the proximal tapered portion and the longitudinal axis form an angle between about 10 degrees and about 80 degrees.

Embodiment 23. The system of any of the preceding embodiments, wherein the expandable member is eccentrically coupled to the elongate body such that a longitudinal axis of the elongate body does not align with the longitudinal axis of the expandable member.

Embodiment 24. The system of any of the preceding embodiments, wherein the elongate body is coupled to a sidewall of the expandable member.

Embodiment 25. The system of any of the preceding embodiments, wherein the proximal tapered portion comprises a first lateral taper and a second lateral taper asymmetric to the first lateral taper.

Embodiment 26. A system for treating tissue, comprising:
an elongate body;
a balloon coupled to the elongate body, the balloon comprising an electrode array, a first lateral portion, and a second lateral portion; and
a sheath at least partially housing a visualization device and the balloon,
wherein when housed in the sheath the first lateral portion is rolled around the visualization device and the second lateral portion is rolled around the visualization device and partially overlaps the first lateral portion.

Embodiment 27. A treatment device, comprising:
an elongate body;
a balloon coupled to the elongate body, the balloon comprising an electrode array and at least one pleat configured to facilitate flattening the balloon for placement into a delivery configuration;
a sheath configured to at least partially receive a visualization device and the balloon; and
wherein in the delivery configuration the balloon is at least partially positioned circumferentially around the visualization device within the sheath.

Embodiment 28. The treatment device of any of the preceding embodiments, wherein a distal portion of the balloon comprises the at least one pleat.

Embodiment 29. The treatment device of any of the preceding embodiments, wherein a distal portion of the balloon comprises the at least one pleat.

Embodiment 30. The treatment device of any of the preceding embodiments, wherein a lateral portion of the balloon comprises the at least one pleat.

Embodiment 31. The treatment device of any of the preceding embodiments, wherein the pleat is configured to stretch the target tissue.

Embodiment 32. The treatment device of any of the preceding embodiments, wherein the pleat is configured to transition the balloon between a folded configuration having a first diameter and an unfolded configuration having a second diameter greater than the first diameter.

Embodiment 33. The treatment device of any of the preceding embodiments, wherein the balloon comprises a wall thickness of between about 0.02 mm and about 0.5 mm.

Embodiment 34. The treatment device of any of the preceding embodiments, wherein the balloon comprises a seam formed via a thermal seal.

Embodiment 35. The treatment device of any of the preceding embodiments, wherein the balloon comprises a length of between about 10 mm and about 300 mm.

Embodiment 36. The device of any of the preceding embodiments, wherein the electrode array is configured to generate a therapeutic electric field at a first tissue depth of about 1 mm and a non-therapeutic electric field at a second tissue depth of at least about 1.5 mm.

Embodiment 37. The device of any of the preceding embodiments, wherein the electrode array defines one or more openings through the electrode array.

Embodiment 38. The device of any of the preceding embodiments, wherein one or more of the elongate body and the visualization device is configured to suction tissue through the one or more openings at a pressure between about 10 mmHg and about 200 mmHg.

Embodiment 39. The device of any of the preceding embodiments, wherein the electrode array is configured to generate a therapeutic electric field that treats a predetermined set of cell types and not muscularis tissue.

Embodiment 40. The device of any of the preceding embodiments, wherein the electrode array is configured to generate a therapeutic electric field that treats cells but leaves intact tissue scaffolding.

Embodiment 41. The device of any of the preceding embodiments, wherein the electrode array comprises a plurality of elongate electrodes comprising a ratio of a center-to-center distance between proximate electrodes to a width of the electrodes between about 2.3:1 and about 3.3:1.

Embodiment 42. The device of any of the preceding embodiments, wherein the plurality of elongate electrodes comprise a first electrode and a second electrode, wherein the second electrode is parallel to or interdigitated with the first electrode.

Embodiment 43. The device of any of the preceding embodiments, wherein the center-to-center distance between proximate electrodes and the width of the plurality of elongate electrodes is substantially equal.

Embodiment 44. The device of any of the preceding embodiments, wherein at least one of the electrodes comprises a semi-elliptical cross-sectional shape.

Embodiment 45. The device of any of the preceding embodiments, wherein a ratio of a height of an electrode to a width of an electrode is between about 1:4 and about 1:8.

Embodiment 46. The device of any of the preceding embodiments, wherein the proximate electrodes are spaced apart by a weighted average distance of between about 0.3 mm and about 6 mm.

Embodiment 47. The device of any of the preceding embodiments, wherein electrodes of the electrode array are spaced apart between about 0.5 mm and about 2 mm.

Embodiment 48. A system comprising the device of any of the preceding embodiments, further comprising a signal generator coupled to the electrode array, the signal generator configured to generate a pulse waveform comprising a frequency between about 250 kHz and about 950 kHz, a pulse width between about 0.5 μs and about 4 μs, a voltage applied by the electrode array of between about 100 V and about 2 kV, and a current density between about 0.6 A and about 100 A from the electrode array per square centimeter of tissue.

Embodiment 49. A method of treating a target tissue, comprising:
advancing a tissue treatment system and a visualization device to the target tissue of a patient, the tissue treatment system comprising,
   a tissue treatment device comprising an elongate body and an expandable member including an electrode array, and
   a sheath,
wherein in a delivery configuration, the expandable member is disposed in the sheath circumferentially about the visualization device in an unexpanded configuration;
advancing the expandable member distal to the sheath;
transitioning the expandable member into an expanded configuration;
treating the target tissue using the tissue treatment device; and
retracting the expandable member to reposition the system to the delivery configuration.

Embodiment 50. The method of any of the preceding embodiments further comprising visualizing the expandable member using the visualization device positioned within the sheath.

Embodiment 51. The method of any of the preceding embodiments further comprising repositioning the treatment device and treating a second target tissue.

Embodiment 52. The method of any of the preceding embodiments, wherein the treatment device is repositioned proximally or distally to treat the second target tissue before retracting the expandable member to reposition the system to the delivery configuration.

Embodiment 53. The method of any of the preceding embodiments further comprising overlapping the first lateral portion at least partially with the second lateral portion in the delivery configuration.

Embodiment 54. The method of any of the preceding embodiments, wherein overlapping the first lateral portion at least partially with the second lateral portion in the delivery configuration comprises positioning the first portion circumferentially about the visualization device in a first direction and positioning the second portion circumferentially about the visualization device in a second, opposite direction.

Embodiment 55. The method of any of the preceding embodiments, wherein advancing the expandable member distal to the sheath comprises translating the elongate member relative to the sheath.

Embodiment 56. The method of any of the preceding embodiments, wherein transitioning the expanded member to an expanded configuration comprises inflating the expandable member via an inflation lumen of the elongate body.

Embodiment 57. The method of any of the preceding embodiments further comprising applying suction to a portion of the target tissue through the expandable member.

Embodiment 58. The method of any of the preceding embodiments further comprising sizing the target tissue of the patient by advancing a sizing device.

Embodiment 59. The method of any of the preceding embodiments further comprising sizing the target tissue of the patient based on a pressure measurement.

Embodiment 60. The method of any of the preceding embodiments, wherein treating the target tissue treats a metabolic disorder comprising one or more of obesity, Non-alcoholic fatty liver disease (NAFLD), Nonalcoholic steatohepatitis (NASH), proinflammatory processes, immunological processes, Alzheimer's disease, neurological disorders, Type I diabetes, and Type II diabetes.

Embodiment 61. The method of any of the preceding embodiments, wherein the target tissue comprises one or more of a duodenum, a pylorus, an esophagus, a stomach, a small intestine, a large intestine, a vasculature, a thoracic cavity, an abdomino-pelvic cavity, a pelvic cavity, a vertebral cavity, and a cranial cavity.

Embodiment 62. A system for treating tissue, comprising:
a visualization device comprising a distal portion and a handle;
an inflation lumen coupled to the distal handle of the visualization device;
a balloon coupled to a distal end of the inflation lumen, the balloon comprising an electrode array, a distal pleat, and a proximal tapered portion; and
a sheath comprising a lumen coupled the proximal portion of the visualization device, the sheath at least partially receiving the inflation lumen and the balloon within the lumen,
wherein in a delivery configuration the balloon is positioned within the sheath lumen and a first portion of the balloon is rolled around the visualization device a first direction and a second portion of the balloon is rolled around the visualization device a second, opposite direction.

Embodiment 63. A treatment device comprising:
an overtube comprising a lumen and a window positioned along a sidewall of the overtube, the lumen and the window each configured to receive a visualization device therethrough; and
an expandable member coupled to the overtube, the expandable member configured to treat tissue, wherein the expandable member comprises one or more of a thermoplastic urethane, thermoplastic elastomer, polyethylene terephthalate, polyimide, nylon, and biaxially-oriented polyethylene terephthalate.

Embodiment 64. The treatment device of embodiment 63, wherein the visualization device is configured to advance through the window to visualize the expandable member.

Embodiment 65. A treatment device, comprising:
an elongate body; and
a balloon coupled to the elongate body, the balloon comprising at least one pleat configured to facilitate flattening of the expandable member for placement into a delivery configuration, wherein a width of the pleat in the delivery configuration is about 0.1 mm to about half of a difference between a width of the expandable member in the delivery configuration and a diameter of the elongate body, wherein the balloon is configured to treat tissue.

Embodiment 66. The treatment device of embodiment 65, wherein the at least one pleat comprises a first pleat on a first side of the balloon and a second pleat on a second side of the expandable member opposite the first side of the balloon.

Embodiment 67. The treatment device of embodiment 65, wherein the treatment device comprises an electrode array configured to treat the tissue, and wherein the electrode array is spaced apart from one or more of a proximal end and a distal end of the expandable member by at least about 0.25 inches.

Embodiment 68. The treatment device of embodiment 65, further comprising an electrode array coupled to the balloon.

Embodiment 69. The treatment device of embodiment 65, wherein the at least one pleat is configured to transition the balloon between a folded configuration having a first diameter and an unfolded configuration having a second diameter greater than the first diameter.

Embodiment 70. The treatment device of embodiment 65, wherein a lateral portion of the balloon comprises the at least one pleat.

Embodiment 71. The treatment device of embodiment 65, wherein the balloon comprises one or more of a thermoplastic urethane, thermoplastic elastomer, polyethylene terephthalate, polyimide, nylon, and biaxially-oriented polyethylene terephthalate.

Embodiment 72. A method of manufacturing a tissue treatment device, comprising:
disposing an electrode array on a surface of an expandable member, the electrode array comprising a substrate comprising one or more apertures along a perimeter of the substrate;
disposing a bonding layer over the electrode array; and
bonding the electrode array between the expandable member and the bonding layer using the apertures of the substrate.

Embodiment 73. The method of embodiment 72, further comprising attaching a first longitudinal edge of the expandable member to a second longitudinal edge of the expandable member to define a lumen of the expandable member.

Embodiment 74. The method of embodiment 73, wherein the first end of the expandable member comprises a first portion of a pleat, and the second end of the expandable member comprises a second portion of the pleat.

Embodiment 75. The method of embodiment 72, further comprising forming at least one pleat in the expandable member.

Embodiment 76. The method of embodiment 72, further comprising coupling an elongate body to an inner surface of the expandable member.

Embodiment 77. The method of embodiment 72, wherein the bonding layer covers a perimeter of the electrode array.

Embodiment 78. The method of embodiment 72, wherein the electrode array is bonded to the expandable member and the bonding layer using one or more of heat, pressure, an adhesive, and a chemical.

Embodiment 79. The method of embodiment 72, wherein the expandable member comprises one or more of a thermoplastic urethane, thermoplastic elastomer, polyethylene terephthalate, polyimide, nylon, and biaxially-oriented polyethylene terephthalate.

Embodiment 80. A treatment device, comprising:
an elongate body;
an expandable member coupled to the elongate body;
an electrode array coupled to the expandable member, the electrode array comprising a substrate defining a plurality of apertures; and
a bonding layer bonded to the electrode array and the expandable member using the apertures of the substrate.

Embodiment 81. The treatment device of embodiment 80, wherein the bonding layer covers a perimeter of the electrode array.

Embodiment 82. The treatment device of embodiment 80, wherein the substrate is sandwiched between the bonding layer and the expandable member.

Embodiment 83. The treatment device of embodiment 80, wherein the elongate body is an overtube.

Embodiment 83. The treatment device of embodiment 80, wherein the expandable member comprises a balloon.

Embodiment 84. The treatment device of embodiment 80, wherein the bonding layer is bonded to the expandable member through the electrode array using one or more of heat, pressure, an adhesive, and a chemical.

Embodiment 85. The treatment device of embodiment 80, wherein the expandable member comprises one or more of a thermoplastic urethane, thermoplastic elastomer, polyethylene terephthalate, polyimide, nylon, and biaxially-oriented polyethylene terephthalate.

Embodiment 86. The treatment device of embodiment 80, wherein the electrode array comprises a flexible circuit substrate.

Embodiment 87. The treatment device of embodiment 86, wherein the flexible circuit substrate comprises one or more of the group consisting of: all-Polyimide laminate, Polyester (PET), Polyethylene Naphthalate (PEN), Polyamide, Liquid Crystal Polymer (LCP), and PTFE

It should be understood that the examples and illustrations in this disclosure serve exemplary purposes and departures and variations such as the number of electrodes and devices, and so on can be built and deployed according to the teachings herein without departing from the scope of this invention.

As used herein, the terms “about” and/or “approximately” when used in conjunction with numerical values and/or ranges generally refer to those numerical values and/or ranges near to a recited numerical value and/or range. In some instances, the terms “about” and “approximately” may mean within ±10% of the recited value. For example, in some instances, “about 100 [units]” may mean within ±10% of 100 (e.g., from 90 to 110). The terms “about” and “approximately” may be used interchangeably.

The specific examples and descriptions herein are exemplary in nature and variations may be developed by those skilled in the art based on the material taught herein without departing from the scope of the present invention, which is limited only by the attached claims.

1. A treatment device, comprising:
an overtube comprising a lumen, the lumen configured to receive a visualization device therethrough;
an expandable member coupled to the overtube, the expandable member configured to treat tissue, wherein the expandable member is configured to generate a therapeutic electric field.

2. The treatment device of claim 1, further comprising a window through the overtube and in communication with the lumen, wherein the window is adjacent and proximal to the expandable member.

3. The treatment device of claim 2, wherein the window comprises a width of at least an outer diameter of the visualization device and a length greater than the width.

4. The treatment device of claim 1, wherein a distal end of the overtube is coupled to an inner diameter of the expandable member.

5. The treatment device of claim 2, wherein the overtube comprises a support disposed opposite or adjacent to the window, the support configured to increase a stiffness of the overtube.

6. The treatment device of claim 5, wherein the support comprises one or more of coil reinforcement, wire reinforcement and braid reinforcement.

7. The treatment device of claim 1, wherein the overtube comprises a stiffness of about 0.1 times to about 10 times a stiffness of a visualization device having a diameter configured to be disposed within the tissue treatment device.

8. The treatment device of claim 1, wherein the overtube comprises one or more of an inflation lumen, a suction lumen, a pull wire, and a lead wire.

9. The treatment device of claim 2, wherein the window is configured to receive the visualization device therethrough.

10. The treatment device of claim 1, wherein the expandable member comprises a treatment member configured to treat the tissue.

11. The treatment device of claim 10, wherein the treatment member comprises one or more of an electrode, an electrode array, a piezoelectric transducer, a laser, a blade, and a thermal radio frequency generator.

12. The treatment device of claim 11, wherein the treatment member comprises an electrode array coupled to the expandable member via a thermal seal.

13. The treatment device of claim 1, wherein the expandable member comprises a balloon.

14. The treatment device of claim 13, wherein the balloon comprises at least one pleat configured to transition the balloon between a folded configuration having a first diameter and an unfolded configuration having a second diameter greater than the first diameter.

15. The treatment device of claim 1, wherein a lateral portion of the expandable member comprises at least one pleat.

16. The treatment device of claim 1, wherein the expandable member comprises one or more of a thermoplastic urethane, thermoplastic elastomer, polyethylene terephthalate, polyimide, nylon, and biaxially-oriented polyethylene terephthalate.

17. A method of treating a target tissue, comprising:
advancing a tissue treatment system and a visualization device to the target tissue of a patient, the tissue treatment system comprising a tissue treatment device comprising an overtube defining a lumen, and an expandable member coupled to the overtube;
visualizing the target tissue with the visualization device before treating the target tissue;
transitioning the expandable member into an expanded configuration; and
treating the target tissue using the expandable member.

18. The method of claim 17 further comprising advancing the visualization device through the lumen of the overtube and distal to the expandable member before advancing the tissue treatment system and the visualization device to the target tissue.

19. The method of claim 18 further comprising a window defined though the overtube and advancing the tissue treatment device relative to the visualization device such that the window is distal to the visualization device after advancing the tissue treatment system and the visualization device to the target tissue.

20. The method of claim 19, further comprising visualizing one or more of the expandable member and the target tissue after the visualization device is advanced through the window of the overtube.

21. The method of claim 20, wherein visualizing the target tissue comprises identifying an ampulla of the duodenum.

22. The method of claim 17, further comprising applying suction to a portion of the target tissue through one or more of the visualization device and the lumen of the overtube.

23. The method of claim 17, further comprising re-treating the target tissue one or more times using the expandable member.

24. The method of claim 23, further comprising visualizing the treated target tissue before re-treating the target tissue.

25. The method of claim 17 further comprising repositioning the tissue treatment device and treating another target tissue.

26. The method of claim 18, wherein the window is in communication with the lumen of the overtube.

27. The method of claim 17, wherein the treatment device is repositioned proximally or distally of the target tissue to treat a second target tissue after transitioning the expandable member into the delivery configuration.

28. The method of claim 27, wherein the treatment device is rotatably repositioned to treat the second target tissue after transitioning the expandable member into the delivery configuration.

29. The method of claim 17, wherein transitioning the expandable member to the expanded configuration comprises inflating the expandable member via an inflation lumen of the overtube.

30. The method of claim 17, wherein the tissue treatment system and the visualization device are advanced to the target tissue of a patient in a delivery configuration where the expandable member is in an unexpanded configuration.

31. The method of claim 30, further comprising transitioning the expandable member back into the delivery configuration.

32. The method of claim 17, wherein treating the target tissue treats a metabolic disorder comprising one or more of obesity, Non-alcoholic fatty liver disease (NAFLD), Nonalcoholic steatohepatitis (NA SH), proinflammatory processes, immunological processes, Alzheimer's disease, neurological disorders, Type I diabetes, and Type II diabetes.

33. The method of claim 17, wherein treating the target tissue treats Barrett's esophagus.

34. The method of claim 17, wherein the target tissue comprises one or more of a duodenum, a pylorus, an esophagus, a stomach, a small intestine, a large intestine, a vasculature, a thoracic cavity, an abdomino-pelvic cavity, a pelvic cavity, a vertebral cavity, and a cranial cavity.

35. A treatment device, comprising:
an elongate body; and
an expandable member coupled to the elongate body, the expandable member comprising an electrode array and a plurality of pleats configured to facilitate flattening of the expandable member in an unexpanded configuration, wherein when transitioning the expandable member to an expanded configuration, a portion comprising the electrode array expands before the plurality of pleats unfold.

36. The treatment device of claim 35, wherein each pleat of the plurality of pleats comprises a first tapered portion coupled to a second tapered portion.

37. The treatment device of claim 36, wherein each of the plurality of pleats folds inwards in the unexpanded configuration.

38. The treatment device of claim 35, wherein the portion of the expandable member comprising the electrode array comprises a first rigidity and one or more other portions of the expandable member comprise a second rigidity different than the first rigidity.

39. The treatment device of claim 35, wherein a width of each pleat of the plurality of pleats is up to half of a difference between a width of the electrode array and a diameter of the elongate body.

40. The treatment device of claim 35, wherein the electrode array comprises a plurality of electrodes, and wherein the device is configured to maintain a predetermined spacing between the electrodes of the plurality of electrodes when the expandable member is expanded from an unexpanded configuration to a diameter of between about 15 mm and about 45 mm.

41. The treatment device of claim 40, wherein the expandable member comprises a balloon.

42. The treatment device of claim 41, wherein the plurality of pleats is configured to transition the balloon between a folded configuration having a first diameter and an unfolded configuration having a second diameter greater than the first diameter.

43. The treatment device of claim 35, wherein a lateral portion of the expandable member comprises at least one pleat of the plurality of pleats.

44. The treatment device of claim 35, wherein the expandable member comprises one or more of a thermoplastic urethane, thermoplastic elastomer, polyethylene terephthalate, polyimide, nylon, and biaxially-oriented polyethylene terephthalate.

45. The treatment device of claim 35, wherein one or more of the pleats comprise at least a portion of the electrode array.

46. The treatment device of claim 45, further comprising one or more impedance measurements configured to determine whether the one or more pleats comprising at least a portion of the electrode array is in a folded or unfolded configuration.

47. The treatment device of claim 35, further comprising one or more impedance measurements configured to determine whether sufficient contact between the electrode array and tissue is present.
